SEQUENCE LISTING (45 sequences)

SEQ ID NO: 1 - DNA, 23 bp, synthetic construct
  misc_feature 1..23: note = synthetic primer
  source 1..23: mol_type = other DNA
agaagctcattgttgggatcctg

SEQ ID NO: 2 - DNA, 25 bp, synthetic construct
  misc_feature 1..25: note = Synthetic primer
  source 1..25: mol_type = other DNA
acaatgagaactctattccctgaaa

SEQ ID NO: 3 - DNA, 20 bp, synthetic construct
  misc_feature 1..20: note = synthetic primer
  source 1..20: mol_type = other DNA
agctaagagacatccctccg

SEQ ID NO: 4 - DNA, 21 bp, synthetic construct
  misc_feature 1..21: note = synthetic primer
  source 1..21: mol_type = other DNA
ctctgtcactctacctgggtg

SEQ ID NO: 5 - RNA, 21 nt, synthetic construct
  misc_feature 1..21: note = syntheric gRNA
  source 1..21: mol_type = other RNA
aacaactatcgttaaccacag

SEQ ID NO: 6 - RNA, 20 nt, synthetic construct
  misc_feature 1..20: note = synthetic gRNA
  source 1..20: mol_type = other RNA
aaccagtattaggagactct

SEQ ID NO: 7 - RNA, 20 nt, synthetic construct
  misc_feature 1..20: note = synthetic gRNA
  source 1..20: mol_type = other RNA
gacttccgccgagagaacga

SEQ ID NO: 8 - DNA, 798 bp, synthetic construct
  misc_feature 1..798: note = Synthetic single-chain variable fragment
  source 1..798: mol_type = other DNA
atggccctgcccgtgaccgctctcctgctgcctctggccctgctcctccatgctgccagacccgacatccagatgacacagacaaccagcagcctgtccgcttccctcggagacagggtgacaatttcctgcagggccagccaggacatcagcaagtacctgaactggtaccagcagaaacccgacggcaccgtcaagctcctgatctaccacaccagcagactgcacagcggagtgccttccaggttcagcggcagcggctccggcaccgattactccctgaccattagcaacttagaacaggaggacattgccacctacttttgtcagcagggcaacaccctcccctacacctttggaggcggaaccaagttagaaatcaccggcggcggcggcagcggaggaggaggcagcggaggcggaggctccgaggtgaaactgcaggagagcggccccggactggtcgcccctagccaatccctctccgtcacctgcaccgtgagcggagtgagcctgcctgactacggagtgagctggatcagacagccccctaggaaaggactggaatggctgggcgtgatttggggcagcgagaccacctattacaacagcgccctgaagtccagactgacaatcatcaaggacaatagcaaaagccaagtgtttctgaagatgaacagcctgcagaccgatgacaccgccatctattattgcgccaagcactactactacggaggaagctacgctatggattattggggccaaggcacaagcgtgaccgtcagcagcgcggccgcc

SEQ ID NO: 9 - DNA, 921 bp, synthetic construct
  misc_feature 1..921: note = Synthetic single-chain variable fragment
  source 1..921: mol_type = other DNA
atggccttaccagtgaccgccttgctcctgccgctggccttgctgctccacgccgccaggccgatggaaaaggatacactgttgttgtgggttctgctcctgtgggtgcccggcagcaccggagatattgtgctgacgcagtctcctgcatcactcgccgtgtctctgggccagcgcgctaccatcagctgcagagcctctgaaagtgttgacaattatggaatttctttcatgaattggttccagcagaagcctggccagcccccgaaactcctcatatatgccgcgtctaatcagggctctggggtccctgctagattttctggcagcggctccggcaccgacttcagtctgaatatacatcccatggaagaagacgataccgccatgtacttttgccaacaatctaaggaggtgccttggacgttcggcggcggtacgaagctggaaattaagggcggcgggggaagcggcggggggggatcaggcgggggtggctccggaggcggtggaagtatgggctggagttggatcttccttttccttctttctggtaccgcgggagtgcactctgaggtgcagctccagcagtccggccccgagctcgtcaagcctggggccagtgtcaagatttcctgtaaggcatctggatatacctttacagattacaatatgcattgggtgaaacagtcacatggaaagtcactcgagtggatcggatacatttacccttacaatggaggaaccggatataatcagaagtttaagagcaaggccacactcacggtggacaattcttcatctacagcctacatggatgttcggtctctgacttccgaggatagtgcggtgtattactgcgccaggggacgccccgctatggattactgggggcagggaacctctgtaacagttagctca

SEQ ID NO: 10 - DNA, 1044 bp, synthetic construct
  misc_feature 1..1044: note = Synthetic single-chain variable fragment
  source 1..1044: mol_type = other DNA
atggccttaccagtgaccgccttgctcctgccgctggccttgctgctccacgccgccaggccggacttcgtgatgactcagtctcctagctccctgaccgtgacagccggcgagaaggtgaccatgtcctgcaaatctagtcagagtctgctgaactcaggcaatcagaagaactatctgacatggtacctgcagaagccagggcagccccctaaactgctgatctattgggccagcaccagggaatccggcgtgcccgacagattcaccggctccgggtctggaacagattttactctgaccatttcaagcgtgcaggccgaggacctggctgtgtactattgtcagaatgattacagctatccctacacatttggcgggggaactaagctggaaatcaaaggtggtggtggttctggtggtggtggttccggcggcggcggctccggtggtggtggatccgaggtgcagctgcagcagagtggacccgaactggtgaaacctggcgcctccgtgaaaatgtcttgcaaggctagtgggtacaccttcacagactactatatgaaatgggtgaagcagtcacacgggaagagcctggagtggatcggagatatcattccctctaacggcgccactttctacaatcagaagtttaaaggcaaggctactctgaccgtggaccggagctcctctaccgcctatatgcacctgaacagtctgacatcagaagatagcgctgtgtactattgtacacggtcccatctgctgagagcctcttggtttgcttattggggccaggggacactggtgactgtgagctccgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtg

SEQ ID NO: 11 - DNA, 252 bp, Homo sapiens
  source 1..252: mol_type = unassigned DNA
tccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 12 - DNA, 72 bp, Homo sapiens
  source 1..72: mol_type = unassigned DNA
ccattttttttctgctgcttcatcgctgtagccatgggaatccgtttcattattatggtaacaatatggagt

SEQ ID NO: 13 - DNA, 252 bp, Homo sapiens
  source 1..252: mol_type = unassigned DNA
cacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 14 - DNA, 73 bp, Homo sapiens
  source 1..73: mol_type = unassigned DNA
gtctctttctgcttggtgatggtactcctttttgcagtggacacaggactatatttctctgtgaagacaaaca

SEQ ID NO: 15 - DNA, 252 bp, Homo sapiens
  source 1..252: mol_type = unassigned DNA
tgtagaatctaccgcccttctgacaactctgtctctaagtccgtcagattctatctggtggtatctccagcctctgcctccacacagacctcctggactccccgcgacctggtctcttcacagacccagacccagagctgtgtgcctcccactgcaggagccagacaagcccctgagtctccatctaccatccctgtcccttcacagccacagaactccacgctccgccctggccctgcagcccccattgcc

SEQ ID NO: 16 - DNA, 72 bp, Homo sapiens
  source 1..72: mol_type = unassigned DNA
ctggtgcctgtgttctgtggactcctcgtagccaagagcctggtgctgtcagccctgctcgtctggtggggg

SEQ ID NO: 17 - DNA, 252 bp, Homo sapiens
  source 1..252: mol_type = unassigned DNA
tccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggg

SEQ ID NO: 18 - DNA, 72 bp, Homo sapiens
  source 1..72: mol_type = unassigned DNA
gctggtacagtcctcctccttcgggctggattctatgctgtcagctttctctctgtggccgtgggcagcacc

SEQ ID NO: 19 - DNA, 252 bp, Homo sapiens
  source 1..252: mol_type = unassigned DNA
ttccccctgggccctgtgaccacagcccacagagggacataccgatgttttggctcctataacaaccatgcctggtctttccccagtgagccagtgaagctcctggtcacaggcgacattgagaacaccagccttgcacctgaagaccccacctttcctgcagacacttggggcacctaccttttaaccacagagacgggactccagaaagaccatgccctctgggatcacactgcccagaatctccttcgg

SEQ ID NO: 20 - DNA, 72 bp, Homo sapiens
  source 1..72: mol_type = unassigned DNA
atgggcctggcctttctagtcctggtggctctagtgtggttcctggttgaagactggctcagcaggaagagg

SEQ ID NO: 21 - DNA, 252 bp, Homo sapiens
  source 1..252: mol_type = unassigned DNA
agggaaggggaggcccatgaacgtaggctccctgcagtgcgcagcatcaacggaacattccaggccgactttcctctgggccctgccacccacggagggacctacagatgcttcggctctttccgtgacgctccctacgagtggtcaaactcgagtgatccactgcttgtttccgtcacaggaaacccttcaaatagttggccttcacccactgaaccaagctccaaaaccggtaaccccagacacctacat

SEQ ID NO: 22 - DNA, 69 bp, Homo sapiens
  source 1..69: mol_type = unassigned DNA
gttctgattgggacctcagtggtcaaaatccctttcaccatcctcctcttctttctccttcatcgctgg

SEQ ID NO: 23 - DNA, 210 bp, Homo sapiens
  source 1..210: mol_type = unassigned DNA
atgaataaacaaagaggaaccttctcagaagtgagtctggcccaggacccaaagcggcagcaaaggaaacctaaaggcaataaaagctccatttcaggaaccgaacaggaaatattccaagtagaattaaatcttcaaaatccttccctgaatcatcaagggattgataaaatatatgactgccaaggtttactgccacctccagagaag

SEQ ID NO: 24 - DNA, 78 bp, Homo sapiens
  source 1..78: mol_type = unassigned DNA
ctcactgccgaggtcctaggaatcatttgcattgtcctgatggccactgtgttaaaaacaatagttcttattcctttc

SEQ ID NO: 25 - DNA, 252 bp, Homo sapiens
  source 1..252: mol_type = unassigned DNA
gtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 26 - DNA, 72 bp, Homo sapiens
  source 1..72: mol_type = unassigned DNA
atctacatctgggcgcccttggccgggacttgtggggtccttctcctgtcactggttatcaccctttactgc

SEQ ID NO: 27 - DNA, 252 bp, Homo sapiens
  source 1..252: mol_type = unassigned DNA
gcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacacc

SEQ ID NO: 28 - DNA, 84 bp, Homo sapiens
  source 1..84: mol_type = unassigned DNA
attccgtggctcggccacctcctcgtgggcctcagcggggcttttggcttcatcatcttagtgtacttgctgatcaactgcagg

SEQ ID NO: 29 - DNA, 126 bp, Homo sapiens
  source 1..126: mol_type = unassigned DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 30 - DNA, 182 bp, Homo sapiens
  source 1..182: mol_type = unassigned DNA
aaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaag

SEQ ID NO: 31 - DNA, 512 bp, Homo sapiens
  source 1..512: mol_type = unassigned DNA
tttctcagggagtattgctaaaatgccaaaaaggaagttgttcaaatgccactcagtatgaggacactggagatctaaaagtgaataatggcacaagaagaaatataagtaataaggacctttgtgcttcgagatctgcagaccagacagtactatgccaatcagaatggctcaaataccaagggaagtgttattggttctctaatgagatgaaaagctggagtgacagttatgtgtattgtttggaaagaaaatctcatctactaatcatacatgaccaacttgaaatggcttttatacagaaaaacctaagacaattaaactacgtatggattgggcttaactttacctccttgaaaatgacatggacttgggtggatggttctccaatagattcaaagatattcttcataaagggaccagctaaagaaaacagctgtgctgccattaaggaaagcaaaattttctctgaaacctgcagcagtgttttcaaatggatttgtcagtattag

SEQ ID NO: 32 - DNA, 251 bp, Homo sapiens
  source 1..251: mol_type = unassigned DNA
agaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtg

SEQ ID NO: 33 - DNA, 242 bp, Homo sapiens
  source 1..242: mol_type = unassigned DNA
attccctatctttgtctactcagcgaacacagggccccgcagagtccgcaaggaacctagagtatgtttcagtgtctccaacgaacaacactgtgtatgcttcagtcactcattcaaacagggaaacagaaatctggacacctagagaaaatgatactatcacaatttactccacaattaatcattccaaagagagtaaacccactttttccagggcaactgcccttgacaatgtcgtgtaa

SEQ ID NO: 34 - DNA, 167 bp, Homo sapiens
  source 1..167: mol_type = unassigned DNA
agtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaag

SEQ ID NO: 35 - DNA, 351 bp, Homo sapiens
  source 1..351: mol_type = unassigned DNA
aaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 36 - DNA, 210 bp, Homo sapiens
  source 1..210: mol_type = unassigned DNA
atccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctga

SEQ ID NO: 37 - DNA, 144 bp, Homo sapiens
  source 1..144: mol_type = unassigned DNA
aagcttttaatgataattcatgacagaagggagtttgctaaatttgaaaaggagaaaatgaatgccaaatgggacacgggtgaaaatcctatttataagagtgccgtaacaactgtggtcaatccgaagtatgagggaaaatga

SEQ ID NO: 38 - DNA, 141 bp, Homo sapiens
  source 1..141: mol_type = unassigned DNA
aaggctctgatccacctgagcgacctccgggagtacaggcgctttgagaaggagaagctcaagtcccagtggaacaatgataatccccttttcaagagcgccaccacgacggtcatgaaccccaagtttgctgagagttag

SEQ ID NO: 39 - DNA, 144 bp, Homo sapiens
  source 1..144: mol_type = unassigned DNA
aaactcctcatcaccatccacgaccgaaaagaattcgctaaatttgaggaagaacgcgccagagcaaaatgggacacagccaacaacccactgtataaagaggccacgtctaccttcaccaatatcacgtaccggggcacttaa

SEQ ID NO: 40 - DNA, 861 bp, Homo sapiens
  source 1..861: mol_type = unassigned DNA
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 41 - DNA, 573 bp, Homo sapiens
  source 1..573: mol_type = unassigned DNA
tataaagttgacttggttctgttctataggcgcatagcggaaagagacgagacactaacagatggtaaaacatatgatgcctttgtgtcttacctgaaagagtgtcatcctgagaataaagaagagtatacttttgctgtggagacgttacccagggtcctggagaaacagtttgggtataagttatgcatatttgaaagagatgtggtgcctggcggagctgttgtcgaggagatccattcactgatagagaaaagccggaggctaatcatcgttctcagccagagttacctgactaacggagccaggcgtgagctcgagagtggactccacgaagcactggtagagaggaagattaagatcatcttaattgagtttactccagccagcaacatcacctttctccccccgtcgctgaaactcctgaagtcctacagagttctaaaatggagggctgacagtccctccatgaactcaaggttctggaagaatcttgtttacctgatgcccgcaaaagccgtcaagccatggagagaggagtcggaggcgcggtctgttctctcagcaccttga

SEQ ID NO: 42 - DNA, 279 bp, Homo sapiens
  source 1..279: mol_type = unassigned DNA
aacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtga

SEQ ID NO: 43 - DNA, 660 bp, Homo sapiens
  source 1..660: mol_type = unassigned DNA
cattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 44 - DNA, 858 bp, Homo sapiens
  source 1..858: mol_type = unassigned DNA
agcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 45 - DNA, 1542 bp, Homo sapiens
  source 1..1542: mol_type = unassigned DNA
cccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctga

(19) United States
(12) Patent Application Publication
(10) Pub. No.: US 2024/0000843 A1
(43) Pub. Date: Jan. 4, 2024

(54) COMPOSITIONS AND METHODS FOR INHIBITING OR SCREENING FOR CD8 AND METHODS AND ASSAYS FOR DETECTING CD8 IN CELLS

(71) Applicant: Washington University, St. Louis, MO (US)

(72) Inventors: Todd Fehniger, St. Louis, MO (US); Melissa Berrien-Elliott, St. Louis, MO (US)

(21) Appl. No.: 18/323,898

(22) Filed: May 25, 2023

Related U.S. Application Data
(63) Continuation of application No. 17/154,889, filed on Jan. 21, 2021, now abandoned.
(60) Provisional application No. 62/963,971, filed on Jan. 21, 2020.

(51) Int. Cl. A61K 35/17; A61P 35/00; A61K 38/19; C07K 16/28; C07K 16/30; C12N 5/0783; C12N 15/113; G01N 33/50

(52) U.S. Cl. CPC A61K 35/17; A61P 35/00; A61K 38/19; C07K 16/2815; C07K 16/30; C12N 5/0646; C12N 15/1138; G01N 33/5005; C07K 2319/02; C07K 2319/03; C12N 2310/11; C12N 2310/14

(57) ABSTRACT

Among the various aspects of the present disclosure are provisions for methods of, and compositions for, increasing NK cell anti-tumor response, screening donors, and predicting response to NK cell therapy.

### CROSS-REFERENCE TO RELATED APPLICATIONS

This application is a continuation of U.S. application Ser. No. 17/154,889 filed 21 Jan. 2021, which claims priority from U.S. Provisional Application Ser. No. 62/963,971 filed on 21 Jan. 2020, which is incorporated herein by reference in its entirety.

### STATEMENT REGARDING FEDERALLY SPONSORED RESEARCH OR DEVELOPMENT

This invention was made with government support under CA205239 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention. The subject matter of the Sequence Listing is incorporated herein by reference in its entirety.

The present disclosure generally relates to, inter alia, natural killer (NK) cells including cytokine-induced memory like (CIML) NK cells, methods of making and using them e.g. in the treatment of cancer, increasing anti-tumor properties of NK cells.

Among the various aspects of the present disclosure is the provision of compositions and methods of increasing NK cell anti-tumor response, screening donors, and predicting response to NK cell therapy.

An aspect of the present disclosure provides for a method of increasing NK cell anti-tumor response in a subject in need thereof comprising: increasing CD8 loss-of-function or inhibiting, reducing, removing, or blocking CD8 expression, activity, or signaling in NK cells or progenitors thereof (e.g., genetic modification to remove or reduce CD8 activity or expression such as knocking out CD8, introducing a loss-of-function variant; blockade with anti-CD8 antibodies); enriching CD8-negative NK cells or progenitors thereof (e.g., expansion with cytokines, such as IL-12/15/18); and/or screening donor natural killer (NK) cells, prior to transplant into a subject, for a favorable fraction (i.e., a reduced fraction) of NKG2A+CD8+NK cells or progenitors thereof (e.g., the donor with the smallest fraction of NKG2A+CD8+ cells would be favorable).

In some embodiments, the NK cells or progenitors thereof are treated with a CD8 inhibiting agent in an amount effective to enhance anti-tumor response in NK cells or progenitors thereof.

Another aspect of the present disclosure provides for screening donor natural killer (NK) cells, prior to transplant into a subject: (i) obtaining or having obtained a biological sample from a donor; (ii) detecting the amount of CD8+ and/or CD8-negative NK cells; and/or (iii) detecting the expression of NKG2A.

In some embodiments, if the CD8 expression and, optionally, NKG2A expression on the donor cells is lower than that of a control or a non-responder (e.g., a median NKG2A expression (arcsinh) less than 30, a median CD8 expression (arcsinh) less than 2.5), the donor is considered a good candidate for donation.

In some embodiments, if the percent (%) double CD8⁺NKG2A⁺ cells are below that of a control, a non-responder, or 20%, the donor is considered a good candidate for donation.

Another aspect of the present disclosure provides for a method of reducing CD8 expression in NK cells in a subject in need thereof, comprising:
(i) obtaining or having obtained donor NK cells or progenitors thereof; and/or (ii) administering a therapeutically effective amount of a CD8 inhibiting agent.

In some embodiments, the subject has cancer.

In some embodiments, the NK cell or progenitor thereof can be genetically modified to remove or reduce CD8 expression (e.g., knocking out, introducing a loss-of-function variant.

In some embodiments, the CD8 inhibiting agent is chosen from:
an anti-CD8 antibody or functional fragment or variant thereof,
a short interfering RNA (siRNA) targeting CD8,
an antisense oligonucleotide (ASO) targeting CD8,
an inhibitory protein that antagonizes CD8,
a protein expression blocker (PEBL) targeting CD8, and
a fusion protein which is a decoy receptor for CD8.

In some embodiments, the CD8 inhibiting agent is administered in an amount effective to enhance anti-tumor response in NK cells or progenitors thereof.

Another aspect of the present disclosure provides a method of predicting response to NK cell therapy comprising: (i) obtaining or having obtained donor NK cells or progenitors thereof from a donor; and/or (ii) detecting an amount of CD8 expression.

In some embodiments, the method further comprises: (iii) detecting an amount of NKG2A expression.

In some embodiments, detecting an amount of CD8 and, optionally, NKG2A comprises: (i) purifying donor NK cell products; and/or (ii) detecting CD8+ and optionally NKG2A+ cells by mass cytometry.

Another aspect of the present disclosure provides for a method of any one of the preceding claims wherein the NK cell is a memory-like (ML) NK cells.

In some embodiments, the subject has cancer.

Another aspect of the present disclosure provides for a method of enriching CD8-negative or CD8-depleted NK cells comprising treating the NK cells with a cytokine or cytokine cocktail (e.g., IL-12/15/18) in an amount effective to expand the NK cells into CD8-negative-enriched or CD8-depleted memory-like (ML) NK cells.

Another aspect of the present disclosure provides for a method of treating cancer comprising administering a therapeutically effective amount of CD8-depleted NK cells or CD8-depleted ML NK cells to a subject in need thereof, wherein the therapeutically effective amount of CD8-depleted NK cells is in an amount effective to enhance anti-tumor response in NK cells compared to NK cells not CD8-depleted.

In some embodiments, the CD8-depleted NK cells or CD8-depleted ML NK cells are selected from a first donor with the most naturally occurring CD-negative enriched NK cells or ML NK cells compared to a second donor.

In some embodiments, the CD8-depleted NK cells or CD8-depleted ML NK cells are generated by treating NK cells with a cytokine or cytokine cocktail, such as IL-12/15/18, in an amount sufficient to increase the proportion of CD8-negative cells compared to cells not treated with a cytokine or cytokine cocktail.

Also provided are the following embodiments.

Embodiment 1. A method of increasing natural killer (NK) cell anti-tumor response in a subject in need thereof comprising:
increasing CD8 loss-of-function;
inhibiting, reducing, removing, or blocking CD8 expression, activity, or signaling in NK cells or progenitors thereof;
enriching CD8-negative NK cells or progenitors thereof; and/or
screening donors, prior to transplantation into a subject, for a favorable fraction of NKG2A+CD8+NK cells or progenitors thereof.

Embodiment 2. The method of Embodiment 1, wherein the inhibiting, reducing, removing, or blocking of CD8 expression, activity, or signaling is accomplished by genetic modification to remove or reduce CD8 activity or expression.

Embodiment 3. The method of Embodiment 2, wherein the inhibiting, reducing, removing, or blocking of CD8 expression, activity, or signaling is accomplished by knocking out CD8.

Embodiment 4. The method of Embodiment 1, wherein the inhibiting, reducing, removing, or blocking of CD8 expression, activity, or signaling is accomplished by blockade with an anti-CD8 antibody or functional fragment or variant thereof.

Embodiment 5. The method of Embodiment 1, wherein the inhibiting, reducing, removing, or blocking of CD8 expression, activity, or signaling is accomplished by administering a short interfering RNA (siRNA) targeting CD8.

Embodiment 6. The method of Embodiment 1, wherein the inhibiting, reducing, removing, or blocking of CD8 expression, activity, or signaling is accomplished by administering an antisense oligonucleotides (ASOs) targeting CD8.

Embodiment 7. The method of Embodiment 1, wherein the inhibiting, reducing, removing, or blocking of CD8 expression, activity, or signaling is accomplished by administering a protein that antagonizes CD8.

Embodiment 8. The method of Embodiment 1, wherein the inhibiting, reducing, removing, or blocking of CD8 expression, activity, or signaling is accomplished by administering an inhibitory protein which antagonizes CD8.

Embodiment 9. The method of Embodiment 1, wherein the protein which antagonizes CD8 is chosen from β-2 microglobulin and LPA5.

Embodiment 10. The method of Embodiment 1, wherein the inhibiting, reducing, removing, or blocking of CD8 expression, activity, or signaling is accomplished by administering a protein expression blocker (PEBL).

Embodiment 11. The method of Embodiment 1, wherein the inhibiting, reducing, removing, or blocking of CD8 expression, activity, or signaling is accomplished by administering a fusion protein which is a decoy receptor for CD8.

Embodiment 12. The method of Embodiment 1, wherein the increase in NK cell anti-tumor response is accomplished by enriching CD8-negative NK cells or progenitors thereof.

Embodiment 13. The method of Embodiment 12, wherein the CD8-negative NK cells or progenitors thereof are expanded with expansion with cytokines.

Embodiment 14. The method of Embodiment 13, wherein the cytokines are IL-12, IL-15, and IL-18, or functional fragments or variants thereof.

Embodiment 15. The method of Embodiment 13, wherein the cytokines are fusion proteins comprising functional fragments of variants of IL-12, IL-15, and IL-18.

Embodiment 16. The method of Embodiment 1, wherein the increase in NK cell anti-tumor response is accomplished by screening donor NK cells, prior to transplantation of the NK cells into a subject, for a favorable fraction of NKG2A+CD8+NK cells or progenitors thereof.

Embodiment 17. The method of Embodiment 16, wherein the favorable fraction of NKG2A+CD8+NK cells is lower than average, lower than that of a control, or lower than that of a non-responder.

Embodiment 18. The method of Embodiment 16, wherein the median NKG2A expression (measured in arcsinh) is less than 30 and the median CD8 expression (measured in arcsinh) is less than 2.5.

Embodiment 19. The method of Embodiment 1, wherein the increase in NK cell anti-tumor response is accomplished by genetic modification to remove or reduce CD8 activity or expression.

Embodiment 20. The method of Embodiment 19, wherein the genetic modification to remove or reduce CD8 activity or expression is a CD8 is introduction of a CD8 loss-of-function variant.

Embodiment 21. The method of Embodiment 19, wherein the genetic modification to remove or reduce CD8 activity or expression is a CD8 is CD8 knockout.

Embodiment 22. The method of Embodiment 19, wherein the genetic modification to remove or reduce CD8 activity or expression is genome editing done using CRISPR-Cas nucleases, TALENs, ZFNs, prime editors, or base editors.

Embodiment 23. The method of Embodiment 1, wherein the NK cells or progenitors thereof are treated with a CD8 inhibiting agent in an amount effective to enhance anti-tumor response in NK cells or progenitors thereof.

Embodiment 24. A method of screening donor natural killer (NK) cells, prior to transplant into a subject, comprising, in a biological sample obtained from the donor:
detecting the amount of expression of CD8+ and/or CD8-negative NK cells; and,
optionally,
detecting the amount of expression of NKG2A.

Embodiment 25. The method of Embodiment 24, wherein, if the CD8 expression and, optionally, NKG2A expression on the donor cells is lower than average, lower than that of a control, or lower than that of a non-responder, the donor is considered a good candidate for donation.

Embodiment 26. The method of Embodiment 24, wherein, if the median NKG2A expression (measured in arcsinh) is less than 30 and/or the median CD8 expression (measured in arcsinh) is less than 2.5, the donor is considered a good candidate for donation.

Embodiment 27. The method of Embodiment 24, wherein the amount of expression of both (i) expression of CD8+ and/or CD8-negative NK cells and (ii) amount of expression of NKG2A are detected.

Embodiment 28. The method of Embodiment 27, wherein, if the percent (%) double CD8⁺NKG2A⁺ NK cells is below average, below that of a control, below that of a non-responder, or below 20%, the donor is considered a good candidate for donation.

Embodiment 29. A method of reducing CD8 expression, activity, or signaling in donor NK cells that will be or have been transplanted from a donor into a subject in need thereof, comprising administering a therapeutically effective amount of a CD8 inhibiting agent.

Embodiment 30. The method of Embodiment 29, wherein the CD8 inhibiting agent is chosen from
an anti-CD8 antibody or functional fragment or variant thereof,
a short interfering RNA (siRNA) targeting CD8,
an antisense oligonucleotide (ASO) targeting CD8,
an inhibitory protein that antagonizes CD8,
a protein expression blocker (PEBL) targeting CD8, and
a fusion protein which is a decoy receptor for CD8.

Embodiment 31. The method of Embodiment 29, wherein the CD8 inhibiting agent comprises one or more cytokines, or one or more functional fragments or variants thereof, capable of expanding NK cells into CD8-deficient ML NK cells.

Embodiment 32. The method of either of Embodiments 29, wherein the CD8 inhibiting agent is administered in an amount effective to enhance anti-tumor response in NK cells or progenitors thereof.

Embodiment 33. A method of predicting response to NK cell therapy in a subject comprising, in donor NK cells or progenitors thereof, detecting the amount of CD8 expression.

Embodiment 34. The method of Embodiment 33, further comprising (ii) detecting the amount of NKG2A expression.

Embodiment 35. The method of either of Embodiments 33 and 34, wherein detecting CD8+ and optionally NKG2A+ cells is done by mass cytometry.

Embodiment 36. The method of any of Embodiments 33-35, wherein the detecting in donor NK cells is done prior to transplant into a subject.

Embodiment 37. The method of any of Embodiments 33-35, wherein the detecting in donor NK cells is done after transplant into a subject.

Embodiment 38. The method of Embodiment 33 wherein CD8 and, optionally, NKG2A expression on the donor cells that is lower than average, lower than that of a control, or lower than that of a non-responder predicts a better clinical response to NK cell therapy.

Embodiment 38. The method of Embodiment 33 wherein CD8 and, optionally, NKG2A expression on the donor cells that is lower than average predicts a better clinical response to NK cell therapy.

Embodiment 40. The method of Embodiment 33, wherein median NKG2A expression (measured in arcsinh) of less than 30 and/or the median CD8 expression (measured in arcsinh) of less than 2.5 predicts a better clinical response to NK cell therapy.

Embodiment 41. The method of any of the preceding Embodiments wherein the NK cells are memory-like (ML) NK cells.

Embodiment 42. The method of Embodiment # wherein the ML-NK cells are cytokine-induced memory-like (CIML) NK cells.

Embodiment 43. The method of any of the preceding Embodiments wherein the subject has cancer.

Embodiment 44. A method comprising enriching NK cells for CD8-negative NK cells or depleting CD8+NK cells and treating the NK cells with one or more cytokines, or one or more functional fragments or variants thereof, in an amount effective to expand the NK cells into CD8-negative-enriched or CD8-depleted memory-like (ML) NK cells.

Embodiment 45. The method of Embodiment 44, wherein the cytokines comprise IL-12, IL-15, and IL-18, or functional fragments or variants thereof.

Embodiment 46. The method of Embodiment 45, wherein the cytokines are fusion proteins comprising functional fragments or variants of IL-12, IL-15, and IL-18.

Embodiment 47. A method of treating cancer comprising administering to a subject in need thereof CD8-depleted NK cells in an amount effective to enhance anti-tumor response in NK cells compared to NK cells not CD8-depleted.

Embodiment 48. The method of Embodiment 47, wherein the CD8-depleted NK cells are enriched from a first donor with the most naturally occurring CD8-negative NK cells compared to a second donor.

Embodiment 49. The method of Embodiment 47, wherein the CD8-depleted NK cells are obtained from a donor with higher than average levels of CD8-negative NK cells.

Embodiment 50. The method of Embodiment 47, wherein median CD8 expression on CD8-depleted NK cells is less than average.

Embodiment 51. The method of Embodiment 47, wherein median CD8 expression (measured in arcsinh) on CD8-depleted NK cells is less than 2.5.

Embodiment 52. The method of Embodiment 47, wherein the CD8-depleted NK cells are generated by treating NK cells with one or more cytokines, or one or more functional fragments or variants thereof, in an amount sufficient to increase the proportion of CD8-negative cells compared to cells not treated with the cytokine(s) or fragment(s) or variant(s) thereof.

Embodiment 53. The method of any of Embodiments 44-52, wherein the cancer is AML.

Embodiment 54. The method of any of Embodiments 44-53 wherein the NK cells are memory-like (ML) NK cells.

Embodiment 55. The method of Embodiment 54 wherein the ML-NK cells are cytokine-induced memory-like (CIML) NK cells.

Embodiment 56. A population of cytokine-induced memory-like natural killer cells (CIML-NKs) or progenitors thereof that has reduced CD8 expression, activity, or signaling.

Embodiment 57. The population of CIML NK cells of Embodiment 56, that has reduced NKG2A expression, activity, or signaling.

Embodiment 58. The population of cells of either of Embodiments 56 and 57, that has reduced CD8 expression.

Embodiment 59. The population of CIML NK cells of either of Embodiments 56 and 57, that has reduced CD8 expression and reduced NKG2A expression.

Embodiment 60. The population of CIML NK cells of any of Embodiments 56-59, wherein the expression, activity, or signaling is reduced in comparison to a population of primary NK cells.

Embodiment 61. The population of CIML NK cells of any of Embodiments 56-59, wherein the favorable fraction of NKG2A+CD8+NK cells is lower than average, lower than that of a control, or lower than that of a non-responder.

Embodiment 62. The population of CIML NK cells of Embodiment 61, wherein the median NKG2A expression (measured in arcsinh) is less than 30 and the median CD8 expression (measured in arcsinh) is less than 2.5.

Embodiment 63. The population of CIML NK cells of any of Embodiments 56-62, wherein cells are obtained from a donor with higher than average levels of CD8-negative NK cells.

Embodiment 64. The population of CIML-NK cells of any of Embodiments 56-63, wherein median CD8 expression on CD8-depleted NK cells is less than average.

Embodiment 65. The population of CIML NK cells of any of Embodiments 56-64, which have been produced by:

treating the NK cells with one or more cytokines, or one or more functional fragments or variants thereof, in an amount effective to produce a memory-like phenotype; and one or more of:

enrichment of NK cells from donors which have a favorable fraction of CD8+NKG2A+ NK cells or progenitors thereof;
enriching CD8-negative NK cells or progenitors thereof;
genetic modification to remove or reduce CD8 activity or expression;
administering an inhibitory protein that antagonizes CD8,
administering an antisense oligonucleotides (ASOs) targeting CD8,
administering a short interfering RNA (siRNA) targeting CD8,
blockade with an anti-CD8 antibody or functional fragment or variant thereof;
administering a fusion protein which is a decoy receptor for CD8, and/or
administering a protein expression blocker (PEBL).

Embodiment 66. The population of CIML NK cells of Embodiment 56-64, which have been produced by treating the NK cells with one or more cytokines, or one or more functional fragments or variants thereof, in an amount effective to expand the NK cells into CD8-negative-enriched or CD8-depleted memory-like (ML) NK cells.

Embodiment 67. The method of Embodiment 66, wherein the cytokines comprise IL-12, IL-15, and IL-18, or functional fragments or variants thereof.

Embodiment 68. The method of Embodiment 67, wherein the cytokines are fusion proteins comprising functional fragments or variants of IL-12, IL-15, and IL-18.

Embodiment 69. A chimeric-antigen-receptor-bearing cytokine-induced memory-like natural killer cell that has reduced CD8 expression, activity, or signaling (CD8-low CAR-CIML), wherein the CAR construct comprises:
an antigen-recognition domain which binds to a disease-associated antigen;
a transmembrane domain; and
at least one intracellular signaling domain.

Embodiment 70. The CD8-low CAR-CIML of Embodiment 69, wherein the disease-associated antigen is expressed on a malignant T cell.

Embodiment 71. The CD8-low CAR-CIML of Embodiment 70, wherein the antigen expressed on a malignant T cell is chosen from CD2, CD3, CD4, CD5, CD7, TCRA, and TCRβ.

Embodiment 72. The CD8-low CAR-CIML of Embodiment 69, wherein the disease-associated antigen is expressed on a malignant myeloid cell.

Embodiment 73. The CD8-low CAR-CIML of Embodiment 72, wherein the antigen expressed on a malignant myeloid cell is chosen from CD33, FLT3, CD123, and CLL-1.

Embodiment 74. The CD8-low CAR-CIML of Embodiment 69, wherein the disease-associated antigen is expressed on a malignant plasma cell.

Embodiment 75. The CD8-low CAR-CIML of Embodiment 74, wherein the antigen expressed on a malignant plasma cell is chosen from BCMA, CS1, CD38, CD79A, CD796, CD138, and CD19.

Embodiment 76. The CD8-low CAR-CIML of Embodiment 69, wherein the disease-associated antigen is expressed on a malignant B cell.

Embodiment 77. The CD8-low CAR-CIML of Embodiment 76, wherein the antigen expressed on a malignant B cell is chosen from CD19, CD20, CD21, CD22, CD23, CD24, CD25, CD27, CD38, and CD45.

Embodiment 78. The CD8-low CAR-CIML of Embodiment 77, wherein the antigen expressed on a malignant B cell is chosen from CD19 and CD20.

Embodiment 79. The CD8-low CAR-CIML of Embodiment 69, wherein the disease-associated antigen is chosen from CD19, CD33, CD123, CD20, BCMA, mesothelin, EGFR, CD3, CD4 BAFF-R, EGFR, HER2, gp120, and gp41.

Embodiment 80. The CD8-low CAR-CIML of any of Embodiments 69-79, wherein the transmembrane domain is chosen from NKG2D, FcγRIIIa, NKp44, NKp30, NKp46, actKIR, NKG2C, CD8a, and IL15Rb.

Embodiment 81. The CD8-low CAR-CIML of any of Embodiments 69-80, wherein the at least one intracellular signaling domain is chosen from 4-1BB, DNAM-1, NKp80, 2B4, NTBA, CRACC, CD2, CD27, one or more integrins, IL-15R, IL-18R, IL-12R, IL-21R, IRE1a, and combinations thereof.

Embodiment 82. The CD8-low CAR-CIML of any of Embodiments 69-81, wherein the at least one intracellular signaling domain is a transmembrane adapter.

Embodiment 83. The CD8-low CAR-CIML of any of Embodiments 69-82, further comprising a transmembrane adapter or hinge.

Embodiment 84. The CD8-low CAR-CIML of Embodiment 83, wherein the transmembrane adapter is chosen from FceR1γ, CD3, DAP12, DAP10, and combinations thereof.

Embodiment 85. The CD8-low CAR-CIML of Embodiment 81, wherein the one or more integrins are selected from the group consisting of ITGB1, ITGB2, ITGB3, and combinations thereof.

Embodiment 86. A pharmaceutical composition comprising a CD8-low CAR-CIML of any of Embodiments 69-85, and a pharmaceutically acceptable carrier.

Embodiment 87. The pharmaceutical composition of Embodiment 86, wherein the pharmaceutically acceptable carrier is suitable for IV delivery.

Embodiment 88. A pharmaceutical composition comprising an enriched population of natural killer (NK) cells or progenitors thereof that have reduced CD8 expression, activity, or signaling, and a pharmaceutically acceptable carrier.

Embodiment 89. The pharmaceutical composition of Embodiment 88, wherein the NK cells or progenitors thereof that have reduced CD8 expression, activity, or signaling are memory-like NK cells or progenitors thereof.

Embodiment 90. The pharmaceutical composition of Embodiment 89, wherein the memory-like natural killer NK cells or progenitors thereof are cytokine-induced memory-like natural killer cells (CIML-NKs) or progenitors thereof.

Embodiment 91. The pharmaceutical composition of any of Embodiments 88-90, wherein the population of cells has reduced NKG2A expression, activity, or signaling.

Embodiment 92. The pharmaceutical composition of any of Embodiments 88-91, wherein the pharmaceutically acceptable carrier is suitable for IV delivery.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A-1G. In vivo differentiated cytokine-induced ML NK cells are distinct from conventional and cytokine-activated NK cells. FIG. 1A, Clinical trial schema.

FIG. 1B-1G, Mass cytometry analysis of patient peripheral blood mononuclear cells (PBMC) 7 days post-NK cell infusion reveal unique multidimensional phenotype and predominance of donor ML NK cells. Baseline (blue), activated (red), and memory-like (green) NK-cell samples are indicated. FIG. 1B, Representative viSNE plot of PBMC from a normal donor and a patient, 7 days after NK-cell infusions (D7). FIowSOM identified populations are indicated. Treg, regulatory T cells. C-E, viSNE analyses were performed on CD45+CD34⁻CD14⁻CD19⁻CD3⁻CD56⁺HLA^{donor} NK cells after enrichment (CD56⁺ Donor), activation, and 7 days after NK-cell infusion (Donor ML NK). FIG. 1C, Distinct populations were identified on viSNE maps based on clustering with 25 markers; baseline (BL), activated (ACT), and ML. FIG. 1D, Representative viSNE plots of one donor. Numbers indicate the frequency of NK cells that fall within the gate. FIG. 1E, Summary data from D of BL, ACT, and ML NK cells, demonstrating consistent NK-cell changes across donors. FIG. 1F, Summary data from all patients showing frequency of FIowSOM gated populations from B; Dn., donor NK cells, as determined by HLA marker expression. FIG. 1G, Summary total cell populations from all patients. For summary data, lines represent the mean and error is represented as SEM.

FIG. 2A-2B. Mass cytometry reveals distinct phenotypic changes after activation and in vivo ML NK-cell differentiation. FIG. 2A, Representative viSNE maps showing BL, ACT, and ML NK populations, as defined in FIG. 1. Plot colors represent the median expression of the indicated marker. FIG. 2B, Summary from A. Data were tested for normal distribution using Shapiro-Wilk test. Normally distributed data were analyzed using RM-ANOVA with Holm-Sidak correction for multiple comparisons. Nonparametric data were analyzed using Friedman test with Dunn multiple test correction. Lines represent the mean, and error is represented as SEM.

FIG. 3A-3C. Donor in vivo differentiated ML NK cells traffic to the BM and are phenotypically similar to PB ML NK cells. FIG. 3A, Summary data from Citrus-gated lymphocyte populations in patients with BM assessed by mass cytometry at day 8 post-NK cell infusion. FIG. 3B, viSNE overlay of donor NK cells at BL (blue), donor NK cells in the PB (green), and donor ML NK cells in the BM, using the same clustering as in FIG. 1 and FIG. 2. FIG. 3C, Representative expression of indicated markers in BL NK cells and donor ML NK cells (indicated within the gate) from the PB and BM.

FIG. 4A-D. Donor ML NK cells exhibit polyfunctional responses to leukemia targets ex vivo. FIG. 4A, Functional assay schema. Briefly, patient PBMCs were collected 8 (n=4) or 14 (n=1) days after NK-cell infusion. Lymphocytes were isolated, stimulated with K562, and assessed for the indicated markers by mass cytometry. FIG. 4B, Frequency of donor NK cells producing the indicated protein after K562 stimulation. Data were analyzed using paired t test (parametric) or Wilcoxon (nonparametric). P values are indicated within the graph. FIG. 4C, Polyfunctional responses from patient's donor NK cells. Total indicates the frequency of cells producing at least one cytokine/chemokine. FIG. 4D, Functionality by licensing status is indicated for donor NK cells. Solid symbols indicate the KIR was licensed in the donor; open symbols indicate the KIR was unlicensed in the donor. All KIR are predicated to be licensed in the patient. The frequency of each single KIR⁺ subset is indicated for each KIR within each donor (below the graph). These data indicate that unlicensed ML NK cells are functional.

FIG. 5A-5O. Markedly increased NKG2A expression on ML NK cells within patient PBMC at day 7 is associated with treatment failure. FIG. 5A, Representative histogram of NKG2A expression on donor NK cells from a responder (R) or treatment-failure (TF) patient. FIG. 5B, Summary data of median NKG2A expression on R versus TF patients (n=5 and 3, respectively). Data were compared using Mann-Whitney test. FIG. 5C-5J, Control and memory-like NK cells were generated from normal donors in vitro and assessed. FIG. 5C, Representative histograms showing IFNγ expression by ML NK cells triggered with the indicated targets for 6 hours. Inset numbers depict percent IFNγ⁺. FIG. 5D, Summary showing the frequency of IFNγ⁺ cells from ML and control (C) NK cells triggered with the indicated targets. Mean and SEM are shown; data were compared with RM-ANOVA with Holm-Sidak correction for multiple comparisons. FIG. 5E, ML NK cell killing of K562 or HLA-E⁺ K562 tumor targets at the indicated effector-to-target (E:T) ratio. FIG. 5F, qRT-PCR showing KLRC1 increases in ML NK cells compared with control cells. FIG. 5G, Normal donor CD56^{dim} CD16⁺ cells were flow-sorted on the basis of NKG2A expression. Control or ML NK cells were assessed at day 7 for NKG2A expression (left) and Ki-67 (right). Summary data from 4-5 normal donors from 2-3 independent experiments. Mean and SEM are depicted and data were compared using two-way ANOVA. FIG. 5H, Flow cytometry data showing the percent GATA3⁺ NK cells over time in control or ML NK cells. FIG. 5I, Median EOMES expression data from control and ML NK cells. FIG. 5H-5I, Data are mean and SEM, compared using two-way ANOVA with Sidak correction. FIG. 5J, Representative plot showing coexpression of GATA3, EOMES, and colored by NKG2A median fluorescence intensity (MFI). Gates enclose the GATA3⁺EOMES⁺ NK cells. FIG. 5K-5O, Using CRISPR/Cas9, EOMES was deleted from NK cells prior to ML NK-cell differentiation. Control, WT ML, and EOMES ML NK cells were generated in vitro and assessed after 7 days. FIG. 5K, CRISPR/Cas9 Schema. FIG. 5L, Representative histograms showing expression of indicated markers. FIG. 5M, Summary from L. N and O, Representative flow plots showing IFNγ production in response to K562 targets. Numbers represent the frequency of cells within the indicated gate. FIG. 5O, Summary from N. FIG. 5L-5O, Mean and SEM are displayed, data were compared using RM-ANOVA. N=6 normal donors from four independent experiments, P values are indicated within the graphs.

FIG. 6A-6J. Preventing NKG2A: HLA-E interactions restore ML NK-cell responses to HLA-E⁺ tumor targets. ML or control NK cells were generated in vitro and stimulated with K562 or K562-HLA-E⁺. FIG. 6A, Representative histogram showing IFNγ expression in ML NK cells stimulated with K562-HLA-E⁺ targets in the presence of isotype of anti-NKG2A antibody. FIG. 6B, Summary data of ML NK cells stimulated with K562 or K562-HLA-E⁺ with isotype or anti-NKG2A antibody. FIG. 6C, K562-HLA-E⁺ target killing by ML NK cells incubated with isotype or anti-NKG2A antibody. Mean and SEM are displayed. FIG. 6D, Representative flow cytometry data assessing intracellular IFNγ production by NK cells stimulated with primary AML in the presence of isotype or anti-NKG2A antibody. FIG. 6E, Summary data from FIG. 6D. Data were compared using a two-way ANOVA. FIG. 6F-6J, NKG2A protein expression was reduced using CRISPR/Cas9 and gRNA to KLRC1, then ML or control NK cells were generated in vitro and stimulated. FIG. 6F, Experimental schema. FIG. 6G, Representative histogram of ML NK cells electroporated with NKG2A gRNA (bottom) compared with control-treated ML NK Cells. FIG. 6H, Summary data showing percent NKG2A+NK cells. I and J, Control or ΔNKG2A ML NK cells were incubated with K562-HLA-E⁺ and IFNγ measured. FIG. 6I, Representative histogram showing NKG2A+ML NK cell and ΔNKG2A ML NK cell IFNγ production. FIG. 6J, Summary data from FIG. 6I. Data are represented as mean and SEM. Data were compared using RM one-way ANOVA, and P values are indicated within the graphs.

FIG. 7A-7M. CD8 expression on ML NK cells from patient PBMC at day 7 is associated with treatment failure. FIG. 7A, Representative histogram of CD8 expression on donor NK cells from responder (R) and a treatment-failure (TF) patient. FIG. 7B, Summary data of median CD8 expression on R versus TF patients. Data are shown with Box-Whisker graph, with bars indicating min to max. Data were compared using Mann-Whitney test. C-E, Enriched donor NK cells (baseline) were assessed for CD8 and NKG2A. FIG. 7C, Experimental schema. FIG. 7D, Representative plot showing frequency of NKG2A+CD8⁺ NK cells present in the baseline donor NK cells; inset numbers depict frequency of cells within the gate. FIG. 7E, Summary from FIG. 7D. FIG. 7F, Freshly isolated NK cells were stimulated with IL12/15/18 and phosphorylation of the indicated markers assessed at the indicated time points for CD8⁺ and CD8⁻ NK cells. Phosphorylation was induced in all markers (P>0.05 for all conditions, one-sample t test, test value=1). No significant differences were observed between fold increases in phospho-markers between the CD8 subsets, as determined by two-way ANOVA. FIG. 7G-7H, CD8⁻ and CD8⁺ NK cells were enriched from normal donor PBMCs, labeled with CTV, and stimulated with IL12/15/18 for 16 hours. FIG. 7G, CTV dilution was measured by flow cytometry at day 7. Summary data were compared using a paired t test. FIG. 7H, Intracellular Ki-67 was also assessed. Summary data were compared using a paired t test. FIG. 7I, Median expression of Ki-67 on donor ML NK cells from TF and R patients at day 7 post-infusion. Data were compared using Mann-Whitney test. FIG. 7J-7M, CRISPR/Cas9 was utilized to delete CD8a from NK cells prior to 7-day ML NK differentiation. FIG. 7J, Experimental schema. FIG. 7K, Representative histogram of CD8a expression on WT or ΔCD8a ML NK cells. FIG. 7L, K562 target killing by WT or ΔCD8a ML NK cells. FIG. 7M, IFNγ, TNF, and CD107 on WT and ΔCD8a ML NK cells stimulated with IL12+IL15 or K562 tumor targets. Mean and SEM are depicted. Data from n=4 normal donors from two independent experiments were compared using RM-ANOVA (L) and paired t tests (M). P values are indicated within the graphs.

FIG. 8A-8D. Gating schema and lymphocyte subsets rel/ref AML patients treated with ML NK cell adoptive therapy. FIG. 8A, NK Gating schema. NK cells are identified as Live (Cisplatin⁻), CD34⁻CD45⁺CD14⁻CD19⁻CD3⁻CD56⁺. FIG. 8B, Representative example of how HLA staining was utilized to distinguish donor versus recipient NK cells, CD34⁻ CD45⁺CD14⁻CD19⁻ cells are shown. In this example, donor cells are HLA-negative, while recipient (CD3⁺) cells are HLA⁺. FIG. 8C, Heatmap displaying the expression of the indicated marker within the FIowSOM gated lymphocyte subsets. FIG. 8D, Frequency and total numbers of each lymphocyte population by response, 7 days post-infusion. Dn. Donor NK cells, based on HLA expression.

FIG. 9A-9C. Phenotypic differences in baseline, activated and ML NK cells. FIG. 9A, Representative histograms of the indicated markers, from FIG. 2. FIG. 9B, Summary data demonstrating median expression of the indicated markers. FIG. 9C, Summary data of percent positive of each indicated marker.

FIG. 10A-10E. Phenotypic differences in baseline, activated and ML NK cells for NKG2C+ donor. FIG. 10A, viSNE map of one patient (CIML020) with robust NKG2C⁺ Population. FIG. 10B, Median expression of the indicated markers in this patient, consistent with ML NK cell differentiation. FIG. 10C-10E, Recipient v Donor NK cells in the blood on D7, analyzed as in FIG. 2. FIG. 100, viSNE density map of CIML020 donor and recipient NK cells. FIG. 10D, Overlay viSNE plot of Donor (D) and Recipient (R) NK cells (left) and HLA-A2 staining (not included in the viSNE clustering). FIG. 10E, Median expression of the indicated markers on D v R NK cells.

FIG. 11A-11B. KIR Diversity prior to infusion and after in vivo ML differentiation. FIG. 11A, KIR diversity was examined on donor NK cells pre-infusion and from the PB on day 7. FIG. 11B, Frequency of the indicated KIR on donor NK cells, pre-infusion and at D7.

FIG. 12A-12C. HLA-E on tumor targets inhibits ML NK cell responses. FIG. 12A, Assay Schema. FIG. 12B-12C, Memory-like NK cells were generated in vitro, and stimulated with HLA-E⁺ or HLA-E⁻ primary AML. FIG. 12B, Representative histogram of two primary HLA-E⁺ (red, bottom) or HLA-E^{lo} (blue, top) AML samples. Black line indicates lymphocytes within the sample, color histogram represents AML blasts (CD45^{lo} CD34⁺). FIG. 12C, Summary data showing median IFN-γ expression on IFN-γ⁺ cells stimulated with HLA-E⁺ or HLA-E^{lo} primary AML. Mean and SEM are shown and compared using paired T-test from 5 normal donors, stimulated with 3 different primary AML over 3 independent experiments.

FIG. 13A-13D. HLA-E expression on tumor, myeloid and lymphoid populations within the bone marrow tumor microenvironment from patients prior to ML NK cell therapy. Patient BM aspirate obtained upon enrollment in the study, but prior to treatment was assessed using mass cytometry. FIowSOM was used to identify cell subsets within the BM and median HLA-E expression was compared between treatment failure patients and responders. FIG. 13A, Heatmap demonstrating the phenotype of each FIowSOM identified meta-cluster. FIG. 13B, Median HLA-E expression (left) and percent positive (right) on the tumor subset, as defined in A, for responders v TF. Data were compared using Mann-Whitney or unpaired t test. FIG. 13C, Median HLA-E on the indicated cell subset. FIG. 13D Percent HLA-E positive on the indicated cell subset. Data are represented as mean and SEM, and were compared using 2way ANOVA.

FIG. 14A-14F. Transcription factor expression in control and ML NK cells. Control and ML NK cells were generated in vitro and assessed. FIG. 14A, Eomes expression in CD56^{Bright} and CD56^{dim} subsets were assessed by intracellular flow cytometry at the indicated timepoints. FIG. 14B, GATA-3 expression in CD56^{Bright} and CD56^{dim} subsets were assessed by intracellular flow cytometry at the indicated timepoints. Data are mean and SEM, compared using 2way ANOVA with Sidaks correction, from 6 normal donors, 3 independent experiments. FIG. 14C, RNA from control and ML NK cells after 6 days in vitro was sequenced. GSEA comparing the top 12,000 expressed genes to a GATA-3 target gene list. FIG. 14D, Control and ML NK cells were generated in vitro and assessed after 7 days for the indicated transcription factors by flow cytometry FIG. 14E, Control and ML NK cells were generated in vitro and assessed after 7 days for the indicated transcription factors by qPCR. FIG. 14F, Control and ML NK cells were generated in vitro and assessed after 7 days for the indicated transcription factors by qPCR. Mean and SEM displayed. Data are from 5-6 normal donors in 2-3 independent experiments. Data were compared using Paired t tests.

FIG. 15. HLA-E on tumor targets inhibits ML NK cell responses via NKG2A. WT and ΔNKG2A ML NK cells were stimulated with HLAE⁺ K562 leukemia targets in the presence of IgG isotype or anti-NKG2C blocking antibody. Summary data showing percent IFN-γ by flow (n=4, 2 independent experiments). Mean and SEM are shown and compared using RM-ANOVA. P-values are indicated in the graphs.

FIG. 16A-16F CD8a expression on CD3⁻ CD56⁺ NK cells. Freshly isolated NK cells were assessed for CD8 α expression by flow cytometry. FIG. 16A, Representative flow plot depicting CD8α⁺ NK cells. The number represents the frequency of cells within the gate. FIG. 16B, Summary data showing CD8a expression on the indicated NK cell subsets. Data show mean and SEM. Data compared using paired t test. FIG. 16C-16G, Freshly isolated NK cells (baseline), control and ML NK cells were generated in vitro and assessed on Day 7. FIG. 16C, CD8α expression on flow sorted CD8α-negative cells at baseline, and day 7 (control and ML). FIG. 16D-16G Unsorted NK cells were assessed for the indicated markers and compared to T cells. FIG. 16D, Representative histogram comparing the indicated markers on Lin− (CD45⁺ CD3⁻ CD56⁻), CD8α⁺ NK cells (CD45⁺ CD3⁻ CD56⁺ CD8a⁺), and T cells (CD45⁺ CD3⁺ CD56⁺ CD8a⁺). FIG. 16E, Summary data from FIG. 16D. FIG. 16F Representative histogram comparing the indicate markers on T cells (black), γδ T cells (purple, CD45⁺ CD3⁺γδ TCR⁺), and iNKT cells (red, CD45⁺ CD3⁺ Vα24-Jα18 TCR⁺). FIG. 16G Summary data from FIG. 16F, Data are from 2 independent experiments, n=5 normal donors.

FIG. 17A-17B. NKG2A upregulation and proliferation during in vivo ML differentiation. FIG. 17A, Median Ki-67⁺ cells on NKG2A+ and NKG2A⁻ in vivo differentiated donor ML NK cells from TF v R patient peripheral blood on day 7 post-NK cell infusion. FIG. 17B, percent Ki-67⁺ cells on NKG2A+ and NKG2A⁻ in vivo differentiated donor ML NK cells from TF v R patient peripheral blood on day 7 post-NK cell infusion. Mean and SEM are depicted, data compared using 2-way ANOVA.

FIG. 18A-18B. CRISPR/Cas9 efficiency. FIG. 18A, DNA from ΔNKG2A and ΔEomes NK cells was isolated and NGS sequencing performed. Summary data from 4-5 donors from 2-3 independent experiments. FIG. 18B, Summary flow cytometry data from WT and ΔCD8a NK cells indicate efficient CRISPR/Cas9 gene editing. Data from 5 normal donors in 2 independent experiments were compared using Paired t test. Mean and SEM are depicted.

FIG. 19 shows increased NKG2A and CD8 expression on NK cells in patients 1 week post ML NK cell infusion are associated with treatment failure (TF). Patient blood NK cells were examined one week after infusion by mass cytometry. Gating on NK cells (CD3−CD56⁺ NKp46⁺) identified that increased NKG2A and CD8 median expression were significantly associated with treatment failure (n=3-6 per group). The NKG2A association was previously discovered by the inventors, but the CD8 observation is new. Data were compared using Mann Whitney Test with post hoc multiple comparison correction.

FIG. 20 shows increased NKG2A+CD8⁺ NK cells in the donor at baseline is associated with treatment failure. Purified donor NK cell products were examined by mass cytometry. Gating on NK cells (CD3⁻CD56⁺ NKp46⁺) from donors whose recipients resulted in treatment failure (TF, n=3) and response (n=6) identified that increased NKG2A⁺ CD8⁺ NK cells were significantly associated with treatment failure. Data were compared using Mann Whitney Test.

FIG. 21A-21B shows CD8-deletion results in increased tumor target killing. NK cells were purified from normal donor PBMC. Using CRISPR/Cas9, CD8 was knocked out (+CD8 gRNA) or control treated (−gRNA) then activated with IL-12/15/18 (CIML). After 5 days, K562 killing by NK cells was assessed. FIG. 21A, CD8 expression by NK cells at 5 days after cytokine activation. FIG. 21B, Killing assay, representative of 3 independent experiments.

FIG. 22A-22D shows CD8-negative cells expand more robustly to IL-12/15/18 than CD8⁺ NK cells. NK cells were purified from normal donor PBMC and CD8-selected using magnetic beads. The cells were carboxyfluorescein succinimidyl ester (CFSE)-labeled and stimulated with IL-12/15/18 for 18 hours. The cytokines were washed away and the cells were allowed to differentiate. After 5 days, CFSE dilution was measured by flow cytometry. FIG. 22A, Flow cytometric analysis showing that CD8-negative cells diluted CFSE more than CD8⁺ NK cells. FIG. 22B, Data showing that CD8-negative cells diluted CFSE more than CD8⁺ NK cells. FIG. 22C, CD8⁻ cells had higher levels of Ki-67, a marker associated with cell division. FIG. 22D, In vivo patient data support that Ki-67 is reduced in TF patients, compared to Responder.

FIG. 23. NSG mice were engrafted with 1e⁶ HLA-E⁺ K562-luciferase, then 5e⁶ IL-12/15/18 activated NK cells from healthy donors with hi (>50%) or lo (<10%) CD8+ NK cells were infused. NK cells were supported with 3 doses rhIL-15/week. Bioluminescent imaging was performed at D14. Data were compared using Mann-Whitney.

### DETAILED DESCRIPTION

The present disclosure is based, at least in part, on the discovery that CD8+NK cells were associated with worse outcomes in a clinical trial. As shown herein, CD8 was expressed on memory-like NK cells following differentiation, assays were developed to test for associations with NK cell responses in an early phase clinical trial, and experiments demonstrated that CD8 loss of function resulted in enhanced anti-tumor response.

It has been previously suggested that CD8+NK cells are highly functional and exhibit enhanced responses to viruses and tumors. In direct contrast to this, here it was observed that CD8+NK cells were associated with worse outcomes in a clinical trial. In addition, when CD8 was removed from or inhibited in memory-like NK cells, there was enhanced anti-tumor function. Based on these unexpected results, new compositions and methods to treat cancer and other diseases, predict clinical responses to NK cell therapy, and enhance NK cell responses to tumors and viruses have been developed, as described herein.

Additionally, donors can be screened using CD8 status as a tool to choose the most suitable donor.

The cells and methods disclosed herein can be useful in the treatment of cancer (e.g., hematological cancer, solid tumors). Furthermore, the treatment efficacy can be enhanced using chimeric antigen receptors (CARs).

Natural Killer (NK) Cell-Based Therapy

As described herein, one aspect of the present disclosure provides a method for generating more potent memory-like NK cells to treat a proliferative disease, disorder, or condition (e.g., cancer, leukemia (e.g., AML), lymphomas, and solid tumors). For example, NK cells can be cytokine-induced memory-like NK cells (referred to as CIML or memory-like NK cells) having depleted levels of CD8 expression or with reduced or eliminated CD8 expression.

An aspect of the present disclosure provides for the preparation and use of NK cell-based therapy (e.g., preparing or selecting natural killer cells for cancer immunotherapy or adoptive cellular therapy) by combining IL-12, 15, and 18 preactivation and selection for reduced CD8 expression.

Another aspect of the present disclosure provides a method to improve NK cell therapy (such as adoptive cellular immunotherapy), the method comprising activating or co-culturing NK cells with IL-12, IL-15, and IL-18, selecting for CD8 depletion, depleting a population of CD8 positive cells, or reducing CD8 expression, and administering the NK cells to a subject. NK cells can be cytokine activated (i.e., cytokine induced) in vivo or in vitro.

Another aspect of the present disclosure provides for a new process of preparing natural killer cells for cancer immunotherapy, combining IL-12, 15, and 18 activation with reduction or elimination of CD8-positive cells or CD8 expression.

Treatments of various malignancies using NK cell-based therapies are well known in the art (see e.g., (i) Bachanova et al., NK Cells in Therapy of Cancer, Critical Reviews™ in Oncogenesis, 19(1-2):133-141 (2014) (ii) Tian et al., NK cell-based immunotherapy for malignant diseases, Cellular & Molecular Immunology (2013) 10, 230-252). Except as otherwise noted herein, therefore, the process of the present disclosure can be carried out in accordance with such processes.

Methods of treatments using NK cell therapies for several different types of cancer in clinical trials are well known; see e.g. Tian et al. 2013. Except as otherwise noted herein, therefore, the process of the present disclosure can be carried out in accordance with such processes.

Different approaches to NK-based immunotherapy, such as tissue-specific NK cells, killer receptor-oriented NK cells and chemically treated NK cells are well known; see e.g. Tian et al. 2013. Except as otherwise noted herein, therefore, the process of the present disclosure can be carried out in accordance with such processes.

Techniques or strategies to monitor NK cell therapy by non-invasive imaging, predetermine the efficiency of NK cell therapy by in vivo experiments and evaluate NK cell therapy approaches are well known; see e.g., Tian et al. 2013. Except as otherwise noted herein, therefore, the process of the present disclosure can be carried out in accordance with such processes.

As described herein, cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting NK cells capable of fighting cancer cells can be used as described herein.

Allogeneic cell therapy or allogenic transplantation uses donor cells from a different subject than the recipient of the cells. A benefit of an allogenic strategy is that unmatched allogeneic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

Natural Killer (NK) Cells

Natural killer (NK) cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are a promising alternative to T cells for allogeneic cellular immunotherapy since they have been administered safely without major toxicity, do not cause graft versus host disease (GvHD), naturally recognize and eliminate malignant cells, and are amendable to cellular engineering.

Natural killer (NK) cells play critical roles in host immunity against cancer. In response, cancers develop mechanisms to escape NK cell attack or induce defective NK cells. NK cells may be primary NK cells, or may be derived from progenitor cells. NK cells can be derived from various sources, including peripheral or cord blood cells, stem cells, or induced pluripotent stem cells (iPSCs), and a variety of stimulators can be used for large-scale production in laboratories or good manufacturing practice (GMP) facilities, including soluble growth factors, immobilized molecules or antibodies, and other cellular activators.

Memory-Like NK Cells

As described above, Natural Killer (NK) cells are cytotoxic innate lymphoid cells serving at the front line against infection and cancer. In inflammatory microenvironments, multiple soluble and contact-dependent signals modulate NK cell responsiveness. In addition to their innate cytotoxic and immunostimulatory activity, it has been uncovered in recent years that NK cells constitute a heterogeneous and versatile cell subset. Persistent memory-like NK populations that mount a robust recall response have been reported during viral infection, contact hypersensitivity reactions, and after stimulation by pro-inflammatory cytokines or activating receptor pathways.

Here is described the generation, functionality, and clinical applicability of memory-like NK cells. As described herein, the memory-like NK cell process has been improved using selection methods or synthetic biology. Examples disclosed herein include selecting for CD8⁺ depleted NK cells (i.e., CD8− cells), cytokine activation of NK cells, and, optionally, the addition of a chimeric antibody receptor (CAR) specific for use in ML NK cells.

Memory-like NK cells are potent anti-leukemia effectors. A process was previously discovered to enhance NK cell anti-tumor responses, memory-like differentiation following combined cytokine receptor activation (cytokine-induced memory-like NK cells, CIML NK cells). This was advanced pre-clinically and then clinically in the setting of leukemia immunotherapy.

As another example, increased CD56, Ki-67, NKG2A, and increased activating receptors NKG2D, NKp30, and NKp44 were observed for in vivo differentiated ML NK cells (see e.g., Example 1). In addition, in vivo differentiation showed modest decreases in the median expression of CD16 and CD11 b. Increased frequency of TRAIL, CD69, CD62L, NKG2A, and NKp30-positive NK cells were observed in ML NK cells compared with both ACT and BL NK cells, whereas the frequencies of CD27⁺ and CD127⁺ NK cells were reduced. Finally, unlike in vitro differentiated ML NK cells, in vivo differentiated ML NK cells did not express CD25.

Cytokine-Induced Memory-Like Natural Killer Cells (CIML-NKs)

Recent studies have shown that NK cells may acquire a memory-like phenotype, for example by viral infection or by preactivation with combinations of cytokines such as interleukin-12 (IL-12), IL-15, and IL-18, exhibiting enhanced response upon restimulation with the cytokines or triggering via activating receptors. As such, a memory-like NK cell can be a cytokine-induced memory-like (CIML) NK cells, which may be produced by activation with cytokines such as IL-12, IL-15, and IL-18 and their related family members, or functional fragments or variants thereof, or fusion proteins comprising functional fragments or variants thereof.

CIML NK cells may be identified by their method of production. CIML cells can be produced by differentiated cytokine-activated (i.e., CIML) NK cells.

It was discovered that ML NK cells resulting from in vivo differentiation are clearly distinct from conventional and activated NK cells and have a unique, consistent, well-defined multidimensional signature. As such, cytokine-induced (also known as cytokine-activated) memory-like NK cells typically exhibit differential cell surface protein expression patterns when compared to traditional NK cells. Such expression patterns are known in the art and may comprise, for example, increased CD56, CD56 subset CD56^{dim}, CD56 subset CD56^{bright}, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21, 8(357):357). Memory-like (ML) and cytokine induced memory-like (CIML) NK cells may also be identified by observed in vitro and in vivo properties, such as enhanced effector functions such as cytotoxicity, improved persistence, increased IFN-γ production, and the like.

As described herein, NK cells can be activated using cytokines, such as IL-12/15/18. The NK cells can be incubated in the presence of the cytokines for an amount of time sufficient to form cytokine-induced memory-like (CIML) NK cells. For example, the amount of time sufficient to form cytokine-induced memory-like (ML) NK cells can be between about 8 and about 24 hours, about 12 hours, or about 16 hours. As another example, the amount of time sufficient to form cytokine-induced memory-like (ML) NK cells can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

CD8 Inhibition

CD8 (cluster of differentiation 8) is a transmembrane glycoprotein that serves as a co-receptor for the T cell receptor (TCR). Like the TCR, CD8 binds to a major histocompatibility complex (MHC) molecule, but is specific for the class I MHC protein. There are two isoforms of the protein, alpha and beta, each encoded by a different gene. In lymphocytes, such as NK cells, the CD8 is found in the CDαα homodimeric form. The CD8αα co-receptor interacts with the MHC-I constant alpha domains and it also binds HLA-G, but not HLA-E.

As described herein, CD8 expression has been implicated in weak NK cell anti-tumor response. As such, modulation of CD8 can be used for enhancing NK cell anti-tumor response. A CD8 inhibiting agent can inhibit CD8 activity (function), CD8 expression, or enhance CD8 loss-of-function. CD8 inhibition can comprise modulating the expression of CD8 on cells, modulating the quantity or number of cells that express CD8, or modulating the quality of the CD8 expressing cells.

As such, the present disclosure provides for methods of treating cancer based on the discovery that CD8⁺ and/or NKG2A⁺ NK cells are associated with weaker tumor response than a population of cells having a higher proportion of CD8-negative and/or NKG2A-negative NK cells. Accordingly, inhibiting or reducing CD8 expression, activity, or signaling is expected to improve cell therapies including adoptive cell transfer therapeutics (e.g., NK cell therapy, ML NK cell therapy, stem and progenitor cell therapy).

For example, a CD8 inhibiting agent can be used. A CD8 inhibiting agent can be any composition or method that can inhibit, block, or reduce CD8 expression, CD8 activity, or CD8 signaling on cells (e.g., anti-CD8 antibodies). For example, a CD8 inhibiting agent can be an inhibitor or an antagonist. As another example, the CD8 inhibiting can be the result of gene editing.

A CD8 inhibiting agent can be an agent that inhibits progenitor cell differentiation into CD8 expressing cells (e.g., cytokines, such as IL-12/15/18). For example, a CD8 inhibiting agent can be used to block CD8 expression during differentiation.

CD8 Signal Reduction, Elimination, or Inhibition by Small Molecule Inhibitors, RNAs, or ASOs

A CD8 inhibiting agent can be any agent that can inhibit CD8 expression or activity, downregulate CD8 expression, or knockdown CD8 expression.

A CD8 inhibiting agent can be used to reduce/eliminate CD8 signals. For example, a CD8 inhibiting agent can be a small molecule inhibitor of CD8.

As another example, a CD8 inhibiting agent can be a short hairpin RNA (shrank) targeting CD8. As another example, a CD8 inhibiting agent can be a short interfering RNA (siRNA) targeting CD8. As another example, a CD8 inhibiting agent can be a single guide RNA (sgRNA) or a short interfering RNA (siRNA) targeting CD8. As another example, CD8 RNA can be targeted with antisense oligonucleotides (ASOs). Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

Antibodies, Fusion Proteins, Small Molecules

As described herein, inhibitors of CD8 (e.g., antibodies, fusion proteins, small molecules) can reduce or prevent CD8 expression or activity.

A CD8 inhibiting agent can be an anti-CD8 antibody (e.g., a monoclonal antibody to CD8) or a functional fragment or variant thereof, such as scFvs. Any CD8 antibody can be used, for example, those known in the art and those that are commercially available. Furthermore, the anti-CD8 antibody can be a monoclonal murine antibody, a monoclonal humanized murine antibody, or a monoclonal human antibody (or a functional fragment or variant thereof).

As another example, the CD8 inhibiting agent can be a fusion protein. For example, the fusion protein can be a decoy receptor for CD8. Furthermore, the fusion protein can comprise a mouse or human Fc antibody domain fused to the ectodomain of CD8.

As another example, a CD8 inhibiting agent can be an inhibitory protein that antagonizes CD8. For example, the CD8 inhibiting agent can be a protein, which has been shown to antagonize CD8 (e.g., β-2 microglobulin, LPA5). LPA5 has been shown to be a potent and specific inhibitor of CD8 signaling. β-2 microglobulin and derivatives thereof have been shown to be antagonists of CD8.

Methods for preparing a CD8 inhibiting agent (e.g., an agent capable of inhibiting CD8 signaling) can comprise construction of a protein/Ab scaffold containing the natural CD8 receptor as a CD8 neutralizing agent; developing inhibitors of the CD8 receptor “down-stream”; or developing inhibitors of the CD8 production “up-stream”.

Genome Editing

Inhibiting CD8 can be performed by genetically modifying CD8 in a subject or genetically modifying a subject to reduce or prevent expression of the CD8 gene, such as through the use of CRISPR (e.g. CRISPR-Cas9, CRISPR-Cpf1), transcription activator-like effector nucleases (TALENs), zinc finger nucleases, (ZFNs), prime editors comprising Cas9 and reverse transcriptase, base editors comprising a CRISPR protein that does not cause a double-stranded break and a base editing enzyme (e.g., a deaminase), or analogous technologies, wherein, such modification reduces or prevents CD8 expression, signaling, or activity. Adequate blockage of CD8 by genome editing can result in increased anti-tumor activity of the NK cells.

As described herein, CD8 signals can be modulated (e.g., reduced, eliminated) using genome editing. Processes for genome editing are well known; see e.g., Aldi 2018 Nature Communications 9 (1911). Except as otherwise noted herein, therefore, the process of the present disclosure can be carried out in accordance with such processes.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for NK cell therapy to target cells by the removal of CD8 signals.

For example, the methods as described herein can comprise a method for altering a target polynucleotide sequence in a cell comprising contacting the polynucleotide sequence with a clustered regularly interspaced short palindromic repeats-associated (Cas) protein.

In a variation of the method above, a construct encoding one or more protein expression blocker (PEBL) may be transduced into the cell, either as the editing step or part of the editing step, or as part of CAR transduction. For example, a construct encoding an antibody-derived single-chain variable fragment specific for CD8 may be transduced, e.g. by a lentiviral vector. Once expressed, the PEBL colocalizes intracellularly with CD8, blocking surface CD8 expression. PEBLs may be produced for blockade of any of the targets of gene suppression disclosed herein.

Chimeric Antigen Receptor (CAR) Constructs

The present disclosure provides for incorporation of a process improvement that provides new ways to have ML NK cells respond to recognize many antigens, for example, on a variety of tumor types, beyond the established biology of NK cell activating and inhibitory receptors. Specifically, this disclosure provides for the genetic modification of ML NK cells capable of responding, via a synthetic artificial receptor, using a chimeric antigen receptor (CAR). CD19, CD33, and CD123-recognizing receptors can be used, directly, in ML NK cells against normally resistant B cell cancers with associated B cell antigens. This new platform can be used to perform many modifications of ML NK cells, to provide new recognition of antigens and tumors, provide new strategies to overcome inhibition, and enhance ML NK cell function, survival, and persistence. The design of these CARML NK cells provide novel possibilities and are based on ML NK cell biology.

The present disclosure provides for ML NK cells modified with CARs. It is believed that the present disclosure is the first to design these CAR constructs capable of being incorporated into NK cells, more specifically, ML NK cells.

CAR designs are generally tailored to each cell type. The present disclosure is drawn to ML NK cells, but could be useful in other immune cell types. As such, ML NK cells can be engineered to express chimeric antigen receptors (CARs).

CARs are designed in a modular fashion that comprise an extracellular target-binding domain, a hinge region, a transmembrane domain that anchors the CAR to the cell membrane, and one or more intracellular domains that transmit activation signals. Depending on the number of costimulatory domains, CARs can be classified into first (CD3z only), second (one costimulatory domain+CD3z), or third generation CARs (more than one costimulatory domain+CD3z). Introduction of CAR molecules into a ML NK cell successfully redirects the ML NK cell with additional antigen specificity and provides the necessary signals to drive full ML NK cell activation.

Because antigen recognition by CARML NK cells is based on the binding of the target-binding single-chain variable fragment (scFv) to intact surface antigens, targeting of tumor cells is not MHC restricted, co-receptor dependent, or dependent on processing and effective presentation of target epitopes.

Furthermore, the CAR construct moieties can be operably linked with a linker. A linker can be any nucleotide sequence capable of linking the moieties. For example, the linker can be any amino acid sequence suitable for this purpose (e.g., of a length of 9 amino acids).

The chimeric antigen receptor (CAR) can be transduced via a viral vector (e.g., lentivirus) into the cytokine-activated ML NK cells in the presence of IL-15 for an amount of time sufficient to virally transduce CAR into the cytokine-activated ML NK cells, resulting in CAR-transduced ML NK cells. For example, the amount of time sufficient to form CAR-transduced ML NK cells can be between about 12 hours and about 24 hours. As another example, the amount of time sufficient to virally transduce CAR into the ML NK cells (forming CAR-transduced ML NK cells) can be at least about 1 hour; about 2 hours; about 3 hours; about 4 hours; about 5 hours; about 6 hours; about 7 hours; about 8 hours; about 9 hours; about 10 hours; about 11 hours; about 12 hours; about 13 hours; about 14 hours; about 15 hours; about 16 hours; about 17 hours; about 18 hours; about 19 hours; about 20 hours; about 21 hours; about 22 hours; about 23 hours; about 24 hours; about 25 hours; about 26 hours; about 27 hours; about 28 hours; about 29 hours; about 30 hours; about 31 hours; about 32 hours; about 33 hours; about 34 hours; about 35 hours; about 36 hours; about 37 hours; about 38 hours; about 39 hours; about 40 hours; about 41 hours; about 42 hours; about 43 hours; about 44 hours; about 45 hours; about 46 hours; about 47 hours; or about 48 hours.

Next, the CAR-transduced ML NK cells can be incubated in the presence of IL-15 for an amount of time sufficient to express the vector and to form CAR-expressing ML NK (CARML NK cells). For example, the amount of time sufficient to form CARML NK cells can be between about 3 days and about 8 days. As an example, the amount of time sufficient to form CARML NK cells can be at least about 1 day; about 2 days; about 3 days; about 4 days; about 5 days; about 6 days; about 7 days; about 8 days; about 9 days; about 10 days; about 11 days; about 12 days; about 13 days; or about 14 days.

Targeting Antibody Fragment Against a Disease-Associated Antigen (e.g., Single-Chain Variable Fragments (scFvs))

Targeting antibody fragments against a disease-associated antigen can comprise Single-chain variable fragments (scFvs). scFvs can be any scFv capable of binding to a target antigen or target antigen epitope. For example, the scFvs can target an antigen associated with an infectious disease, a bacterial infection, a virus, or a cancer. scFvs can be against any antigen known in the art, such as those described in U.S. application Ser. No. 15/179,472, and is incorporated by reference in its entirety.

Targeting antibody fragments or scFvs can be against any tumor-associated antigen (TAA). A TAA can be any antigen known in the art to be associated with tumors.

scFvs, such as CD19, CD33, and CD123 CARs can be expressed on the ML NK cells. For example, CD19 can target cancer or deplete B cells for autoimmune diseases to remove autoantibodies. Other scFvs, such as scFvs that recognize: CD20, BCMA, Mesothelin, EGFR, CD3, CD4 BAFF-R, EGFR, HER2, HIV: gp120, or gp41 can also be incorporated into the CAR construct.

The antigen-binding capability of the CAR is defined by the extracellular scFv, not the targeted antigen. The format of a scFv is generally two variable domains linked by a flexible peptide sequence, either in the orientation VH-linker-VL or VL-linker-VH. The orientation of the variable domains within the scFv, depending on the structure of the scFv, may contribute to whether a CAR will be expressed on the ML NK cell surface or whether the CARML NK cells target the antigen and signal. In addition, the length and/or composition of the variable domain linker can contribute to the stability or affinity of the scFv.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

CAR scFv affinities, modified through mutagenesis of complementary-determining regions while holding the epitope constant, or through CAR development with scFvs derived from therapeutic antibodies against the same target, but not the same epitope, can change the strength of the ML NK cell signal and allow CARML NK cells to differentiate overexpressed antigens from normally expressed antigens. The scFv, a critical component of a CAR molecule, can be carefully designed and manipulated to influence specificity and differential targeting of tumors versus normal tissues. Given that these differences may only be measurable with CARML NK cells (as opposed to soluble antibodies), pre-clinical testing of normal tissues for expression of the target, and susceptibility to on-target toxicities, requires live-cell assays rather than immunohistochemistry on fixed tissues.

The scFvs described herein can be used for hematological malignancies such as AML, ALL, or Lymphoma, but can also be expanded for use in any malignancy, autoimmune, or infectious disease where a scFv can be generated against a target antigen or antigen epitope. For example, the constructs described herein can be used to treat or prevent autoimmunity associated with auto-antibodies (similar indications as rituximab for autoimmunity). As another example, the disclosed constructs can also be applied to virally infected cells, using scFv that can recognize viral antigens, for example, gp120 and gp41 on HIV-infected cells.

Transmembrane (TM) Domains and Adapters

The constructs described herein incorporate a transmembrane domain consisting of a hydrophobic a helix that spans the cell membrane. Although the main function of the transmembrane is to anchor the CAR in the ML NK cell membrane, previous evidence has also suggested that the transmembrane can be relevant for CAR cell function.

Others have previously looked at transmembrane (TM) domains for use in CAR, but do not work in known NK cells. The inventors discovered that, unexpectedly, the transmembrane domains that do not work in other NK cell CAR constructs work in ML NK cells, as described herein.

It was shown that CD8 TM moiety was applicable for ML NK cells, because ML NK cells are more mature and have different characteristics than other NK cells. This TM domain does not work in other NK cells (see e.g., Li et al. 2018 Cell Stem Cell 23181-192).

The TM domain can be any TM domain suitable for use in an NK cell or ML NK cell. For example, the TM domain can be a sequence associated with NKG2D, FcγRIIIa, NKp44, NKp30, NKp46, actKIR, NKG2C, or CD8a.

NK cells express a number of transmembrane (TM) adapters that signal activation, that are triggered via association with activating receptors. This provides an NK cell specific signal enhancement via engineering the TM domains from activating receptors, and thereby harness endogenous adapters. The TM adapter can be any endogenous TM adapter capable of signaling activation. For example, the TM adapter can be FceR1γ (ITAMx1), CD3ζ (ITAMx3), DAP12 (ITAMx1), or DAP10 (YxxM/YINM).

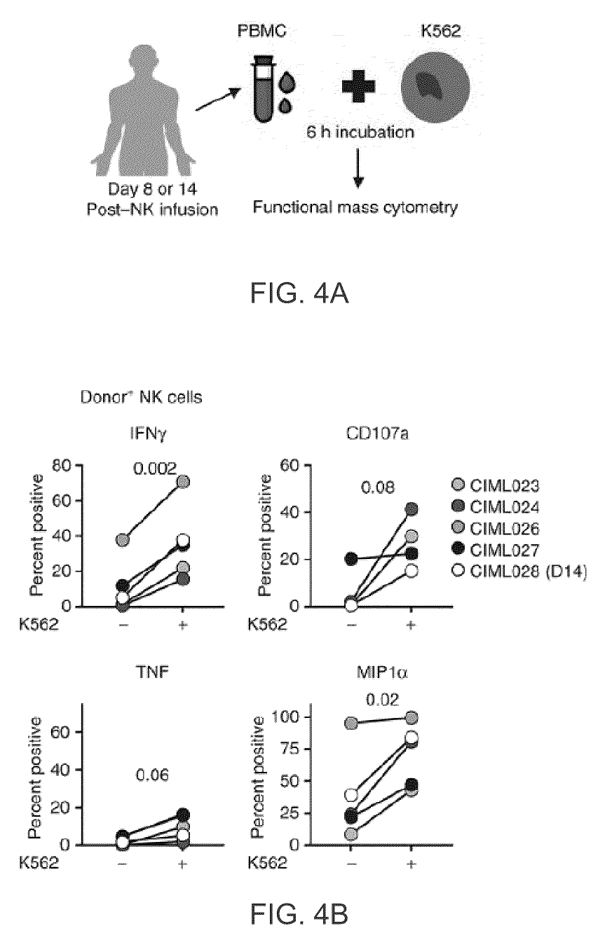

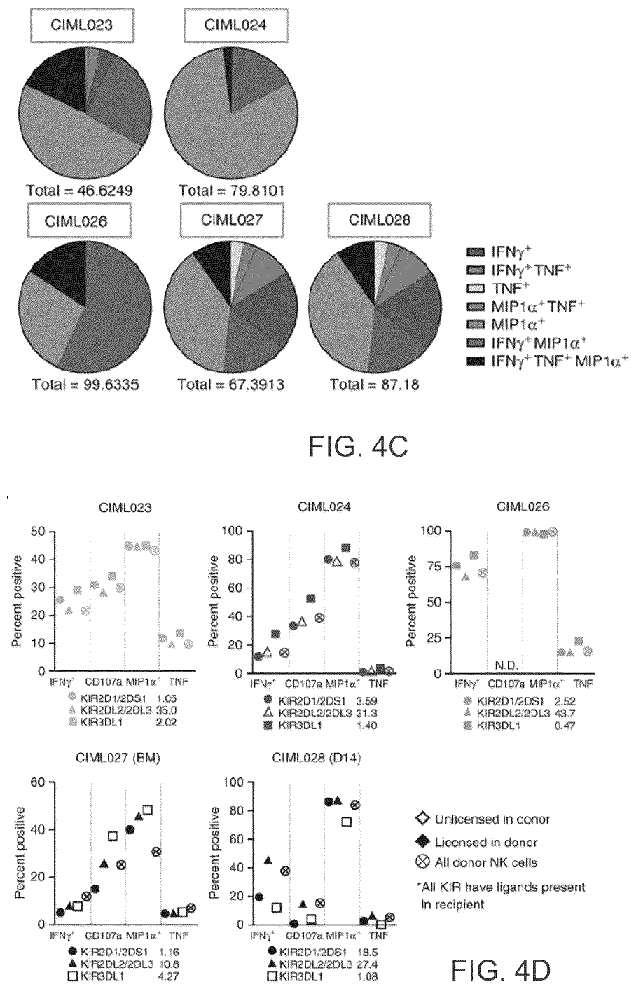

It was discovered that ML NK cells have increased NKG2D, NKp30, and NKp44 expression, providing a rationale for their use in ML NK cells. As shown in FIG. 4, NK cells express a number of transmembrane (TM) adapters that signal activation, that are triggered via association with activating receptors. This provides an NK cell specific signal enhancement via engineering the TM domains from activating receptors, and thereby harness endogenous adapters.

Hinge (Spacer)

The hinge, also referred to as a spacer, is in the extracellular structural region of the CAR that separates the binding units from the transmembrane domain. The hinge can be any moiety capable of ensuring proximity of the CARML NK cell to the target (e.g., NKG2-based hinge, TMα-based hinge, CD8-based hinge). With the exception of a few CARs based on the entire extracellular moiety of a receptor, such as NKG2D, the majority of CAR (such as CAR T) cells are designed with immunoglobulin (lg)-like domain hinges.

Hinges generally supply stability for efficient CAR expression and activity. The NKG2 hinge (also in combination with the transmembrane domain), described herein also ensures proper proximity to target.

The hinge also provides flexibility to access the targeted antigen. The optimal spacer length of a given CAR can depend on the position of the targeted epitope. Long spacers can provide extra flexibility to the CAR and allow for better access to membrane-proximal epitopes or complex glycosylated antigens. CARs bearing short hinges can be more effective at binding membrane-distal epitopes. The length of the spacer can be important to provide adequate intercellular distance for immunological synapse formation. As such, hinges may be optimized for individual epitopes accordingly. The hinge can be operably linked to the transmembrane domain.

Intracellular Signaling Domain (Costimulatory Domains)

The present disclosure provides for an intracellular signaling domain useful in ML NK cells. For example, others using NK cells were not able to use CD137 (4-1BB) in the NK cells, but surprisingly, these and others can work in the ML NK cells. The CAR construct can comprise one or more intracellular signaling domains.

NK cells can also utilize co-activating receptors to amplify activating signals. Signaling domains/motifs (SD) may be harnessed that are selectively expressed in ML NK cells (e.g., DNAM-1, CD137, CD2). Importantly, NK cells receive homeostasis, proliferation, and persistence signals from cytokine receptors, most notably the IL-2/15R. CARML NK cells may be further tailored to result in certain outcomes, including cytokine production, cytotoxicity, and long-term persistence.

In some embodiments, an intracellular signaling domain can be any co-activating receptor capable of functioning in an NK cell (e.g., a ML NK cell). For example, a co-activating receptor can be CD137/41BB (TRAF, NFkB), DNAM-1 (Y-motif), NKp80 (Y-motif), 2B4 (SLAMF) ITSM, CRACC (CS1/SLAMF7) ITSM, CD2 (Y-motifs, MAPK/Erk), CD27 (TRAF, NFkB), or integrins (e.g., multiple integrins).

In some embodiments, an intracellular signaling domain can be a cytokine receptor capable of functioning in an NK cell (e.g., a ML NK cell). For example, a cytokine receptor can be a cytokine receptor associated with persistence, survival, or metabolism, such as IL-2/15Rbyc Jak1/3, STAT3/5, PI3K/mTOR, MAPK/ERK. As another example, a cytokine receptor can be a cytokine receptor associated with activation, such as IL-18R::NFkB. As another example, a cytokine receptor can be a cytokine receptor associated with IFN-γ production, such as IL-12R STAT4. As another example, a cytokine receptor can be a cytokine receptor associated with cytotoxicity or persistence, such as IL-21R::Jak3/Tyk2, or STAT3.

As another example, an intracellular signaling domain can be a TM adapter, such as FceR1γ (ITAMx1), CD3 (ITAMx3), DAP12 (ITAMx1), or DAP10 (YxxM/YINM).

As another example, CAR intracellular signaling domains (also known as endodomains) can be derived from costimulatory molecules from the CD28 family (such as CD28 and ICOS) or the tumor necrosis factor receptor (TNFR) family of genes (such as 4-1BB, OX40, or CD27). The TNFR family members signal through recruitment of TRAF proteins and are associated with cellular activation, differentiation and survival.

As another example, CD28 and 4-1BB have been widely used costimulatory endodomains in CARs in T cells, but it is believed this is the first time these endodomains have been shown to work in NK cells. Clinical trials with CARs incorporating CD28 or 4-1 BB intracellular domains showed similar response rates in patients with hematologic malignancies for T cells, but has yet to be shown in NK cells until now.

The high effector function and self-limited expansion of CD28-based CARs may be ideal to transiently treat diseases with a rapid tumor elimination and short-term persistence of the CAR in ML NK cells (i.e., as a bridge therapy for allogeneic hematopoietic stem cell transplantation). Furthermore, 4-1BB-based CARs may be used to treat diseases in which complete response may require sustained NK cell persistence.

Other domains, such as incorporation of ICOS can be incorporated into a CARML NK cell. Recent data suggest that various lymphocyte subsets require distinct costimulation signals for optimal function and persistence. The ICOS intracellular domain can enhance the persistence of CARML NK cells and the 4-1BB intracellular domain can provide optimal persistence in CARML NK cells.

In some embodiments, the CARML NK cell can join the properties of different intracellular domains in one single ML NK cell by combining two or more intracellular domains in a CAR. For example, such combinations can include one intracellular domain from the CD28 family and one intracellular domain from the TNFR family, resulting in the simultaneous activation of different signaling pathways.

Each costimulatory domain can have unique properties. Differences in the affinity of the scFv, the intensity of antigen expression, the probability of off-tumor toxicity, or the disease to be treated may influence the selection of the intracellular domain.

Extracellular Signaling Domain

Optionally, an extracellular signaling domain can be incorporated into the CAR construct to propagate signaling. The extracellular signaling domain can be cloned into the hinge region, such as a CD8 hinge, but can be chosen based on the target.

Cell Screening

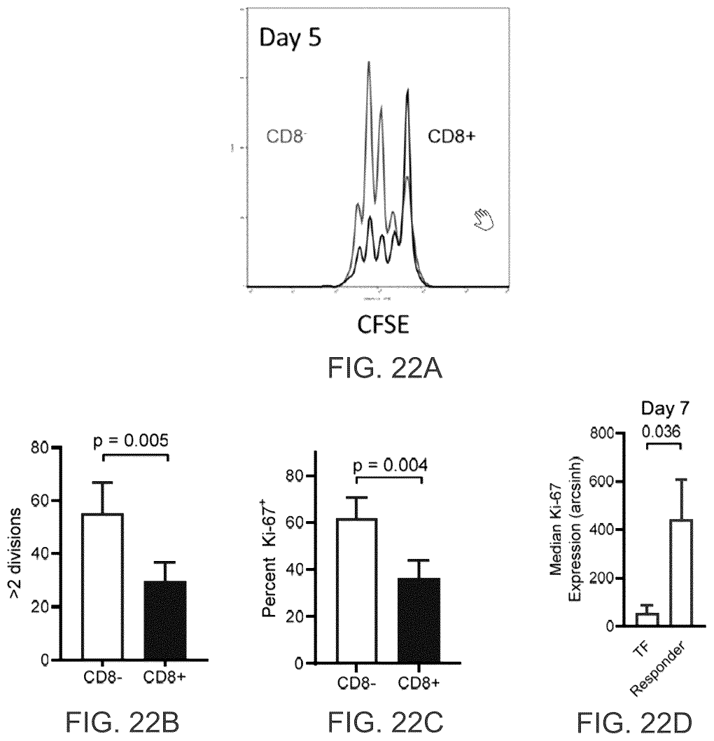

It was discovered here that CD8 is identified as a negative predictor of response to NK cell therapy. Furthermore, it was discovered here that CD8-negative cells expand more robustly to IL-12/15/18 than CD8⁺ NK cells. Results demonstrate that CD8⁺ NK cells have reduced proliferation in response to cytokine-induced memory-like differentiation than CD8-negative cells (see e.g., FIG. 22).

As such, also provided, are methods for screening donor NK cells for transplant into a subject having cancer comprising, in a biological sample obtained from the donor, (i) detecting the amount of CD8⁺ and/or CD8-negative NK cells; and/or (ii) detecting the expression of NKG2A. If the CD8 expression and, optionally, NKG2A expression on the donor cells is lower than that of a control or a non-responder, the donor is considered a good candidate for donation. In certain embodiments, median NKG2A expression (arcsinh) is less than 30 and/or median CD8 expression (arcsinh) is less than 2.5.

As such, donor cells can be chosen with a favorable fraction of CD8⁺ NKG2A+ cells (e.g., a low or reduced fraction). This can also allow for the prediction of treatment response based on baseline NK cell attributes from a donor.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

The term “expression vector,” or equivalently, “expression construct,” “recombinant DNA construct,” or sometimes “plasmid,” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art.

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation,” or “transduction,” e.g., when accomplished by means of a viral vector, refers to the transfer of a nucleic acid or fragment thereof into the genome of a host cell, resulting in genetically stable inheritance. Host cells containing the transformed nucleic acid fragments are referred to as “transgenic” cells, and organisms comprising transgenic cells are referred to as “transgenic organisms.” For example, transduction can be used to introduce foreign DNA into eukaryotic cells, like mammalian cell lines. This can be done with a viral vector such as a lentiviral vector or an Adeno Associated Viruses (AAV). Lentiviral vectors or AAVs can be used to create both transient cell lines, where a gene of interest is expressed but not integrated into the genome, or stable cell lines, where foreign DNA is incorporated into the cell's genome and is thus passed down through cell division.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

The term “transfection” refers to the transfer of a nucleic acid fragment into a host cell, typically by non-viral methods and typically resulting in transient expression of the encoded polypeptide.

“Wild-type” refers to a virus or organism, or a protein or gene therein, found in nature without any known mutation. The term wild type may include common natural polymorphisms.

Design, generation, and testing of the variant nucleotides, and their encoded polypeptides, having the above required percent identities and retaining a required activity of the expressed protein is within the skill of the art. For example, directed evolution and rapid isolation of mutants can be according to methods known in the art. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | |
|---|---|
| Non-polar (hydrophobic) | Amino Acid |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| GIn (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art. Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome. Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to overexpress. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art.

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated as pharmaceutical formulations/compositions by any conventional manner using one or more pharmaceutically acceptable carriers or excipients known in the art. Such formulations will contain a therapeutically effective amount of a biologically active agent described herein, which can be in purified form, together with a suitable amount of carrier so as to provide the form for proper administration to the subject.

The term “formulation,” or equivalently, “pharmaceutical composition,” refers to preparing an active pharmaceutical ingredient, e.g., a drug or biologic, in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art. Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. Agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. Where the product is, for example, a biologic or cell therapy, the mode of administration will likely be via injection or infusion. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of certain agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

NK cells are important for host protection against infectious pathogens and mediate anti-tumor immune responses. The agents and compositions described herein can be used to treat an infectious pathogen or a proliferative disease, disorder, or condition. NK cells protect against infectious pathogens and mediate anti-tumor immune responses by targeting cells associated with infectious pathogens and proliferative diseases, disorders, or conditions. Target cells can be any cell associated with an infectious pathogen, autoimmune disease, or a proliferative disease, disorder, or condition.

Provided herein is a process of treating a proliferative disease, disorder, or condition, infectious disease, or immune disorder in a subject in need of administration of a therapeutically effective amount of NK cell-based therapy (e.g., using genetically modified NK cells). The disclosed NK-cell based therapy can be used as a treatment for cancer (e.g., as an immunotherapy drug), for an autoimmune disease (e.g., treatment to deplete B cells), or for an infectious disease.

A proliferative disease, disorder, or condition can be a cancer. Accordingly, also provided is a process of treating, preventing, or reversing cancer in a subject in need of administration of a therapeutically effective amount of a CD8 inhibiting agent or CD8-deficient NK cells (NK cells deficient in CD8 activity, expression, or signaling), so as to increase the NK cell's anti-tumor activity.

In some embodiments, the compounds and pharmaceutical compositions of the present disclosure may be useful in the treatment or prevention of progression of cancer. The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), non-Hodgkin's lymphomas, B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL) Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

Solid tumors include melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

An infectious pathogen can be any infectious disease. For example, infections that can be treated with NK cells include, but are not limited to, viral infections (e.g., cytomegalovirus, Epstein Barr virus, herpes simplex virus, human immunodeficiency virus), intracellular pathogens (e.g., Listeria monocytogenes), bacterial infections, and fungal infections.

Methods described herein are generally performed on a subject in need thereof. A subject in need of the therapeutic methods described herein can be a subject having, diagnosed with, suspected of having, or at risk for developing a proliferative disease, disorder, or condition, such as cancer (e.g., hematological cancer and solid tumors). A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans, or other animals such as chickens. For example, the subject can be a human subject.

Generally, a safe and effective amount of an NK cell-based treatment, such as an NK cell therapy having reduced CD8 expression, CD8-deficient NK cells, or NK cells treated with a CD8 inhibiting agent is, for example, an amount that would cause the desired therapeutic effect in a subject while minimizing undesired side effects. In various embodiments, an effective amount of a CD8 inhibiting agent or CD8-deficient NK cells described herein can substantially inhibit CD8, slow the progress of cancer, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration. Where the product is, for example, a biologic or cell therapy, the mode of administration will likely be via injection or infusion.

When used in the treatments described herein, a therapeutically effective amount of a CD8 inhibiting agent or CD8-deficient NK cells can be employed in pure form or, where the compound is a chemical and such forms exist, in pharmaceutically acceptable salt form, and with or without a pharmaceutically acceptable excipient. For example, the compounds of the present disclosure can be administered, at a reasonable benefit/risk ratio applicable to any medical treatment, in a sufficient amount to inhibit, reduce, or remove CD8 expression, activity, or signaling.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts. For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a CD8 inhibiting agent or CD8-deficient NK cells can occur as a single event or over a time course of treatment. For example, a CD8 inhibiting agent or CD8-deficient NK cells can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for treating cancer.

A CD8 inhibiting agent or CD8-deficient NK cells can be administered simultaneously or sequentially with another agent, such as an anti-cancer agent or therapy. For example, a CD8 inhibiting agent or CD8-deficient NK cells can be administered simultaneously with another agent or treatment, such as chemotherapy, radiation, or immunotherapy. Simultaneous administration can occur through administration of separate compositions, each containing one or more of a CD8 inhibiting agent or CD8-deficient NK cells, an anti-cancer therapeutic, or another agent. Simultaneous administration can occur through administration of one composition containing two or more of a CD8 inhibiting agent or CD8-deficient NK cells, an anti-cancer therapeutic, or another agent. A CD8 inhibiting agent or CD8-deficient NK cells can be administered sequentially with an anti-cancer therapeutic, or another agent. For example, a CD8 inhibiting agent or CD8-deficient NK cells can be administered before or after administration of an anti-cancer therapeutic, or another agent.

Diseases, Disorders, or Conditions

NK cells are important for host protection against infectious pathogens and mediate anti-tumor immune responses. The agents and compositions described herein can be used to treat an infectious pathogen or a proliferative disease, disorder, or condition. NK cells protect against infectious pathogens and mediate anti-tumor immune responses by targeting cells associated with infectious pathogens and proliferative diseases, disorders, or conditions. Target cells can be any cell associated with an infectious pathogen or a proliferative disease, disorder, or condition.

Methods and compositions as described herein can be used for the prevention, treatment, or slowing the progression of a proliferative disease, disorder, or condition (e.g., cancer), autoimmune conditions associated with autoantibodies, immune disorder, or infectious diseases (e.g., bacterial, viral).

There are numerous cancers that are treated with NK cell-based therapies (e.g., (i) Bachanova et al., NK Cells in Therapy of Cancer, Critical Reviews™ in Oncogenesis, 19(1-2):133-141 (2014) (ii) Tian et al., NK cell-based immunotherapy for malignant diseases, Cellular & Molecular Immunology (2013) 10, 230-252). For example, cancers treated with NK-based cell therapies can be, but are not limited to, hematological cancer or a cancer with a solid tumor such as acute myeloid leukemia (AML); acute lymphoblastic leukemia; advanced non-small cell lung cancer; breast cancer; colon carcinoma; gastric carcinoma; Hodgkin's disease; lymphoma tumors; glioma, lung cancer; melanoma; melanoma; metastatic breast carcinoma; metastatic melanoma; metastatic renal cell carcinoma; multiple myeloma; neuroblastoma; non-Hodgkin's lymphoma; osteosarcoma; renal cell carcinoma; soft-tissue sarcoma; renal cell carcinoma; or ovarian cancer. For example, leukemias that can be treated with the NK cell-based therapy can be acute myeloid (or myelogenous) leukemia (AML); chronic myeloid (or myelogenous) leukemia (CML); acute lymphocytic (or lymphoblastic) leukemia (ALL); chronic lymphocytic leukemia (CLL); hairy cell; T-cell prolymphocytic; or juvenile myelomonocytic leukemia.

Additional cancers can be, for example, Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sezary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

As another example, the autoimmune condition or immune disorder can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosurn, Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse); Hypogammalglobulinemia; IgA Nephropathy; IgG4-related sclerosing disease; Immune thrombocytopenic purpura (ITP); Inclusion body myositis (IBM); Interstitial cystitis (IC); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, Ill; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosurn, Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC); Undifferentiated connective tissue disease (UCTD); Uveitis; Vasculitis; Vitiligo; or Vogt-Koyanagi-Harada Disease.

As another example, the autoimmune condition or immune disorder can be an autoinflammatory disease. The autoinflammatory can be Familial Mediterranean Fever (FMF), neonatal Onset Multisystem Inflammatory Disease (NOMID), Tumor Necrosis Factor Receptor-Associated Periodic Syndrome (TRAPS), Deficiency of the Interleukin-1 Receptor Antagonist (DIRA), Behcet's Disease, or Chronic Atypical Neutrophilic Dermatosis with Lipodystrophy and Elevated Temperature (CANDLE).

An infectious pathogen can be any infectious disease. For example, infections that can be treated with NK cells include, but are not limited to viral infections (e.g., cytomegalovirus, Epstein Barr virus, herpes simplex virus, human immunodeficiency virus), intracellular pathogens (e.g., Listeria monocytogenes), bacterial infections, and fungal infections. As another example, the treatment of an infectious disease can be for the treatment of any bacterial infection or viral infection, using a scFv that can recognize antigens, such as antigens on HIV infected cells. The infectious disease can be Acute Flaccid Myelitis (AFM); Anaplasmosis; Anthrax; Babesiosis; Botulism; Brucellosis; Campylobacteriosis, Carbapenem-resistant Infection (CRE/CRPA); Chancroid; Chikungunya Virus Infection (Chikungunya); Chlamydia; Ciguatera (Harmful Algae Blooms (HABs)); Clostridium Difficile Infection; Clostridium Perfringens (Epsilon Toxin); Coccidioidomycosis fungal infection (Valley fever); Creutzfeldt-Jacob Disease, transmissible spongiform encephalopathy (CJD); Cryptosporidiosis (Crypto); Cyclosporiasis; Dengue, 1,2,3,4 (Dengue Fever); Diphtheria; E. coli infection, Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection, Non-Polio (Non-Polio Enterovirus); Enterovirus Infection, D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpes; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease, Bacterial (Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague; Bubonic, Septicemic, Pneumonic (Plague); Pneumococcal Disease (Pneumonia); Poliomyelitis (Polio); Powassan; Psittacosis (Parrot Fever); Pthiriasis (Crabs; Pubic Lice Infestation); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection, Methicillin-resistant (MRSA); Staphylococcal Food Poisoning, Enterotoxin-B Poisoning (Staph Food Poisoning); Staphylococcal Infection, Vancomycin Intermediate (VISA); Staphylococcal Infection, Vancomycin Resistant (VRSA); Streptococcal Disease, Group A (invasive) (Strep A (invasive)); Streptococcal Disease, Group B (Strep-B); Streptococcal Toxic-Shock Syndrome, STSS, Toxic Shock (STSS, TSS); Syphilis, primary, secondary, early latent, late latent, congenital; Tetanus Infection, tetani (Lock Jaw); Trichomoniasis (Trichomonas infection); Trichonosis Infection (Trichinosis); Tuberculosis (TB); Tuberculosis (Latent) (LTBI); Tularemia (Rabbit fever); Typhoid Fever, Group D, Typhus; Vaginosis, bacterial (Yeast Infection); Vaping-Associated Lung Injury (e-Cigarette Associated Lung Injury); Varicella (Chickenpox); Vibrio cholerae (Cholera); Vibriosis (Vibrio); Viral Hemorrhagic Fever (Ebola, Lassa, Marburg); West Nile Virus; Yellow Fever; Yersenia (Yersinia); or Zika Virus Infection (Zika).

Administration

An aspect of the present disclosure provides for NK cells (e.g., CD8 deficient NK cells, CD8 deficient ML NK cells, modified NK cells, cytokine-activated NK cells) to be directly administered to a subject. The NK cells can be administered to the subject in an amount effective to enhance memory-like NK cell responses to target cells.

For example, the NK cells can be administered to the subject through, for example, an IV infusion, at an amount between about 0.05×10⁶ cells per kg patient body weight and about 100.0×10⁶ cells per kg patient body weight or 0.5×10⁶ cells per kg patient body weight and about 10.0×10⁶ cells per kg patient body weight.

As described herein clinical processing and treating patients with haplo/allogeneic ML NK cells or autologous ML NK cells can be performed using the ML NK cells as described herein. Apheresis (e.g., the removal of blood plasma from the body by the withdrawal of blood, its separation into plasma and cells, and the reintroduction of the cells) can be performed on the subject.

As described herein, the NK cells to be administered can be selected based on reduced CD8 expression. The NK cells can be purified and activated with IL-12/IL-15/IL-18 for about 12 to about 16 hours. The cells can be washed and infused into the patient at about 10⁷ cells/kg. In the haplo/allo setting the cells can be supported with rhIL-2 and in the autologous setting the cells can be supported with IL-15.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. Where the product is, for example, a biologic or cell therapy, the mode of administration will likely be via injection or infusion.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening for CD8 Inhibiting Agents

Also provided are methods for screening compounds as potential CD8 inhibiting agents.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to assays to measure CD8 expression levels in cells of a subject, CD8 inhibiting agents, reagents, or pharmaceutical compositions comprising CD8 inhibiting agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, or sterile saline, each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a responder, a non-responder, or a total failure (TF). A reference value can be used in place of a control or reference sample, which was previously obtained from a subject or a group of subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

### Additional Definitions

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The term “cancer” as used herein is meant to be synonymous with “tumor,” and refers to a disease of dysregulated and malignant cellular division/proliferation which can spread through the body. Cancer may refer to a hematologic malignancy or a solid tumor.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

The term “detecting” as used herein in relation to screening methods, is intended to be generally synonymous, and is used interchangeably with, the terms “measuring,” and means quantifying, using methods known in the art and appropriate to the goal, the amount (or, equivalently, level) of an analyte in a sample. The goal of the detecting/measuring may be, for example, comparison of the amount/level of analyte in the sample to another amount, for example, a mean amount in an appropriate population or an amount from a control subject or population, in order to enable clinically meaningful screening.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “enrich” as used herein in relation to NK cells means to concentrate or isolate for further analysis or use.

A “healthy donor,” as used herein, is one who does not have cancer.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease, for example as in the case of prevention of infection with a pathogen, or may involve prevention of disease progression, for example from an early stage of cancer to a later, more advanced stage. For example, prevention of a disease may not mean complete foreclosure of any effect related to the diseases at any level, but instead may mean prevention of the symptoms of a disease to a clinically significant or detectable level.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Multidimensional Analyses of Donor Memory-Like NK Cells Reveal New Associations with Response after Adoptive Immunotherapy for Leukemia

This example describes the discovery that the frequency of CD8α⁺ donor NK cells is negatively associated with AML patient outcomes after ML NK therapy (i.e., CD8a is associated with treatment failure).

Abstract

Natural killer (NK) cells are an emerging cancer cellular therapy and potent mediators of antitumor immunity. Cytokine-induced memory-like (ML) NK cellular therapy is safe and induces remissions in patients with acute myeloid leukemia (AML). However, the dynamic changes in phenotype that occur after NK-cell transfer that affect patient outcomes remain unclear. Here, it is reported, comprehensive multidimensional correlates from ML NK cell-treated patients with AML using mass cytometry. These data identify a unique in vivo differentiated ML NK-cell phenotype distinct from conventional NK cells. Moreover, the inhibitory receptor NKG2A is a dominant, transcriptionally induced checkpoint important for ML, but not conventional NK-cell responses to cancer. The frequency of CD8α⁺ donor NK cells is negatively associated with AML patient outcomes after ML NK therapy. Thus, elucidating the multidimensional dynamics of donor ML NK cells in vivo revealed critical factors important for clinical response, and new avenues to enhance NK-cell therapeutics.

Significance

Mass cytometry reveals an in vivo memory-like NK-cell phenotype, where NKG2A is a dominant checkpoint, and CD8a is associated with treatment failure after ML NK-cell therapy. These findings identify multiple avenues for optimizing ML NK-cell immunotherapy for cancer and define mechanisms important for ML NK-cell function.

Introduction

Natural killer (NK) cells are cytotoxic innate lymphocytes that are important for mediating antiviral host defense and responding to malignantly transformed cells (1). NK-cell activation is determined by the balance of signals received through germline DNA encoded activating, inhibitory, and cytokine receptors, which differs from T cells that rely on rearrangement of the T-cell receptor genes (2). Thus, NK cells are equipped to respond to a variety of malignant cells and have been investigated as a cellular immunotherapy for acute myeloid leukemia (AML), a clinically challenging blood cancer where the primary curative therapy is hematopoietic cell transplantation (HOT; refs. 3-5).

NK cellular immunotherapies are a nascent, promising, and safe alternative to T cells for cellular cancer immunotherapy (6, 7). Several types of NK-cell therapy have been shown to mediate antitumor responses in patients with AML without cytokine release syndrome (CRS) or immune cell-associated neurotoxicity syndrome (ICANS), which are frequent complications after chimeric antigen receptor T-cell immunotherapy approaches (4, 5, 7-9). However, the in vivo dynamic changes in donor NK cells that occur after transfer have not been extensively investigated, and both donor NK cell-intrinsic and host factors that contribute to treatment response and resistance are poorly understood.

Memory-like (ML) properties of NK cells after brief activation with the cytokines IL12, IL15, and IL18 followed by differentiation in vitro or in vivo in murine models and NSG mice have been previously disclosed (5, 10-12). In vitro differentiated ML NK cells have increased activating receptors, can ignore the rules of KIR-KIR ligand interactions, exhibit prolonged survival in NSG xenograft models, and have improved effector functions against a wide array of targets (5, 13, 14). The first-in-human clinical trial demonstrating that donor ML NK cells were safe, were detectable for several weeks after transfer, and induced complete remissions in patients with high-risk relapsed/refractory (rel/ref) AML was previously reported. However, not all patients responded, and the median duration of response was only a few months (5). The phenotypic changes in ML NK cells that occur during in vivo differentiation, and factors contributing to therapeutic response and resistance, were not explored and remain important questions in the field.

Here, mass cytometry was utilized to understand the dynamic changes that occur in ML NK cells during in vivo differentiation within patients with AML. It was discovered that ML NK cells are distinct from conventional and activated NK cells and have a unique, consistent, well-defined multidimensional signature. This multidimensional analysis was integrated with clinical results and identified NKG2A as the predominant checkpoint on ML NK cells, as well as unexpected characteristics of baseline donor NK cells that predict treatment failure.

Results

Mass Cytometry Distinguishes In Vivo Differentiated ML NK Cells

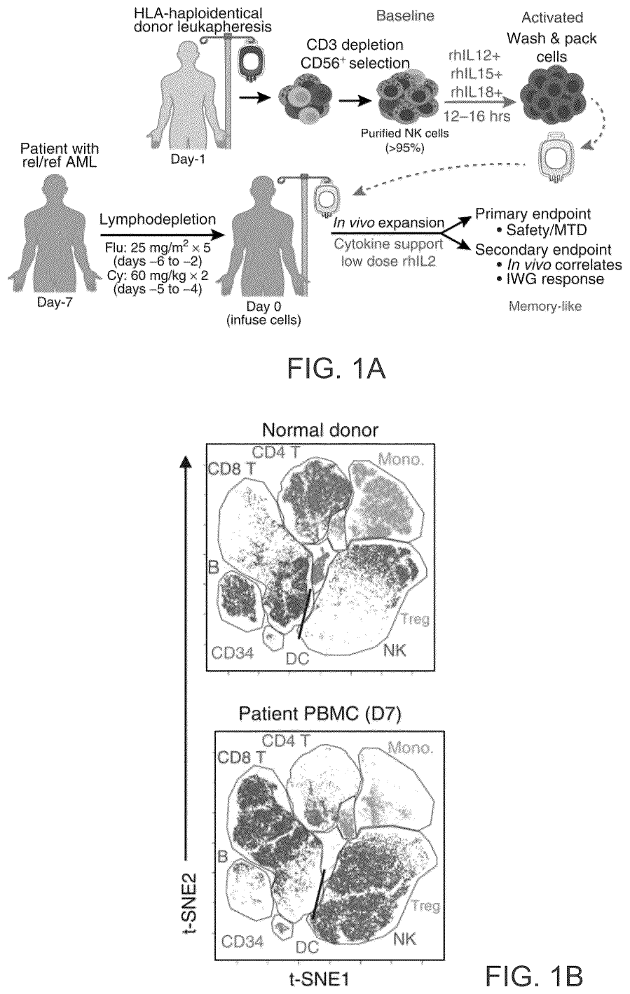

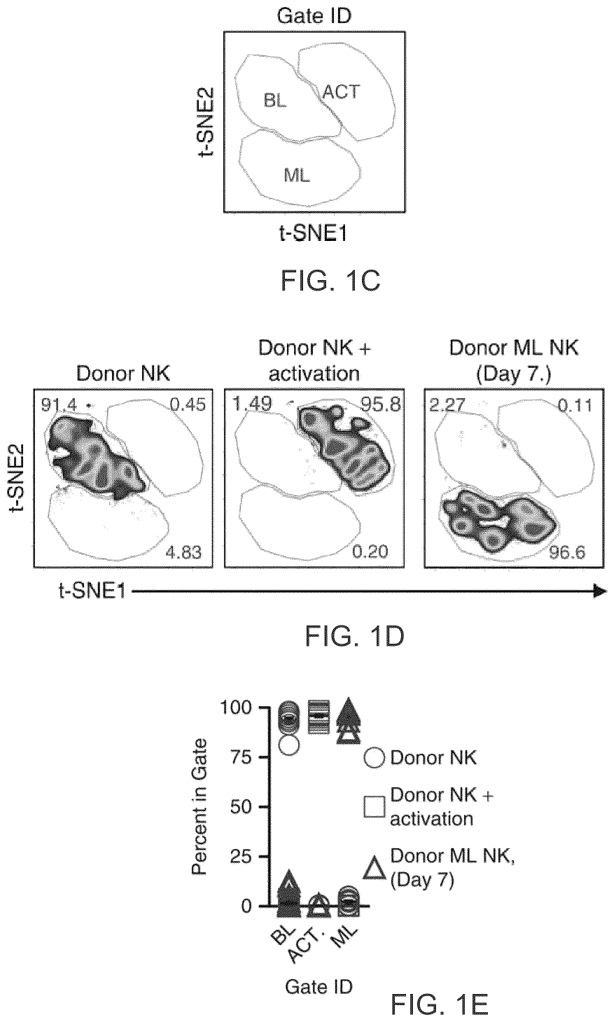

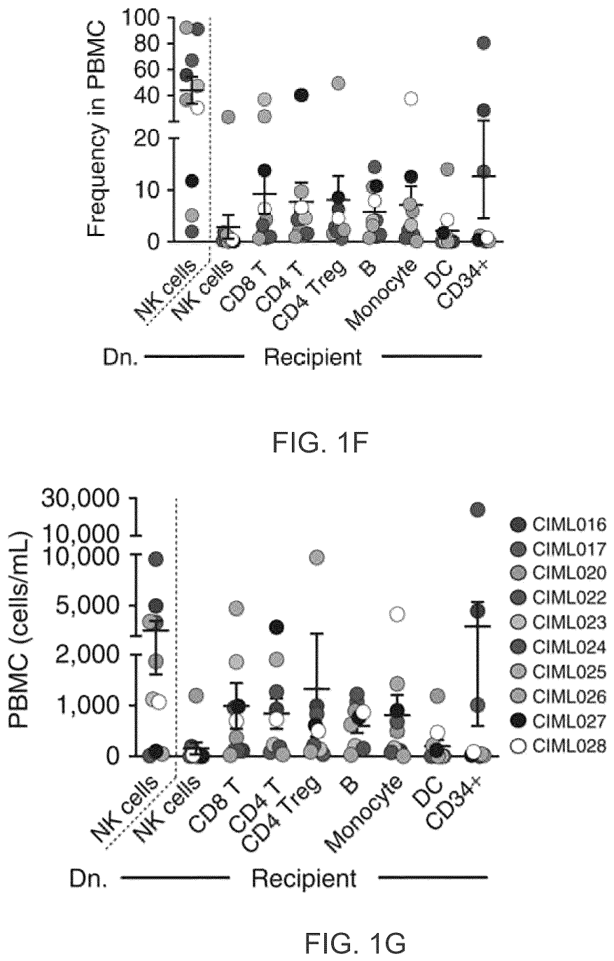

The initial report describing the dose-escalation cohort of the first-in-human trial using cytokine (IL12, IL15, and IL18) induced NK cells to treat patients with rel/ref AML demonstrated that donor ML NK cells expand and proliferate in vivo in patients with AML and result in complete remissions (FIG. 1A; ref. 5). The now-complete results of the phase I study evidence ML NK-cell therapy being well tolerated without CRS, graft-versus-host disease (GVHD), or neurotoxicity (TABLES 1-3). Among the 15 evaluable patients, 7 achieved complete response (CR; n=3) or complete response with incomplete count recovery (CRi; n=4) and 3 achieved a best response of morphologic leukemia-free state (MLFS) at day 14 by the IWG response criteria (15), for an overall International Working Group (IWG) response rate of 67% and a CR/CRi rate of 47% (TABLE 1).

TABLE 1
Patient summaries. Summary from all evaluable patients treated on study.

| | | | | | | Pre- | | | | |
|---|---|---|---|---|---|---|---|---|---|---|
| | | | | | | Treat- | | | | |
| | | | | | | ment | | | | |
| | | | | | | BM | No. | | | |
| | | Dose | | | | Blast | Prior | Best | LFS | OS |
| CyTOF? | Patient | Level | Gender | Age | Diagnosis | (%) | Therapies | Response | (days) | (days) |
| No | 001 | 1 | Male | 73 | AML | 16 | 2 | PD | 14 | 33 |
| No | 006 | 1 | Male | 70 | AML | 28 | 3 | PD | 14 | 60 |
| No | 007 | 1 | Male | 77 | AML | 47 | 1 | CR | 181 | 256 |
| No | 008 | 2 | Male | 76 | t-AML | 17 | 3 | PD | 28 | 75 |
| No | 009 | 2 | Female | 73 | AML | 80 | 3 | MLFS | 30 | 198 |
| No | 012 | 2 | Female | 71 | AML | 15 | 3 | CR | 104 | 338 |
| Yes | 016 | 3 | Male | 43 | AML | 78 | 7 | PD | 14 | 27 |
| Yes | 017 | 3 | Male | 63 | t-AML | 69 | 4 | MLFS | 31 | 43 |
| Yes | 020 | 3 | Male | 60 | AML | 13 | 1 | CRi | 77 | 77 |
| Yes | 022 | 3 | Female | 71 | AML | 90 | 1 | PD | 14 | 19 |
| Yes | 023 | 3 | Male | 83 | AML | 86 | 1 | CRi | 49 | 162 |
| Yes | 024 | 3 | Male | 73 | AML | 0 | 2 | MLFS | 29 | 29 |
| Yes | 025 | 2 | Male | 72 | t-AML | 2 | 1 | CR | 92* | 1260+ |
| Yes | 027 | 3 | Male | 76 | AML | 1 | 2 | CRi | 267 | 1170+ |
| Yes | 028 | 3 | Female | 69 | MDS | 5 | 1 | CRi | 91 | 390 |

BM, bone marrow;
AML, acute myeloid leukemia;
t-AML, treatment-related
AML; MDS, myelodysplastic syndrome;
PD, progressive disease;
CR, complete response;
MLFS, morphologic leukemia free state on day 14;
CRi, CR with incomplete count recovery;
LFS, Leukemia-free survival;
OS, overall survival.
*Leukemia-free survival censored at time of stem cell transplant.
For CIML026, see methods.
Dose level 1: 0.5 × 10⁶/kg, 2: 1 × 10⁶/kg, 3: 2-10 × 10⁶/kg.

TABLE 2
Toxicities considered possibly or probably related to
ML NK cell infusion, Grade ≥2 (n = 15). All adverse
events were treatment emergent and graded
according to the Common Terminology
Criteria for Adverse events (CTCAE), v4.

| Toxicity | Grade 2 | Grade 3 | Grade 4 |
|---|---|---|---|
| Increased | 1 | | |
| bilirubin | | | |
| Sinus tachycardia | 1 | | |
| Hypoxia | 1 | | |
| Dyspnea | 1 | | |
| Confusion | 1 | | |
| Lethargy | 1 | | |
| Chills | 1 | | |
| Anorexia | 1 | | |
| Fatigue | 1 | | |
| Alopecia | 1 | | |

TABLE 3
All toxicities ≥3, regardless of attribution, excepting
cytopenias and correctable electrolyte abnormalities
(n = 15). All adverse events were treatment
emergent and graded according to the CTCAE, v4.

| Toxicity | Grade 3 | Grade 4 | Grade 5 |
|---|---|---|---|
| Febrile | 13 | | |
| neutropenia | | | |
| Bacteremia | 3 | | |
| Candidemia | 1 | | |
| Sepsis | | 4 | |
| Skin infection | 5 | | |
| Urinary tract | 1 | | |
| infection | | | |
| Lung infection | 1 | | |
| Increased | 1 | | |
| bilirubin | | | |
| Increase liver | 2 | 2 | |
| enzymes | | | |
| Hyperglycemia | 3 | | |
| Hypoalbuminemia | 2 | | |
| Acidosis | 1 | | |
| Prolonged PTT | 1 | | |
| Troponin | 1 | | |
| increased | | | |
| Intracranial | 1 | | |
| hemorrhage | | | |
| Other bleeding | 4 | 1 | |
| Respiratory | 4 | | |
| failure | | | |
| Hypoxia | 3 | | |
| Shortness of | 1 | | |
| breath | | | |
| Pulmonary | 1 | | |
| edema | | | |
| Cardiopulmonary | | | 1 |
| arrest | | | |
| Hypotension | 4 | 1 | |
| Hypertension | 3 | | |
| Atrial fibrillation | 2 | | |
| Supraventricular | | | |
| tachycardia | | | |
| Generalized | 2 | | |
| weakness | | | |
| Headache | 1 | | |
| Acute kidney | 1 | | |
| injury | | | |
| Mucositis | | 1 | |
| Nausea | 2 | | |
| Edema | 2 | | |
| Multi-organ failure | | | 1 |
| Anorexia | 1 | | |
| Pain in extremity | 1 | | |
| Encephalopathy | | | 1 |

TABLE 4
NK cell phenotypic, functional and mass cytometry panels.
The metal isotope tag, marker name, antibody clone, source
and clustering usage are shown for the mass cytometry
phenotpyic (P), functional (F), or resistance (R) panels.
Clustering demarcates which channels were used for
generating the NK cell viSNE plots, and identifying
lymphocyte subsets (FlowSOM) in the phenotypic
panel (Lymph) or the resistance (Resist) panel.

| Tag | Antibody | Clone | Source | Panel | Clustering |
|---|---|---|---|---|---|
| 89 Y | CD45 | HI30 | Fluidigm | P/F/R | Lymph, |
| | | | | | Resist |
| 141 Pr | CD14 | M5E2 | BD | P/F/R | Lymph, |
| | | | Pharmingen* | | Resist |
| 142 Nd | CD19 | HIB19 | Fluidigm | P/F | Lymph |
| 143 Nd | KIR3DL1 | DX9 | R&D* | P/F | NK |
| 143 Nd | CD117 | 104D2 | Biolegend* | R | Resist |
| 144 Nd | FITC | FIT-22 | Fluidigm | P/F | NA |
| 144 Nd | CD19 | HIB19 | Biolegend* | R | Resist |
| 145 Nd | KIR2DS4 | FES172 | Beckman | P/F | NK |
| | | | Coulter* | | |
| 145 Nd | HLA-E | 3D12 | Biolegend* | R | NA |
| 146 Nd | KIR2DL1/ | EB6B | Beckman | P/F | NK |
| | 2DS1 | | Coulter* | | |
| 147 Sm | NKG2D | 1D11 | R&D* | P/F | NK |
| 147 Sm | CD15 | W6D3 | Biolegend* | R | Resist |
| 148 Nd | KIR2DL2/ | CH-L | BD | P/F/R | NK, |
| | 2DL3 | | Pharmingen* | | Resist |
| 148 Nd | KIR2DL1 | EB6B | Beckman | R | Resist |
| | | | Coulter* | | |
| 148 Nd | KIR3DL1 | X27 | Beckman | R | Resist |
| | | | Coulter* | | |
| 149 Sm | CD127 | AO19D5 | Fluidigm | P/R | Lymph, |
| | | | | | Resist |
| 149 Sm | T-Bet | 4B10 | BD | F | NA |
| | | | Pharmingen* | | |
| 150 Nd | CD4 | OKT4 | Miltenyi* | P/R | Lymph, |
| | | | | | Resist |
| 150 Nd | Mip1a | 93342 | R&D* | F | NA |
| 151 Eu | TRAIL | RIK-2 | Biolegend* | P | NK |
| 151 Eu | CD107a | H4A3 | Fluidigm | F | NA |
| 152 Sm | CD8 | SK1 | eBioscience* | P/R | Lymph, |
| | | | | | Resist |
| 152 Sm | TNF | Mab11 | Fluidigm | F | NA |
| 153 Eu | CD62L | DREG-56 | Fluidigm | P/F | NK |
| 154 Sm | KIR2DL5 | UP-R1 | Beckman | P/F | NK |
| | | | Coulter* | | |
| 155 Gd | CD27 | L128 | Fluidigm | P/F | NK |
| 156 Gd | PDL1/ | 29E.2A3 | Fluidigm | P | NA |
| | PDL2 | | | | |
| 158 Gd | CD137 | 4B4-1 | Fluidigm | P/F | NK |
| 159 Tb | NKG2C | 134591 | R&D* | P/F | NK |
| 160 Gd | CD69 | FN50 | Biolegend* | P/F | NK |
| 161 Dy | NKp30 | P30-15 | Biolegend* | P/F | NK |
| 162 Dy | KI-67 | B56 | Fluidigm | P | NK |
| 162 Dy | LAG3 | 11C3C65 | Biolegend* | F | NA |
| 162 Dy | FoxP3 | PCH101 | Fluidigm | R | Resist |
| 163 Dy | CD94 | DX22 | Biolegend* | P/F | NK |
| 163 Er | CD33 | WM53 | Fluidigm | R | Resist |
| 164 Dy | FoxP3 | PCH101 | Invitrogen* | P | NA |
| 164 Dy | Tim-3 | F38-2E2 | Biolegend* | F | NA |
| 165 Ho | CD16 | 3G8 | Fluidigm | P/F/R | Lymph, |
| | | | | | NK, |
| | | | | | ist |
| 166 Er | NKG2A | Z199 | Beckman | P/F/R | NK |
| | | | Coulter* | | |
| 167 Er | NKp44 | P44-8 | Biolegend* | P/F | NK |
| 168 Er | DNAM1 | DX11 | Miltenyi* | P | NK |
| 168 Er | IFN- | B27 | Fluidigm | F | NA |
| 169 Tm | CD25 | 2A3 | Fluidigm | P/R | Lymph, |
| | | | | | NK, |
| | | | | | Resist |
| 169 Tm | Eomes | WD1928 | Invitrogen* | F | NA |
| 170 Er | CD34 | 581 | Invitrogen* | P/F/R | Lymph, |
| | | | | | Resist |
| 171 Yb | Granzyme | GB11 | Fluidigm | P | NK |
| | B | | | | |
| 171 Yb | PD-1 | EbioJ105 | Invitrogen* | F | NA |
| 172 Yb | CD57 | HCD57 | Fluidigm | P/F | NK |
| 173 Yb | CD3 | UCHT1 | BD | P/F/R | Lymph, |
| | | | Pharmingen* | | Resist |
| 174 Yb | NKp46 | 9E2 | R&D* | P/F | NK |
| 174 Yb | HLA-DR | L243 | Fluidigm | R | Resist |
| 175 Lu | Perforin | B-D48 | Fluidigm | P/F | NK |
| 176 Yb | CD56 | NCAM16.2 | Fluidigm | P/F/R | Lymph, |
| | | | | | NK, |
| | | | | | Resist |
| 209 Bi | CD11b | 209Bi | Fluidigm | P/F/R | Lymph, |
| | | | | | NK, |
| | | | | | ist |

*The asterisk included after the source indicates antibodies that were custom-conjugated using Fluidigm antibody labeling kits, per manufacturer's instructions.
NA, not applicable.

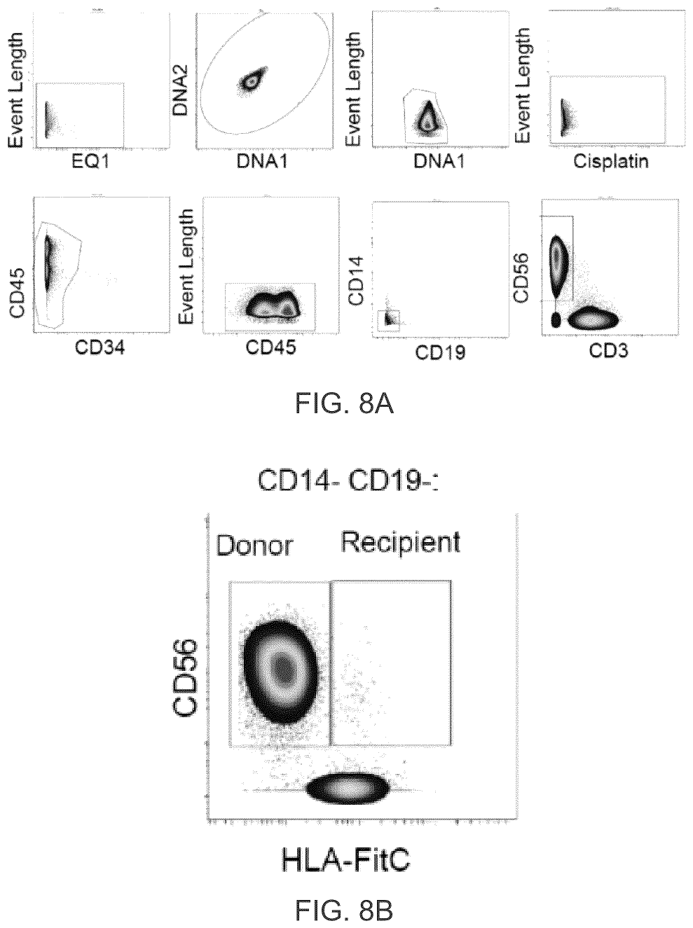

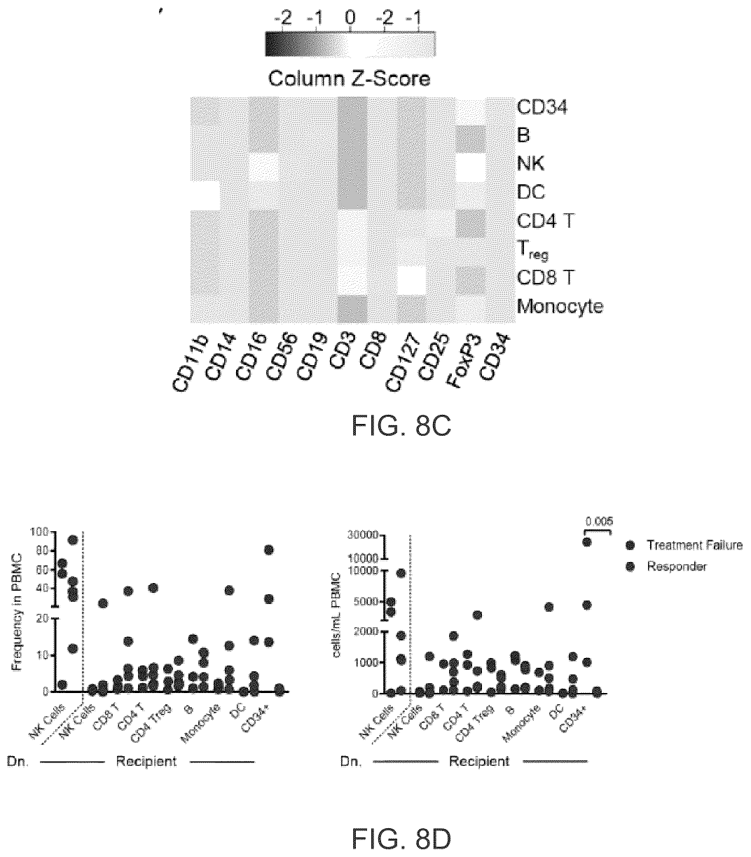

Donor NK cells were detected in both patient peripheral blood (PB) and bone marrow (BM) by flow cytometry, with peak expansion occurring 7 to 14 days post-NK cell infusion for patients at all dose levels (5). It was hypothesized that in vivo differentiated ML NK cells are distinct from baseline NK cells and NK cells acutely activated with cytokines. To test this, patient PB and BM were analyzed using a 37-marker NK-cell mass cytometry panel (TABLE 4; FIG. 8A and FIG. 8B) and major immune cell subsets were identified using FIowSOM (ref. 16; FIG. 1B, FIG. 8C), including NK cells. Donor NK cells in recipient peripheral blood mononuclear cells (PBMC) were identified using donor- and recipient-specific HLA mAbs (FIG. 8B). Donor NK cells were compared at the time of initial isolation (baseline), following 12- to 16-hour cytokine activation (immediately prior to infusion), and within patient PB or BM mononuclear cells (when available) 7 days following NK-cell infusion using t-SNE-based analysis (viSNE). On the basis of 25 markers, baseline (BL), cytokine-activated (ACT), and ML NK cells are distinct (FIGS. 1C and 1D, TABLE 4) as indicated by discrete islands within the viSNE maps. These distinctions are consistent across the 11 available patients assessed by mass cytometry at this time point (TABLE 1; FIG. 1E; P>0.001 as determined by two-way ANOVA, see Methods). For a majority of the patients, donor NK cells are the main lymphocyte subset by frequency and total numbers (FIG. 1F and FIG. 1G), confirming initial flow cytometry results on a subset of patients (5). Within the dose level 3 cohort (2-10×10⁶ ML NK cells/kg), a significant association between NK-cell frequency or absolute cell numbers in PB at day 7 and clinical response was not detected, although this study was not powered for this correlative endpoint (FIG. 8D). Similarly, an association between regulatory T (Treg) cell numbers or frequency and patient responses as not detected, different from other types of NK-cell therapy (17). Total circulating CD34⁺ cells (expressed on most AML) were significantly negatively associated with response, as expected (FIG. 8D).

In Vivo Differentiated ML NK Cells are Phenotypically Distinct

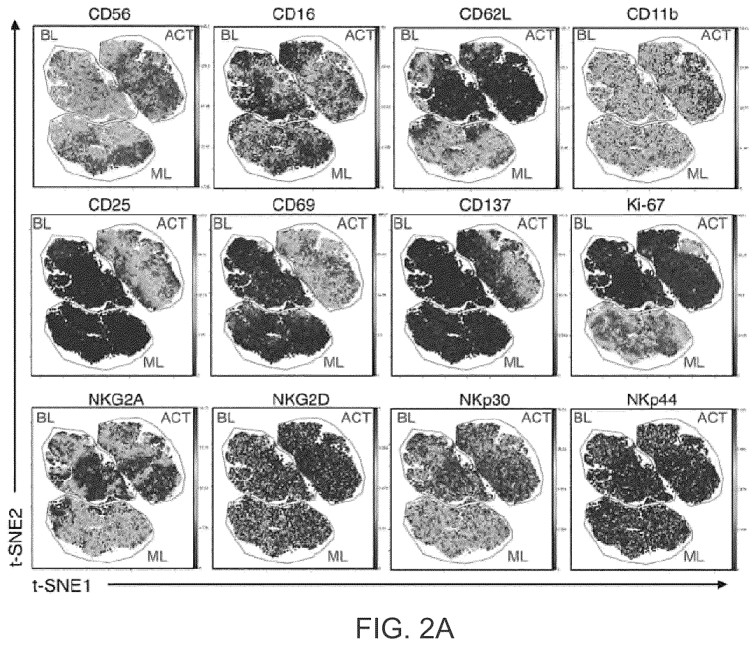

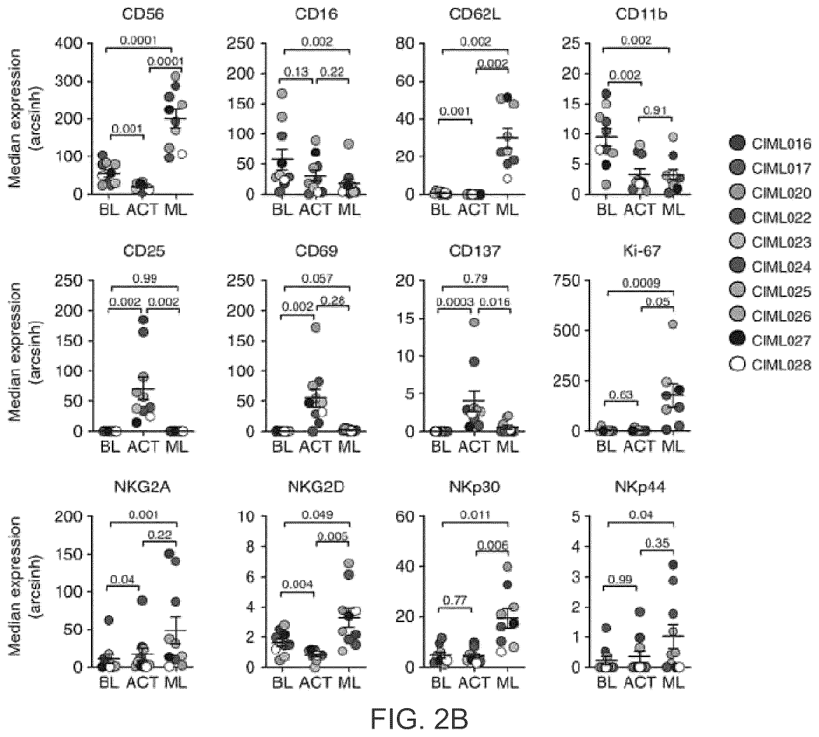

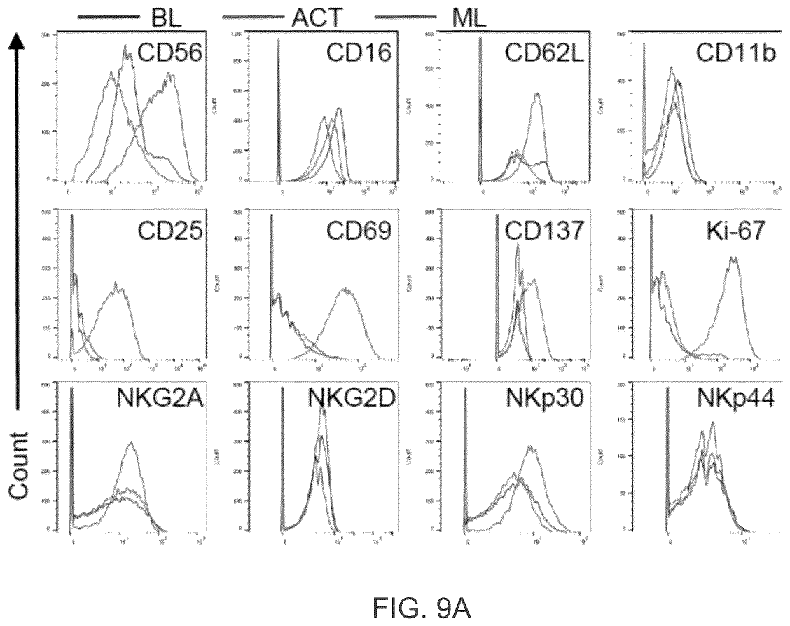

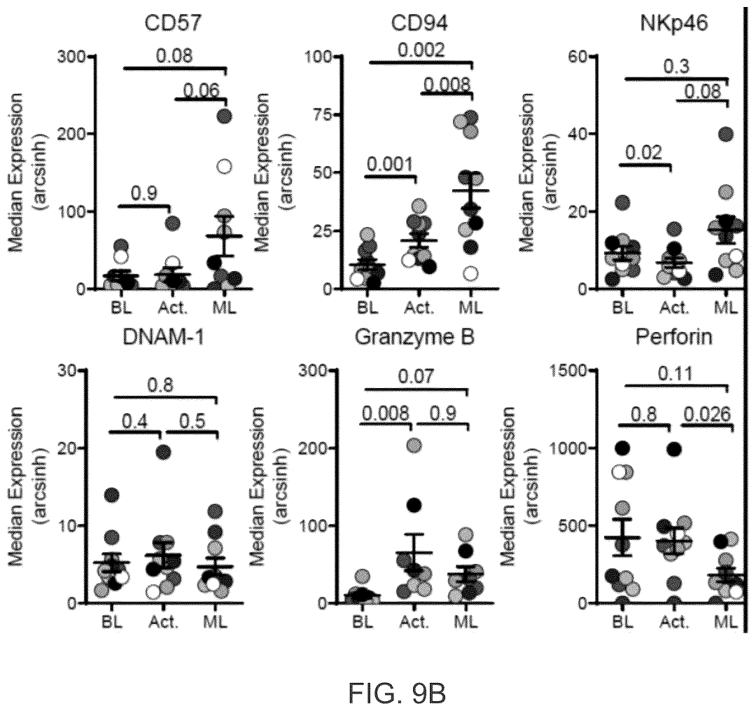

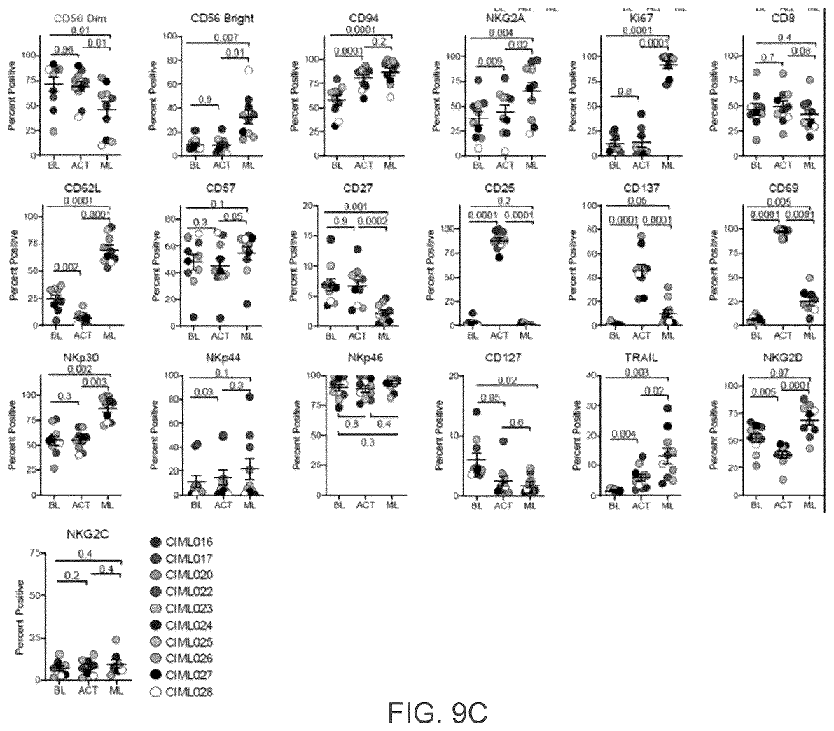

On the basis of in vitro studies, it was hypothesized that ML NK cells could be distinguishable from conventional NK cells by examining a large number of cell surface and intracellular markers. Using the median expression of markers for each BL, ACT, and ML NK-cell subset defined on the basis of t-SNE analysis (FIG. 1), the markers significantly associated with each NK-cell type were identified. ACT NK cells were defined by significantly decreased CD56 and increased CD25, CD69, and CD137, which are well-defined markers of acute NK-cell activation, and consistent with the lab's in vitro reports (FIGS. 2A and B; FIG. 9A-FIG. 9B; refs. 5, 13). ML NK cells were defined by significantly increased CD56, Ki-67, NKG2A, and activating receptors NKG2D, NKp30, and NKp44 (FIG. 2A and FIG. 2B; FIG. 9A). In addition, modest decreases in the median expression of CD16 and CD11 b were observed (FIG. 2A and FIG. 2B). ML NK cells expressed CD16 following in vivo differentiation (median percent positive 69±16%), consistent with prior studies demonstrating ML NK cells have enhanced antibody-dependent cellular cytotoxicity (14). Increased frequency of TRAIL, CD69, CD62L, NKG2A, and NKp30-positive NK cells were observed in ML NK cells compared with both ACT and BL, whereas the frequencies of CD27⁺ and CD127⁺ NK cells were reduced (FIG. 9C). Finally, unlike in vitro differentiated ML NK cells, in vivo differentiated ML NK cells did not express CD25 (IL2Rα, FIG. 2A and FIG. 2B; FIG. 90, ref. 5). It was postulated this may be due to in vivo ligation by low-dose IL2 used to support ML NK cells.

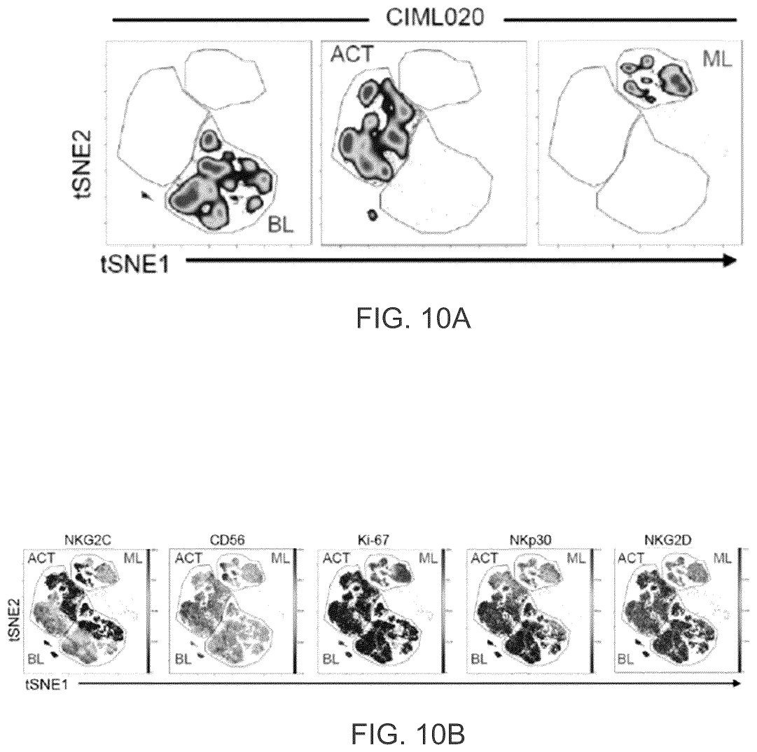

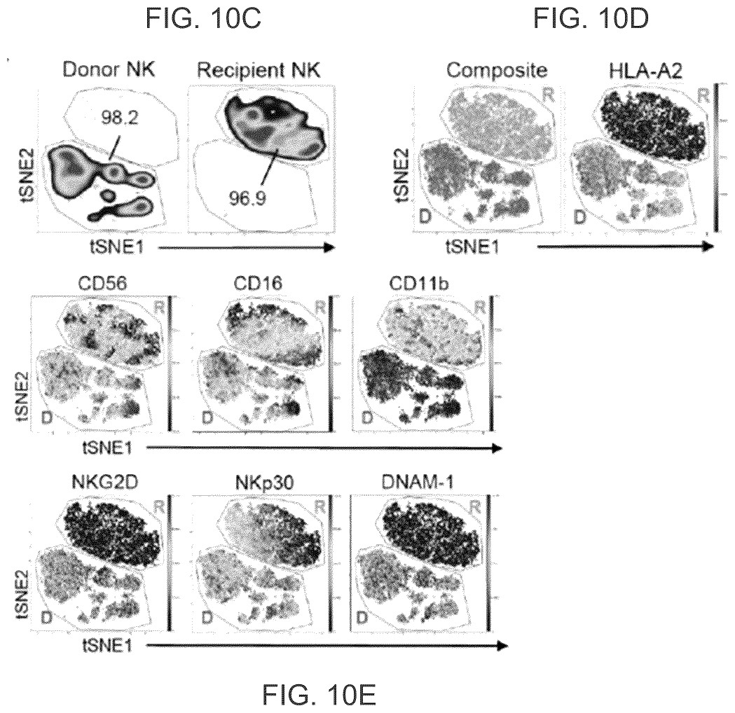

In one case, a cytomegalovirus seropositive donor's NK cells were predominantly CD57⁺ NKG2C⁺, which are presumably comprising adaptive NK cells (18, 19). Using mass cytometry, it was determined that adaptive NK cells could also differentiate into ML NK cells in vivo, as the cells exhibited an ML NK-cell signature, including increased CD56 and activating receptor expression (CIML020, FIG. 9B and FIG. 10A and FIG. 10B). However, the fraction of CD57⁺ NKG2C⁺ cells remained constant at BL, ACT, and following in vivo ML NK-cell differentiation, suggesting that the presence of adaptive markers and biology did not affect ML NK-cell differentiation. NKG2C expression was modest on the remaining donor NK cells and was not altered by in vivo ML NK-cell differentiation (FIG. 9C).

Because this patient had sufficient donor and recipient NK cells for advanced analysis, PB NK cells at D7 were examined (FIG. 10C-FIG. 10E). Using the same analysis approach (FIG. 2A and FIG. 2B), donor and recipient NK cells were compared using viSNE and represent distinct populations (FIG. 10C-FIG. 10E). The separation of these populations is confirmed by HLA-A2 staining (FIG. 10D). Finally, the donor ML NK cells demonstrate the consistent ML phenotype, whereas the recipient NK cells do not have increased NKG2A, are CD11b⁺, and have lower activating receptor expression compared with donor ML NK cells (FIG. 10E). These analyses were not possible for additional patients due to a paucity of recipient NK cells present at D7, but support that ML NK cells are phenotypically distinct from baseline NK cells.

ML NK Cells are Similar Between Patient BM and Blood

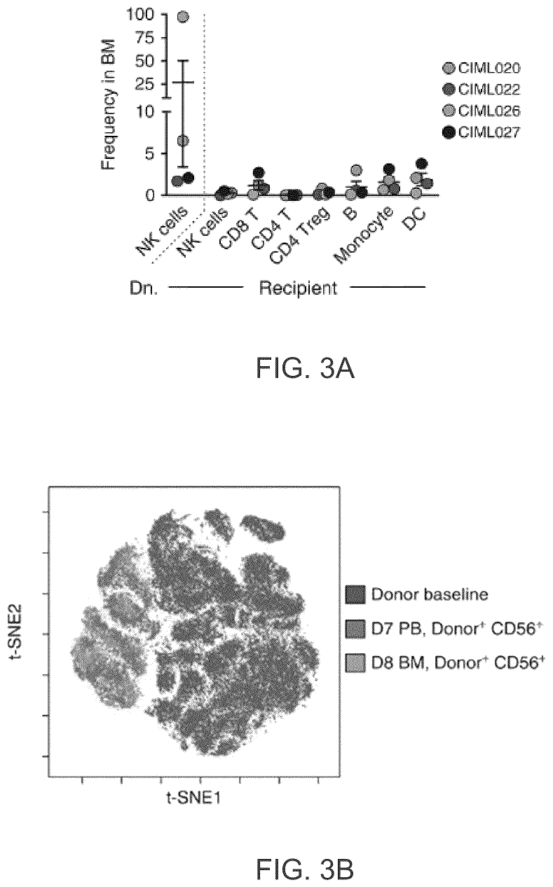

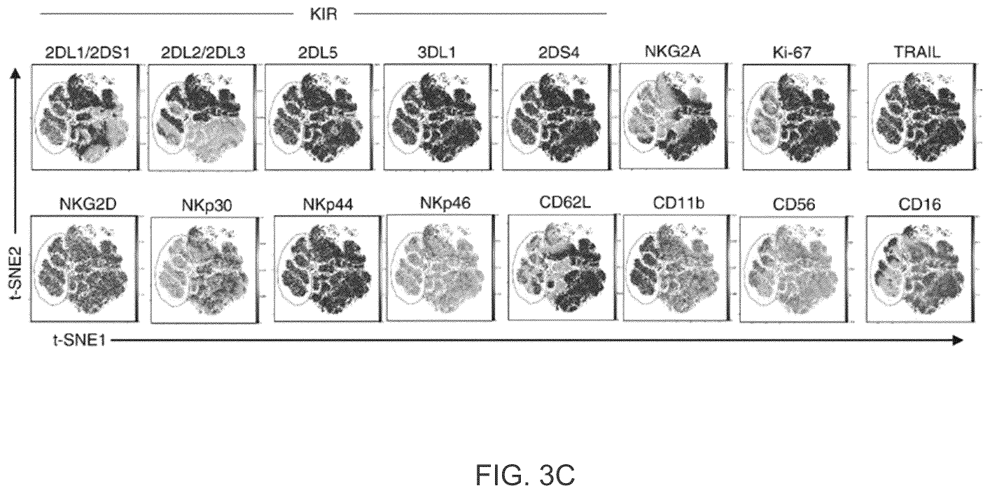

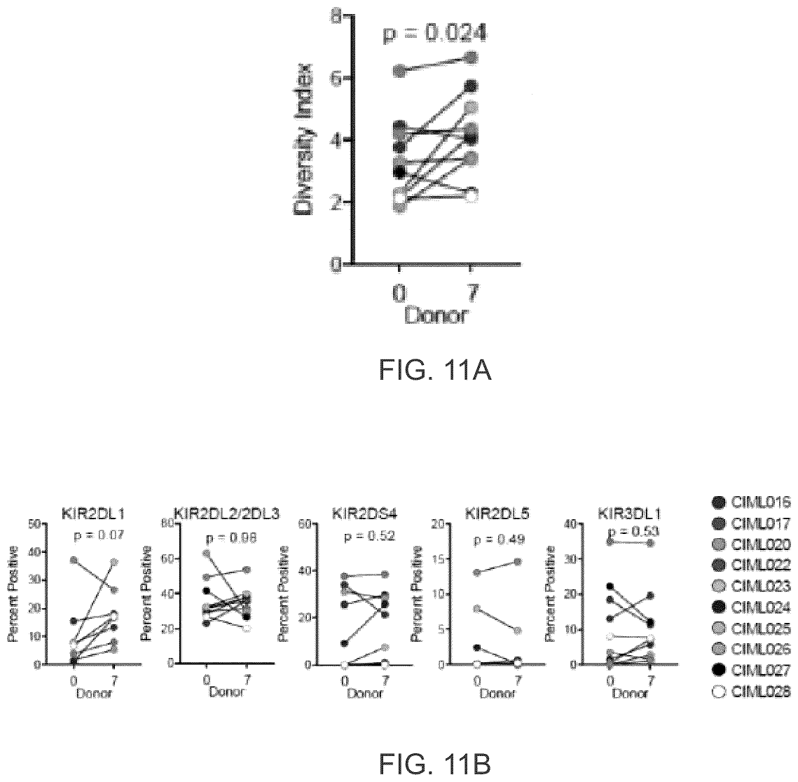

Because AML routinely involves the BM as a unique AML microenvironment, patient BM mononuclear cells (BMMC) were also examined and compared with PB ML NK cells using mass cytometry at day 7 or 8 post-infusion. Consistent with the lab's previous reports, donor NK cells trafficked to the BM and represented the major population observed in this tissue for most patients assessed (FIG. 3A; TABLE 4; ref. 5). Using t-SNE analysis (FIG. 1 and FIG. 2), PB and BM donor NK cells had a similar multidimensional phenotype when compared to each other, but were again distinct from BL NK cells (FIG. 3B and FIG. 3C). Although median expression of most markers assessed was similar between BM and PB NK cells, BM donor—positive NK cells were significantly reduced in NKp46 (PB 18.66±2.14 SEM v BM 7.66±2.56, P=0.006), potentially indicating downregulation after interaction with AML blasts. KIR expression and KIR diversity on in vitro differentiated ML NK cells did not vary (5). To understand how KIR repertoire was altered by in vivo donor ML differentiation, KIR diversity on donor BL and in vivo differentiated ML NK cells were compared (5, 20). If a particular subset of KIR-expressing cells had a proliferative advantage in vivo, it was expected that KIR diversity would decrease. However, here it was observed that KIR diversity modestly increased, without significant changes in any single KIR (FIG. 11A and FIG. 11B).

Donor ML NK Cells Differentiated in Patients are Polyfunctional Ex Vivo

For a subset of patients with adequate cell numbers, ex vivo functional responses against K562 leukemia targets were examined using mass cytometry. Freshly isolated PBMCs were coincubated with K562 cells for 6 hours; degranulation (CD107a), cytokine production (IFNγ, TNF) and chemokine production (MIP1α) were measured using the functional mass cytometry panel (FIG. 4A; TABLE 4). When cocultured with leukemia targets, donor ML NK cells produced significantly increased IFNγ and MIP1α, compared with unstimulated NK cells (FIG. 4B). When polyfunctional responses were assessed, it was observed 46% to 99% of donor NK cells are producing at least 1 cytokine/chemokine in response to tumor triggering (FIG. 4C). Previous work reported that ML differentiation improved effector functions of unlicensed NK cells (14). To investigate whether in vivo ML differentiation affects unlicensed NK-cell functionality, the effector functions of single KIR⁺ donor NK cells in response to K562 was examined. NK cells that were unlicensed in the donor would be expected to produce fewer effector molecules compared with licensed NK cells. In most cases, the unlicensed KIR-expressing ML NK-cell subsets produced IFNγ, TNF, and MIP1α, and expressed CD107a similarly to the licensed KIR-expressing ML NK-cell subsets (FIG. 4D), consistent with the idea that unlicensed donor ML NK cells have enhanced function following ML differentiation in vivo. However, interpreting these in vivo data is complicated by the fact that each KIR is predicted to be licensed in the patient (FIG. 4D), leaving open the possibility that a licensing event also occurred after in vivo transfer.

NKG2A is a Dominant Inhibitory Checkpoint on ML NK Cells

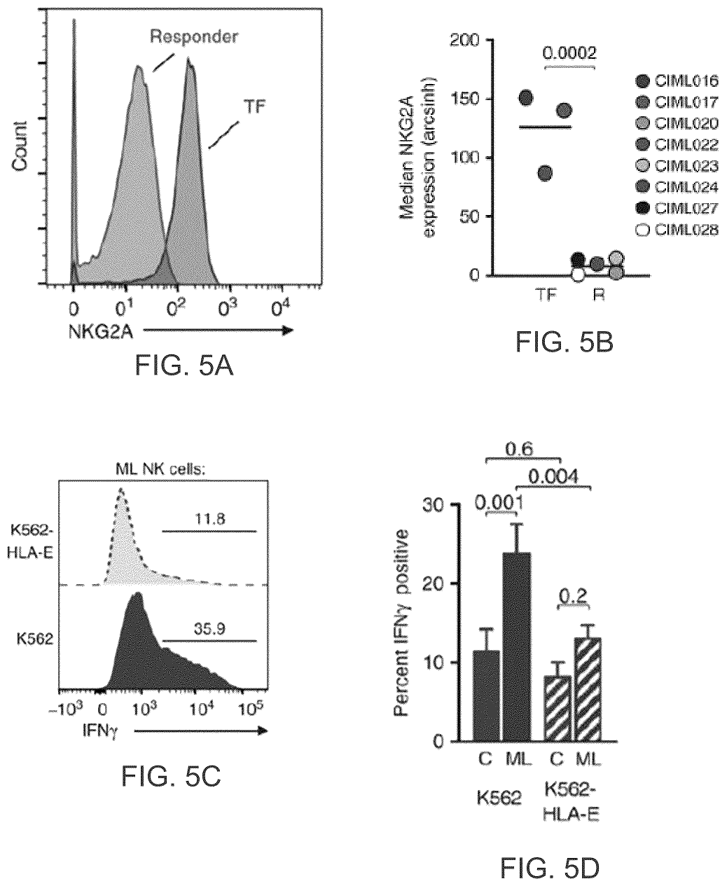

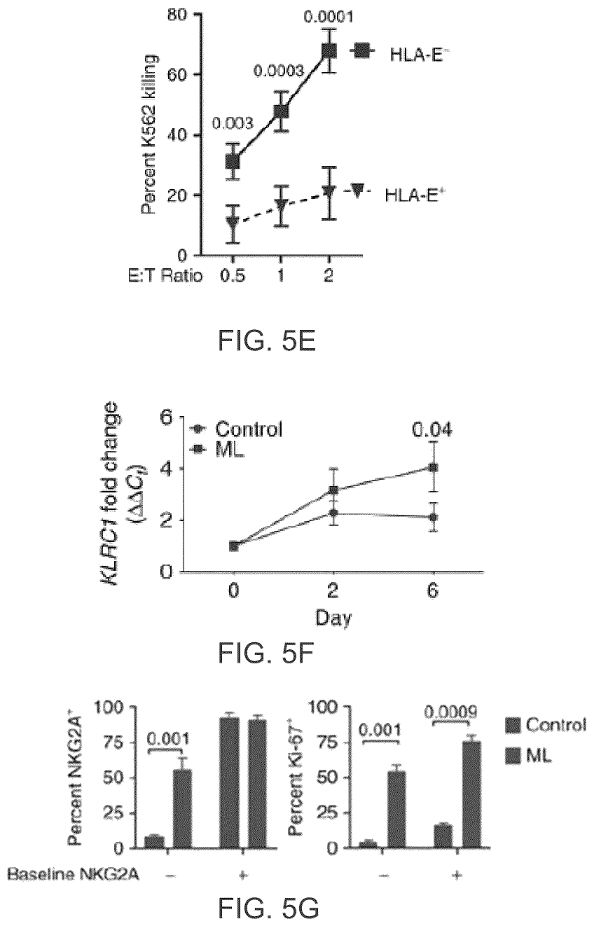

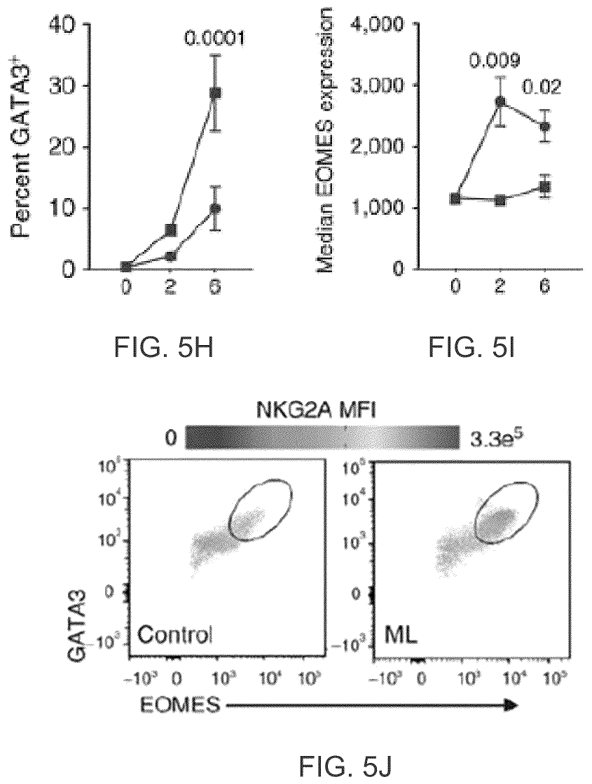

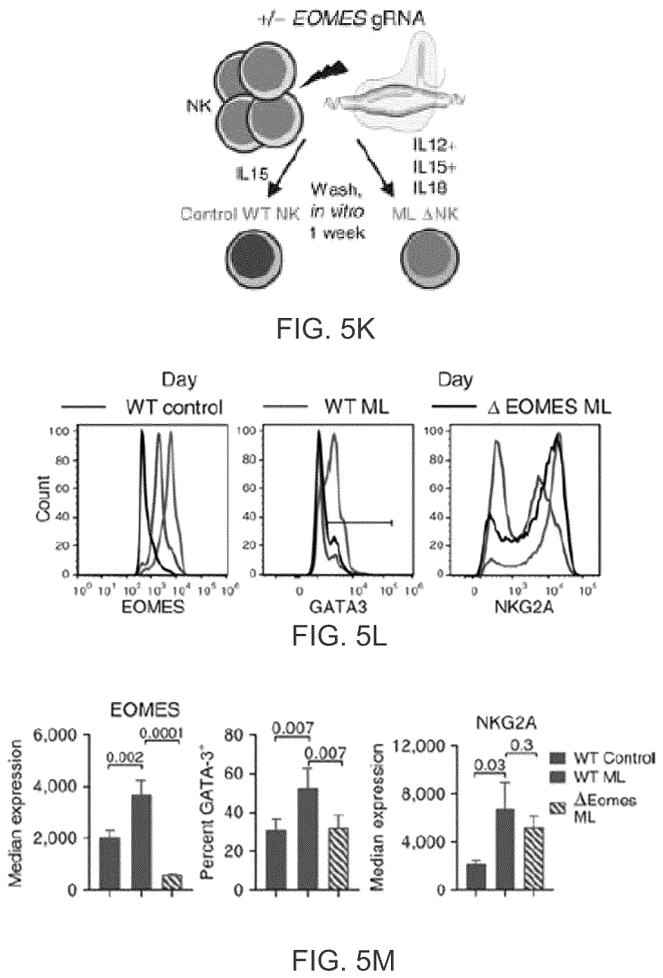

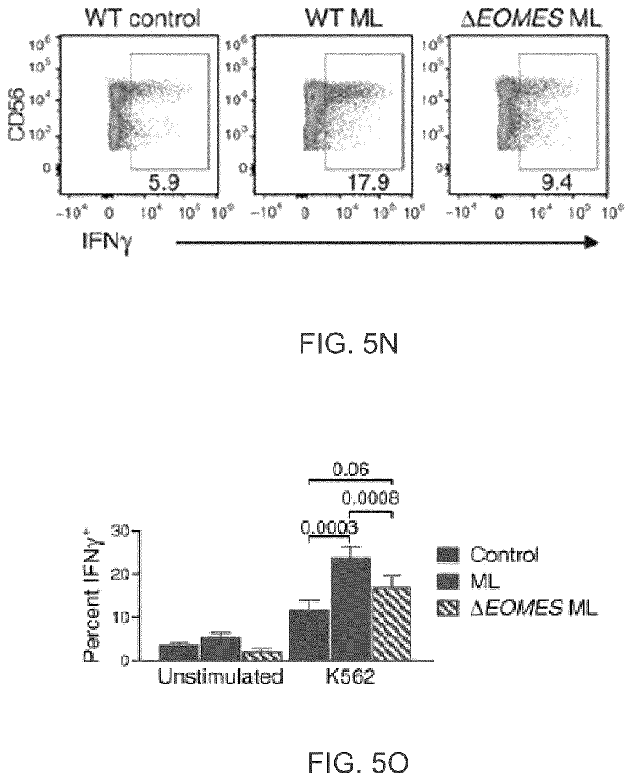

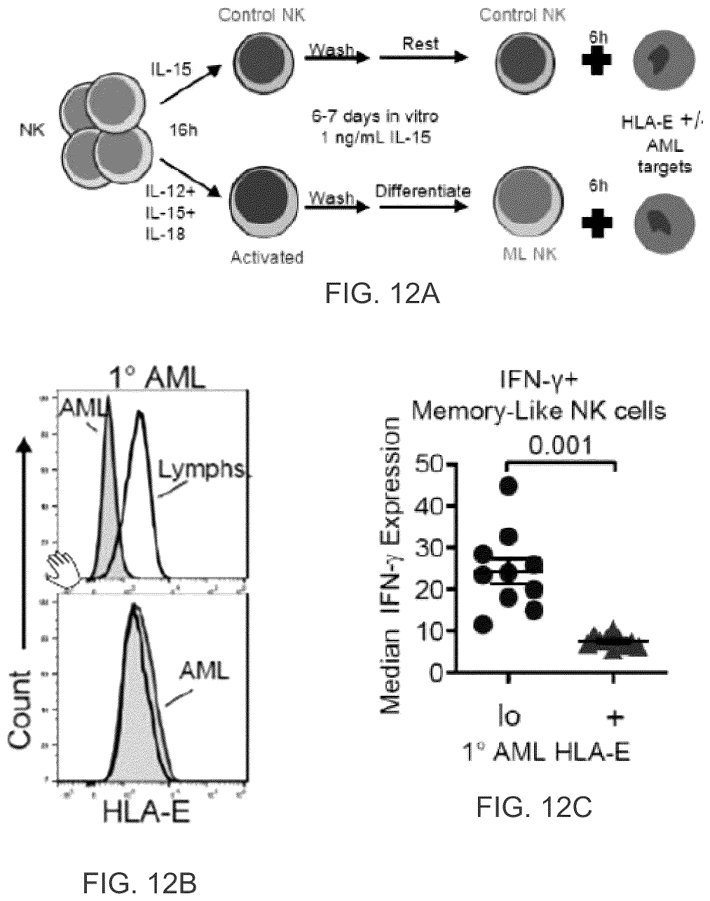

To determine whether any markers were associated with treatment failure (TF), the evaluable dose level of 3 patients with available cytometry by time of flight data [3 TF, 5 responders (R)] using Citrus was assessed (21). Citrus identified that NKG2A median expression on donor ML NK cells in the PB at D7 was significantly associated with TF (SAM, FDR<0.01). Indeed, NKG2A median expression was significantly increased on donor NK cells transferred into patients with subsequent TF compared with those who achieved an IWG response (FIGS. 5A and B). In contrast, NKG2A expression on baseline donor NK cells in this study was 8% to 76% with a median of 38% expression (FIG. 9C), and was not associated with clinical outcomes. NKG2A is an inhibitory receptor that interacts with the nonclassic MHC-I molecule HLA-E (22, 23). To determine whether NKG2A inhibits ML NK-cell responses, control or ML NK cells from normal donor PBMCs were generated in vitro (FIG. 12A) and stimulated with HLA-E^{lo} or HLA-E⁺ primary AML, and analyzed for IFNγ production (FIG. 12B and FIG. 12C). ML NK cells responding to HLA-E⁺ primary AML produce less IFNγ on a per-cell basis than ML NK cells triggered with HLA-E^{lo} tumor targets, consistent with the in vivo association with TF. Next, K562 were generated that overexpress HLA-E to trigger control or ML NK cells (FIG. 5C and FIG. 5D). In these assays, ML NK cells produced more IFNγ than control NK cells in response to K562, as expected. However, ML NK cells, but not control NK cells, demonstrated reduced IFNγ production when stimulated with HLA-E⁺ K562 targets compared with HLA-E—negative targets (FIG. 5D). Indeed, the enhanced functionality typically observed in ML NK cells was completely abrogated when HLA-E was present on the targets (FIG. 5D). To determine whether NKG2A interactions with HLA-E also inhibited target killing, ML NK cells were incubated with HLA-E⁺ or HLA-E⁻ K562 targets, and specific killing was measured in a flow-based killing assay (13). ML NK cells demonstrated a significantly reduced ability to kill HLA-E⁺ K562 targets compared with HLA-E⁻ K562 (FIG. 5E).

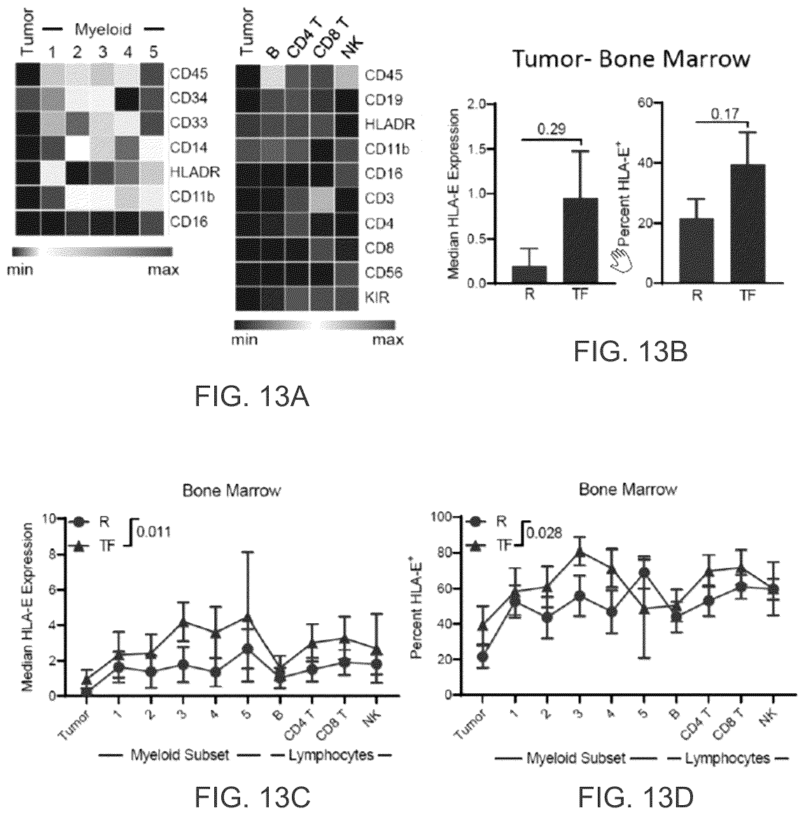

Patient BM samples obtained prior to treatment of study were examined by mass cytometry, and unbiased FIowSOM was used to define cell populations within the tumor microenvironment (FIG. 13A; TABLE 4). Using this approach, HLA-E expression on these subsets was compared between responders and TF (FIG. 13B-FIG. 13D). Although HLA-E expression on AML blasts was not associated with clinical outcomes, treatment failure was associated with increased HLA-E expression on mononuclear cells within the bone marrow (FIG. 13B-FIG. 13D). These data suggest that increased NKG2A expression and HLA-E expression in the bone marrow negatively affected ML NK-cell responses in vivo.

NKG2A is Transcriptionally Induced in ML NK Cells

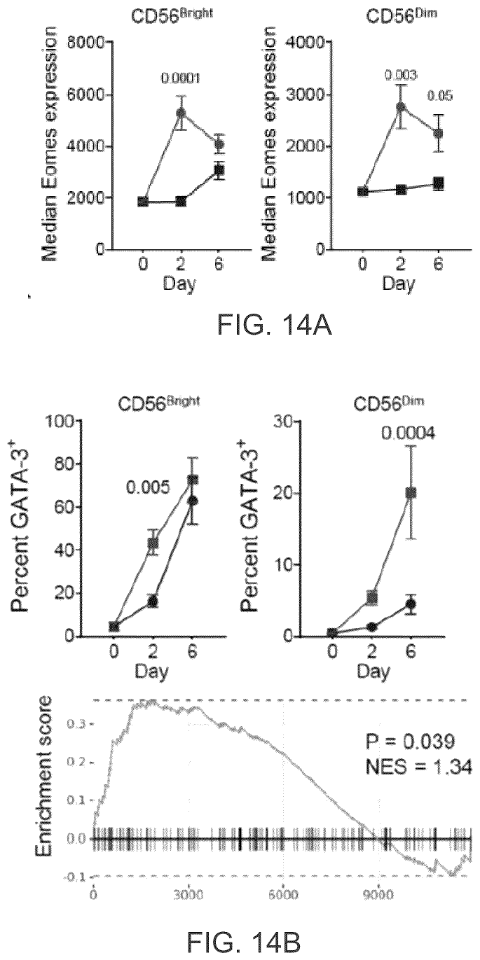

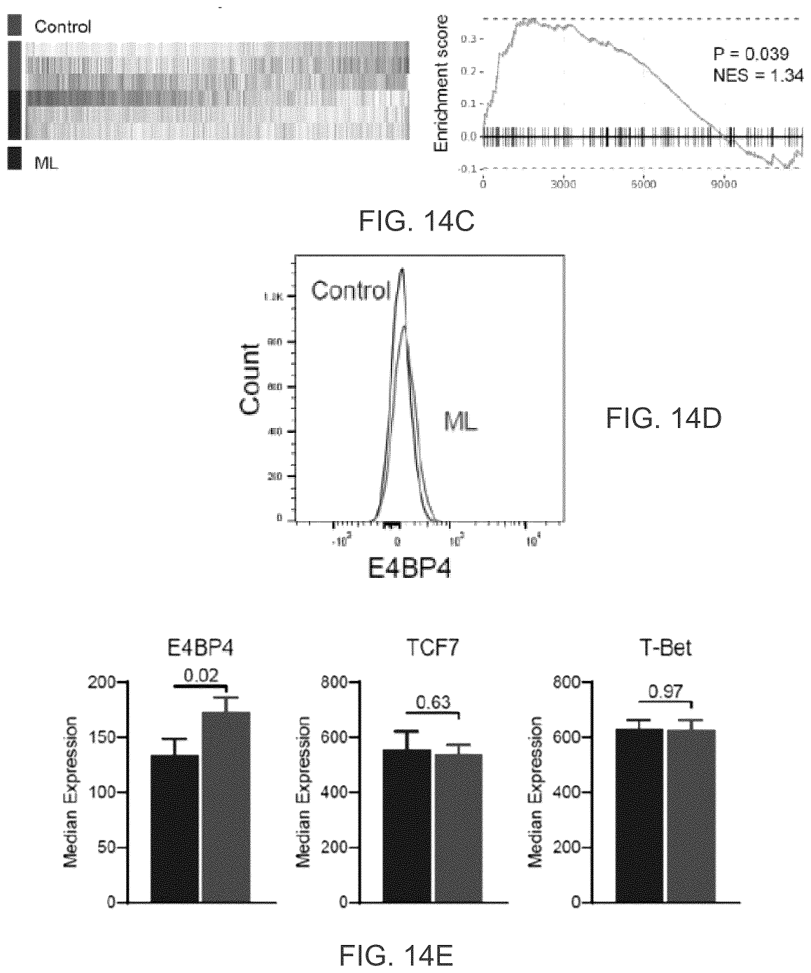

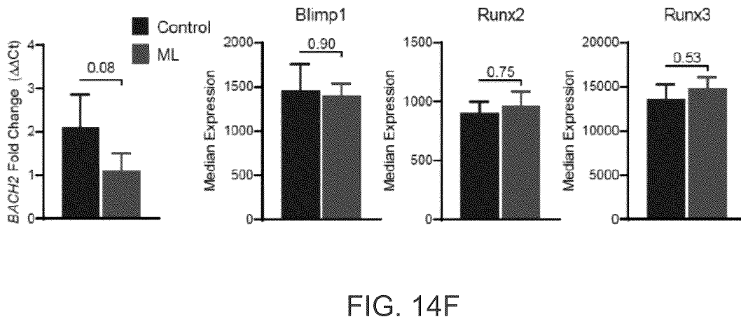

To understand the mechanisms underlying this increased NKG2A expression by ML NK cells, qRT-PCR was performed for KLRC1 (the gene that encodes NKG2A) on in vitro control or differentiated ML NK cells over time (FIG. 5F). ML NK cells, but not control treated NK cells, induced KLRC1 mRNA. To determine whether NKG2A expression was occurring de novo, CD56d′m CD16⁺ NKG2A+ and NKG2A⁻ cells were sorted and examined NKG2A and Ki-67 expression on control and ML NK cells after 7 days in vitro (FIG. 5G). Here, NKG2A-negative cells induced NKG2A expression after ML NK-cell differentiation, but not control incubation. In addition, Ki-67 is increased in both NKG2A⁺ and NKG2A⁻ NK ML NK-cell subsets, but to a greater extent in NKG2A⁺ ML NK cells (FIG. 5G). These data suggest that both an expansion of NKG2A⁺ NK cells and de novo NKG2A upregulation are responsible for increased NKG2A during ML NK-cell differentiation. Previous reports have implicated GATA3 as a transcription factor that regulates NKG2A expression (24). Indeed, the frequency of GATA3⁺ NK cells is specifically increased in ML NK cells, compared with control NK cells (FIG. 5H). In addition to GATA3, the transcription factor EOMES was increased in ML NK cells compared with control (FIG. 5I). Furthermore, EOMES and GATA3 coexpression corresponded with the NKG2A^{hi} cells, suggesting these transcription factors are important for the NKG2A upregulation within ML NK cells (FIG. 5J). Finally, GATA3 and EOMES are increased in both CD56^{bright} and CD56^{dim} subsets in response to ML differentiation (FIG. 14A and FIG. 14B). Gene set enrichment analysis (GSEA) comparing expressed genes in control and ML NK cells revealed ML NK cells were significantly enriched in GATA3 target genes compared with control NK cells (FIG. 14C). In similar assays, E4BP4, TCF7, TBET, BLIMP1, RUNX2, and RUNX3 median expression were similar between control and ML NK cells (FIG. 14D and FIG. 14E). BACH2 mRNA expression was also similar between control and ML NK cells (FIG. 14F). Together, these data support the lab's previous findings that CD56^{bright} and CD56^{dim} NK cells both have the ability to differentiate into ML NK cells and demonstrate GATA3 and EOMES as specifically regulated by ML NK-cell differentiation (5, 10).

Eomes Regulates GATA3 and Promotes ML NK Cell-Enhanced Responses to Leukemia Targets

Because EOMES has a well-defined role in promoting T-cell memory (25), it was hypothesized that it would be involved in memory formation in cytokine-activated NK cells. CRISPR/Cas9 was used to delete EOMES prior to ML differentiation (FIG. 5K-FIG. 5O). EOMES was reduced in EOMES ML NK cells compared with wild-type (WT) control and WT ML NK cells (FIG. 5L and FIG. 5M). The increase in GATA3 frequency during ML NK-cell differentiation was abrogated by loss of EOMES (FIG. 5L-FIG. 5M). Finally, increased IFNγ responses by ML NK cells compared with control NK cells was also partially abrogated by EOMES deletion (FIG. 5N and FIG. 5O), implicating EOMES as a critical transcription factor for ML NK-cell differentiation.

NKG2A Checkpoint Blockade or Elimination Restores ML NK-Cell Responses to AML

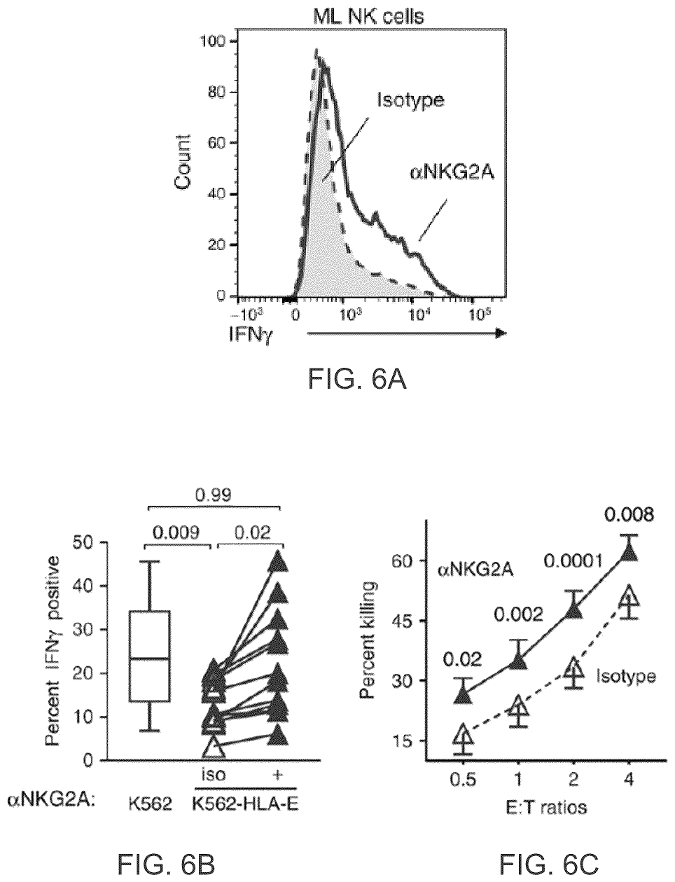

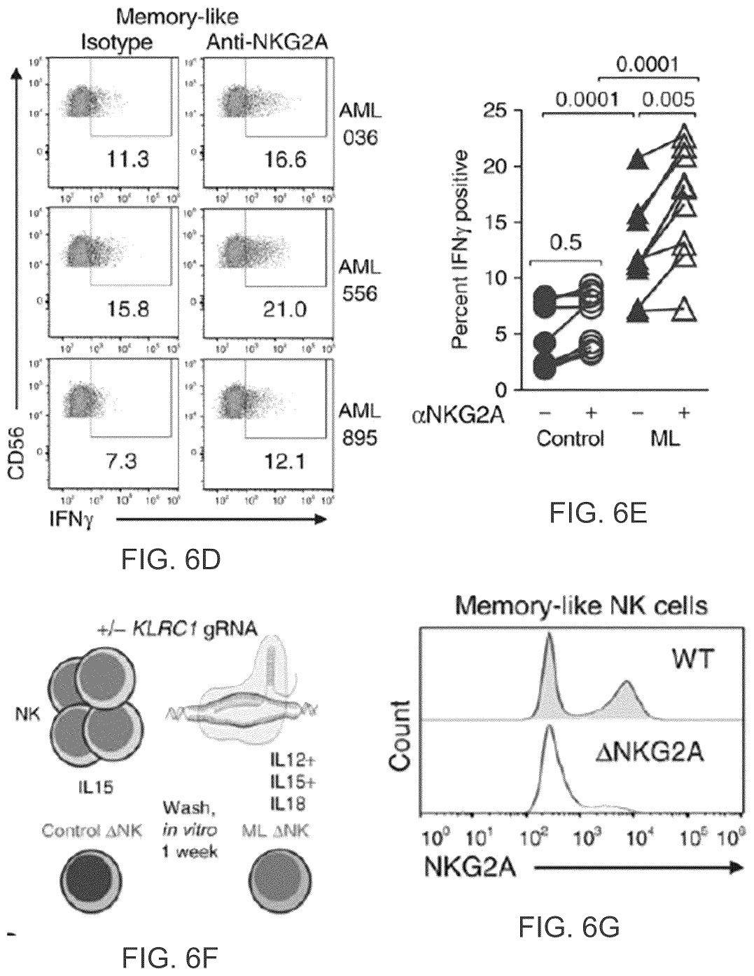

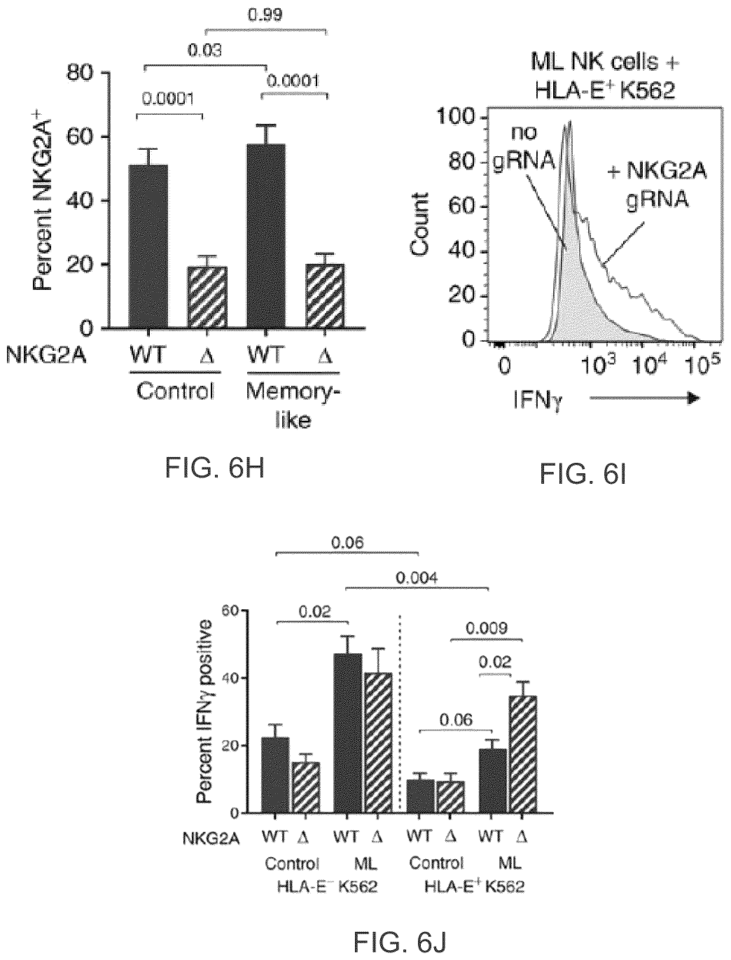

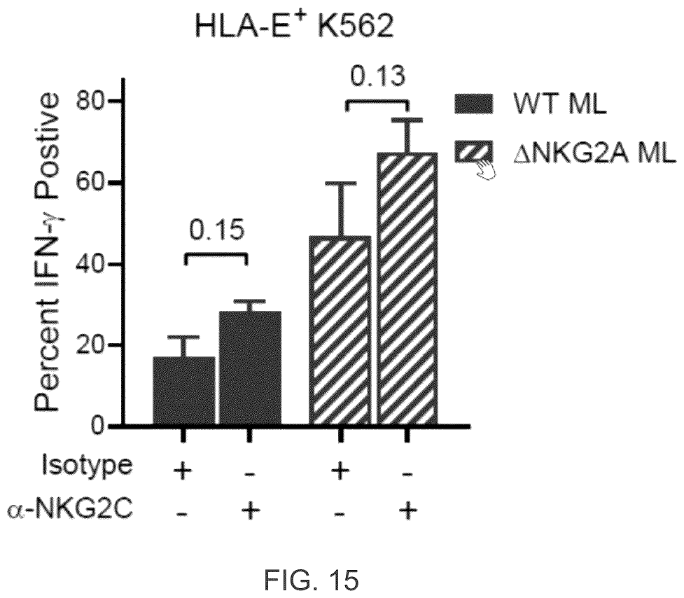

Because NKG2A interactions with HLA-E are inhibitory for ML NK cells, it was hypothesized that abrogating this interaction would restore antileukemia responses (FIG. 6). Indeed, ML NK cell IFNγ production in response to HLA-E⁺ K562 was significantly increased by blocking with anti-NKG2A mAb (FIGS. 6A and B), returning to similar levels as ML NK cells triggered with K562. HLA-E⁺ K562 killing by ML NK cells was also significantly increased in the presence of NKG2A checkpoint blockade compared with isotype mAb (FIG. 6C). NKG2A checkpoint blockade also enhanced ML NK-cell responses, but not control NK-cell responses, to multiple primary AML (FIG. 6D and FIG. 6E). To provide an orthogonal loss-of-function approach, CRISPR/Cas9 was also used to disrupt the NKG2A-encoding gene KLRC1 prior to control or ML NK-cell differentiation (FIG. 6F-FIG. 6J). After electroporation with KLRC1-targeting guide RNA (gRNA) and Cas9 mRNA, cells were rested in vitro for 24 hours and then control (IL15) treated or ML-cytokine (IL12, IL15, and IL18) activated. Cells were allowed to differentiate for 4 to 7 days in IL15, and NKG2A expression was assessed by flow cytometry. Using this approach, NKG2A expression on both control and ML NK cells was significantly reduced (FIGS. 6G and H). WT or ΔNKG2A control or ML NK cells were stimulated with HLA-E⁻ K562 or HLA-E⁺ K562 and IFNγ measured by flow cytometry. Control NK-cell responses were similar in response to K562 with or without HLA-E expression (FIG. 6J), whereas ML NK-cell IFNγ responses were reduced when triggered with HLA-E⁺ K562 (FIG. 6J). NKG2A deletion did not affect the enhanced ML NK-cell responses to K562 (HLA-E⁻), with WT and ΔNKG2A ML NK cells producing similar levels of IFNγ as expected. However, ΔNKG2A ML NK-cell responses were significantly increased compared with WT ML NK-cell responses against HLA-E⁺ K562 (FIG. 6J). To determine whether NKG2C interactions with HLA-E were driving this enhanced response, ΔNKG2A ML NK cells were stimulated with HLA-E⁺ K562 in the presence of α-NKG2C-blocking antibody, or isotype control (FIG. 15). Blocking NKG2C on WT or ΔNKG2A ML NK cells had no impact on IFNγ production in response to HLA-E⁺ K562 leukemia targets. Overall, these data reveal that NKG2A is a critical inhibitor of ML NK-cell responses, but not control NK-cell responses, to AML targets.

CD8⁺ NKG2A+NK Cells Predict Treatment Failure and CD8⁺ NK Cells do not Proliferate in Response to IL12, IL15, and IL18

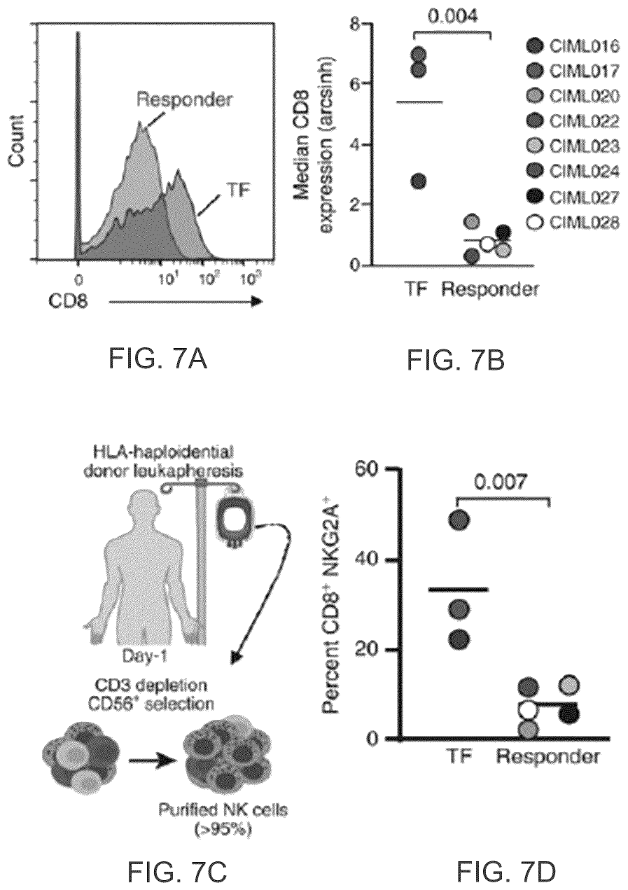

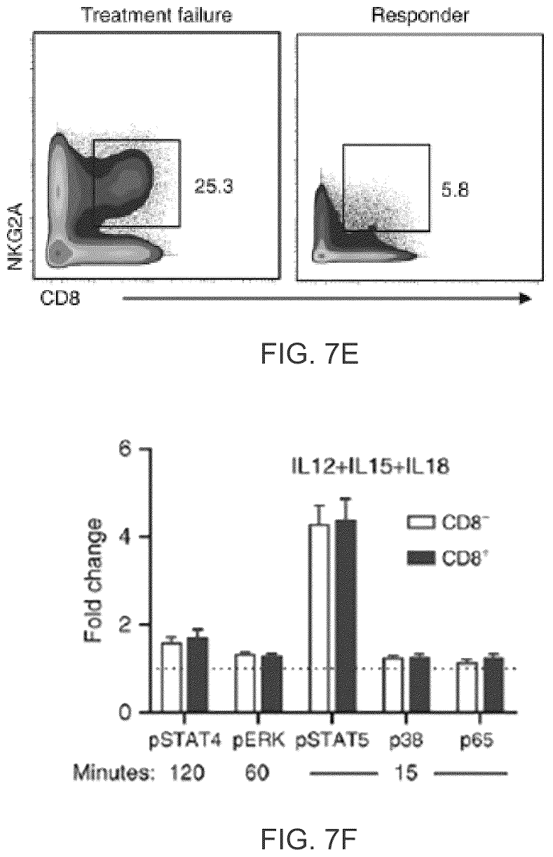

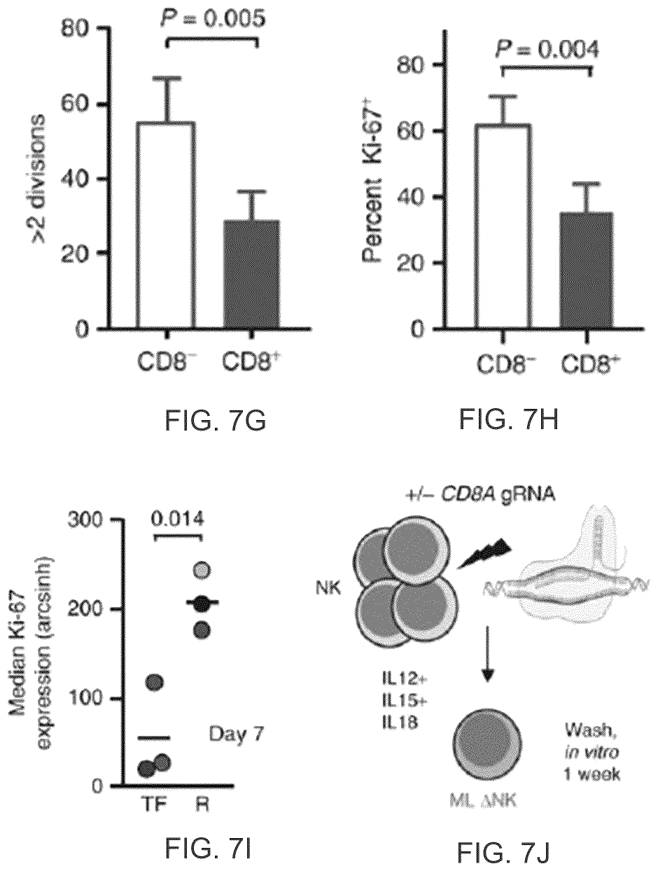

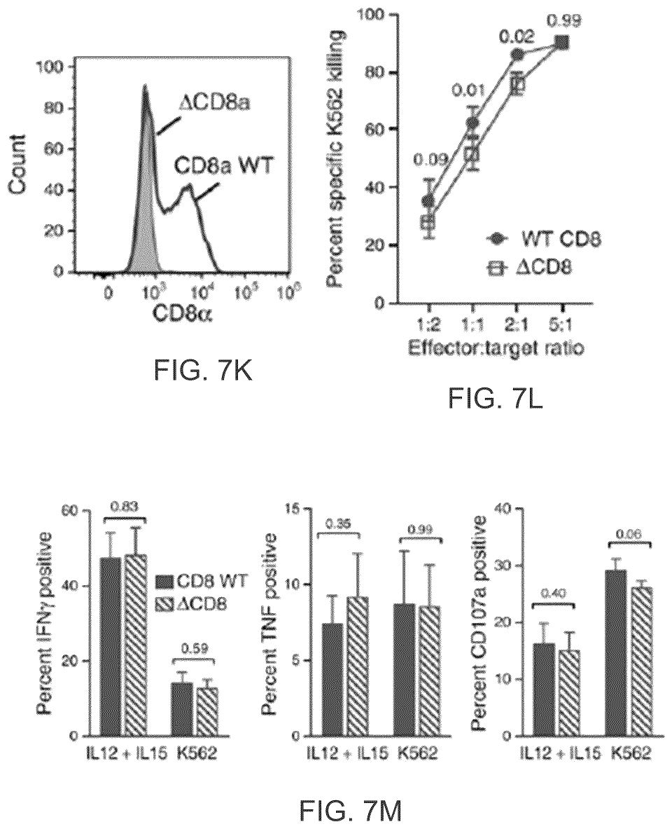

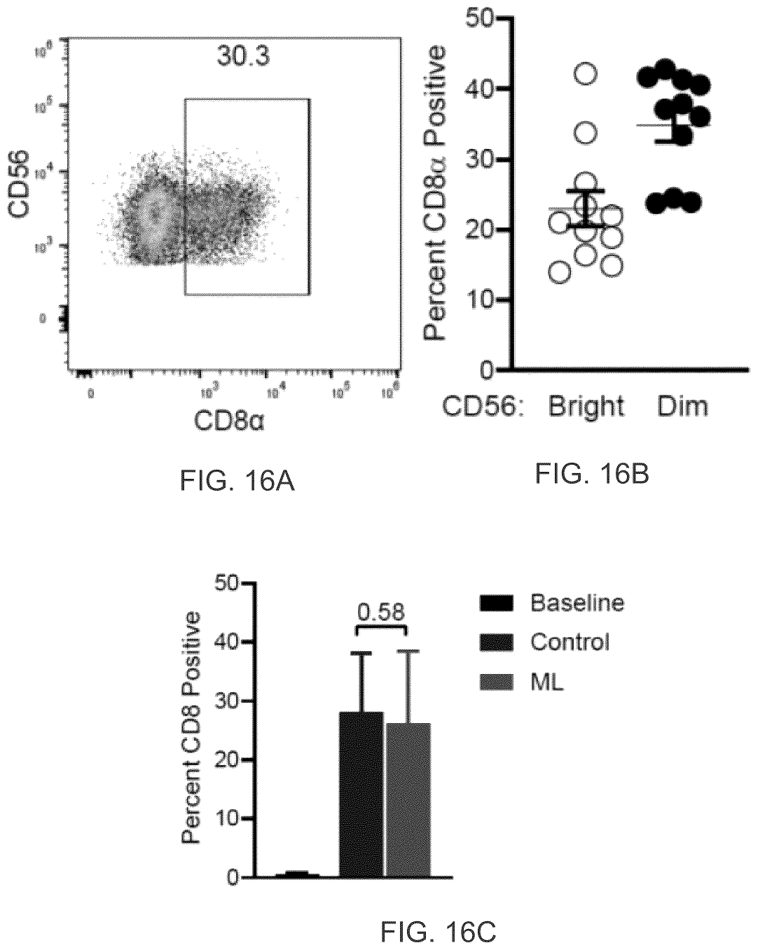

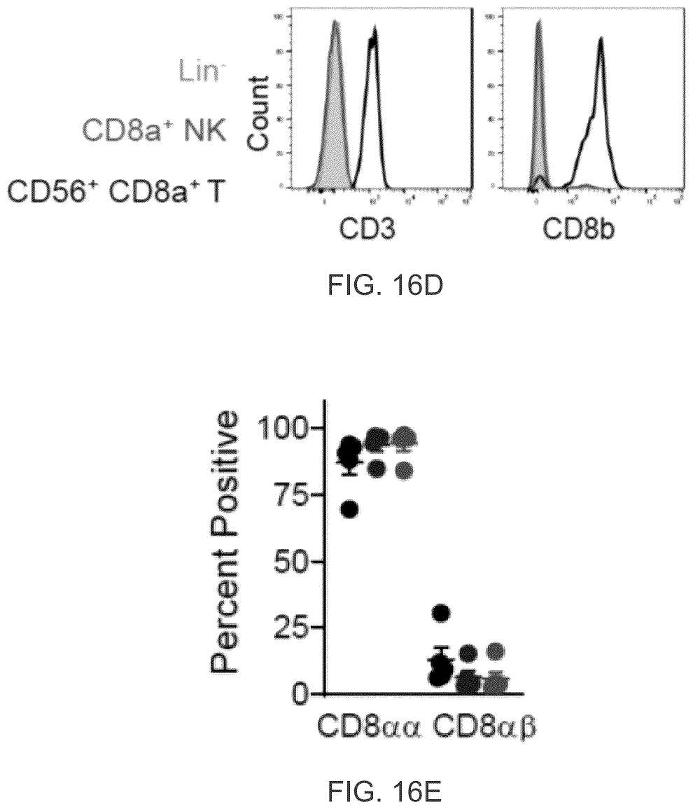

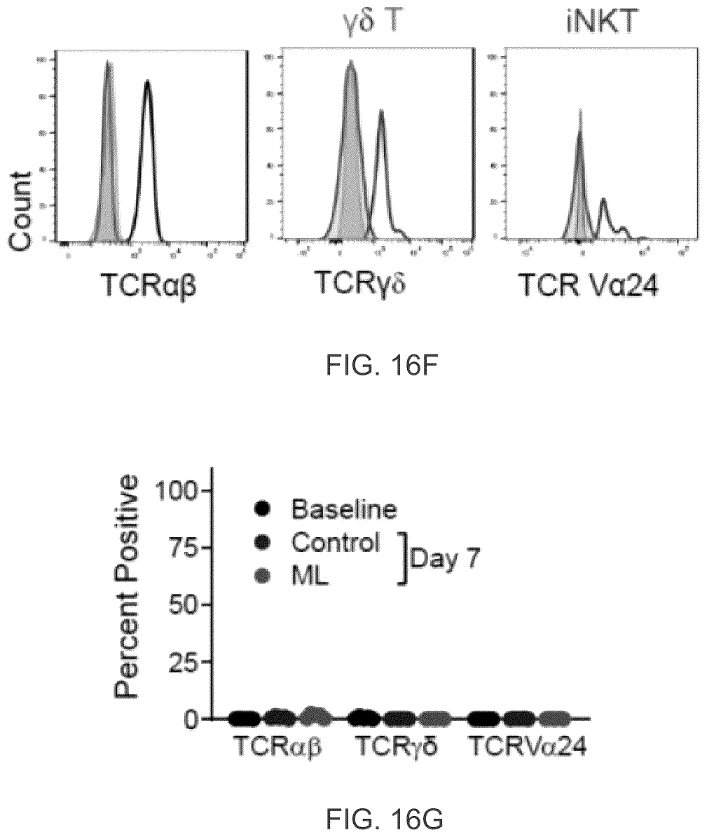

In addition to NKG2A, Citrus unexpectedly identified that CD8a expression on D7 in vivo differentiated ML NK cells was negatively associated with treatment outcomes (SAM, FDR<0.01). No other markers were associated by Citrus with clinical outcomes. Although there was not a significant difference in CD8⁺ ML NK cells in vivo at day 7 (TF: Mean 1322 cells/mL±1,158 cells/mL SD; R: Mean 618.9 cells/mL±701.3 cells/mL SD; unpaired t test P=0.31), median CD8a expression was significantly increased on donor NK cells in the TF patients compared with the responding patients (FIG. 7A and FIG. 7B). Individually, NKG2A or CD8a expression at BL was not associated with clinical responses. To determine whether NKG2A and CD8 coexpression at BL was associated with patient outcomes, the frequency of NKG2A+CD8⁺ NK cells in purified NK-cell products was examined (FIG. 7C-FIG. 7E). The frequency of NKG2A+CD8⁺ NK cells in the product was significantly associated with response to treatment (FIG. 7D and FIG. 7E), with increased frequencies of NKG2A+CD8⁺ NK cells occurring with treatment failure. Consistent with another study (26), CD8α was expressed on approximately 23% of CD56^{bright} and approximately 35% on CD56^{dim} NK cells with a high interindividual variability (FIG. 16A and FIG. 16B). CD8 was not specifically induced in vivo in response to ML NK-cell differentiation (FIG. 9C), but was increased in vitro in both control and ML NK cells. This implicates IL15 signaling in regulating CD8 upregulation in vitro (FIG. 16C). The majority of CD8⁺ NK cells are CD8αα⁺, with a minor subset expressing CD8αβ (FIG. 16D). ML differentiation does not alter these frequencies, relative to control or baseline (FIG. 16E). Finally, CD8⁺ NK cells do not express CD3 or other T-cell receptors (TCR) and represent a subpopulation of NK cells which are distinct from T cells, including iNKT cells (FIG. 16F and FIG. 16G).

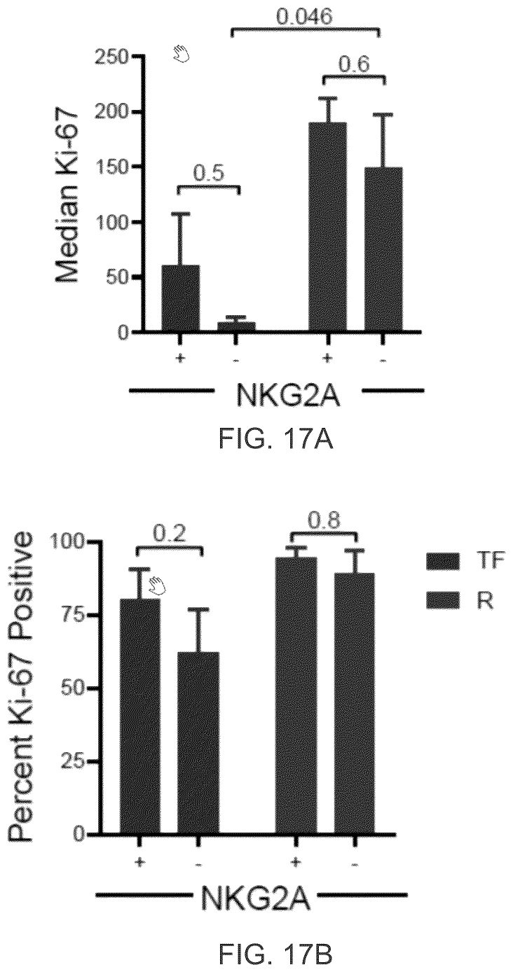

To explain the negative association of CD8 with patient outcomes, it was hypothesized that CD8a⁺ NK cells were not optimally responding to IL12, IL15, and 11_18 activation. To test signaling competency, freshly isolated NK cells were stimulated with IL12, IL15, and IL18 for 0 to 120 minutes, and phosphorylation of proximal cytokine signaling molecules STAT4, ERK, STAT5, p38, and p65 was measured (27, 28). For both CD8a⁺ and CD8a⁻ NK cells, similar phosphorylation was observed relative to the unstimulated condition (P>0.05; one sample t test, test value=1; FIG. 7F). No differences in cytokine receptor signaling were observed between CD8⁺ and CD8⁻ NK cells (FIG. 7F). Next, the ability of CD8a⁺ and CD8α⁻ NK cells to proliferate in response to ML-cytokine activation was compared. Sorted CD8α⁺ and CD8α⁻ NK cells were cell trace violet (CTV)-labeled, activated with IL12, IL15, and IL18, washed after 16 hours, and allowed to differentiate. Proliferation was assessed after 6 days. CD8α⁺ NK cells divided less compared with CD8⁻ NK cells, and expression of Ki-67 was reduced, both indicating significantly inferior proliferation (FIGS. 7G and H). It was hypothesized that the larger number of CD8α⁺ donor NK cells infused into TF patients were not expanding to the same extent as the predominantly CD8α⁻ donor NK cells in responding patients. Consistent with this, the amount of Ki-67 in donor NK cells was significantly associated with treatment outcomes (FIG. 7I). However, median Ki-67 expression between NKG2A+ and NKG2A⁻ ML NK cells in TF and responders was not significantly different (FIG. 17A and FIG. 17B). Patients with donor ML NK cells with lower Ki-67 expression failed treatment. However, in this small sample size, total donor NK-cell numbers in the PB at a single time point (7 days) measured post-infusion did not directly correlate with response (FIG. 8D). Although these data provide insight into the mechanisms underlying treatment failure, they include a single time point, and further studies are needed. These in vivo data are consistent with the in vitro observations that CD8α⁺ ML NK cells do not proliferate as strongly as CD8α⁻ ML NK cells, and may explain the inferior clinical responses.

To evaluate the cell-intrinsic role for CD8α on ML NK-cell functionality, ΔCD8a ML NK cells were compared with WT ML NK cells in in vitro functional assays (FIG. 7J-FIG. 7M). Using this approach, CD8α expression was reduced on ΔCD8a ML NK cells compared with WT ML NK cells (FIG. 7K). K562 target killing by ΔCD8a ML NK cells was slightly, yet significantly, decreased compared with WT ML NK cells (FIG. 7L). Furthermore, in response to cytokines and tumor targets, IFNγ, TNF, and CD107a were similar between ΔCD8a ML NK cells compared with WT ML NK cells (FIG. 7M). These data suggest that CD8α does not impair ML NK-cell responses to prototypical stimuli, but further studies are warranted.

Discussion

Here multidimensional immune correlative phenotyping by mass cytometry was used to identify the in vivo differentiated human ML NK-cell phenotype, which was distinct from cytokine-activated and conventional NK cells. ML NK cells were safe, expanded in vivo, and induced IWG responses in 67% (47% CR/CRi) of evaluable patients. It was demonstrated that NKG2A is transcriptionally regulated in ML NK cells and represents a critical induced checkpoint for cytokine-induced ML NK-cell responses, associating with treatment failure in patients with AML treated with donor ML NK cells. Although little is known about the role of CD8α on NK cells, a new association with CD8a⁺ NK cellular therapy and inferior patient outcomes was identified here, likely due to their inability to robustly proliferate in response to combined cytokine activation.

NKG2A is a C-type lectin inhibitory receptor that heterodimerizes with CD94 and recognizes the nonclassic class I-MHC HLA-E, resulting in ITIM-mediated NK-cell inhibition (22). NKG2A expression on baseline donor NK cells was not associated with clinical outcomes. Furthermore, the importance of NKG2A for conventional (naïve) or control (low-dose IL15 supported) NK-cell response to HLA-E⁺ tumor targets was not observed, suggesting that NKG2A is a minor inhibitory receptor on conventional NK cells. Here it is demonstrated that NKG2A is an inducible checkpoint molecule on cytokine-induced ML NK cells and is a critical inhibitor of ML NK-cell responses against HLA-E⁺ tumor targets. NKG2A can be transcriptionally induced during ML differentiation. However, both enhanced proliferation of NKG2A+NK cells and de novo NKG2A upregulation are likely operative in regulating overall NKG2A expression during ML NK-cell differentiation. The lab's previous report showed that ML NK cells are not inhibited through the regular rules of inhibitory KIR to KIR-ligand interactions (5). Data presented here indicate that ML NK cells are instead primarily inhibited through NKG2A, and further studies examining the role for NKG2A and immune tolerance in the setting of cytokine activation and inflammation are warranted. Moreover, the fraction of NKG2A+NK cells does not appear as important as the per-cell NKG2A expression, because nearly all donor ML NK cells expressed NKG2A, but only those donors with supraphysiologic expression were associated with treatment failure. However, HLA-E expression was overall increased in the treatment-failure tumor microenvironment, suggesting both NKG2A supraexpression and increased HLA-E contribute to resistance to ML NK cellular therapy. Although future work will elucidate the mechanisms of supraphysiologic expression by some donors, here it is shown that NKG2A is transcriptionally induced after IL12, IL15, and IL18 activation and is associated with a concomitant increase in GATA3, a known regulator of NKG2A, as well as EOMES, which is important for establishing a central memory phenotype in CD8⁺ T cells (25). The interplay between these two transcription factors and how they establish ML NK-cell differentiation program is unclear, but this is an active area for further investigation. Translationally, blockade of NKG2A or gene editing of KLRC1 represent exciting potential strategies to improve on ML NK cellular therapy. Preclinical studies utilizing these approaches are ongoing and critical for establishing proof-of-principle needed to move this strategy into the clinic. Indeed, recent reports have demonstrated that combination anti-NKG2A and anti-PD-L1 mAb controlled tumor growth in murine models of B- and T-cell lymphoma, as well as established the safety of anti-NKG2A mAb for patients with squamous cell carcinoma of the head and neck (29), supporting the feasibility of translating these findings to the clinic.

There are limited reports of the role of CD8 on human NK cells, which is normally expressed as a CD8α homodimer, leaving CD8 receptor biology unclear in this context. Previous reports indicate that CD8⁺ NK cells have enhanced cytotoxicity and undergo reduced activation induced-apoptosis (26, 30). In addition, the presence of CD8⁺ NK cells has been associated with slower HIV-1 progression in chronically infected individuals (31). CD8α expression on NK cells was reported to contribute to KIR3DL1 signaling (32). Stronger CD8 interactions with MHC-I were hypothesized to improve licensing by enhancing KIR-KIR-ligand interactions, which is one possible mechanism for increasing NK-cell functionality (32). However, this study implies a negative role for CD8a⁺ NK cells in adoptive NK-cell immunotherapy. Potentially explaining this conundrum, it is shown here that CD8a⁺ NK cells have reduced proliferative capacity compared with CD8α⁻ NK cells. There are some studies examining the role of CD8αα in limiting T-cell responses (33). CD8αβ is a well-characterized coreceptor for TCR interactions with MHC-I, and enhances TCR signaling (34). However, studies have implicated that CD8αα inhibits T-cell responses and that CD8αα may act as an inhibitory molecule in nonclassic T-cell subsets (33, 35). Although CD8α⁺ NK cells exhibit reduced proliferation, it remains unclear if CD8α is a marker of a differentiated, terminal phenotype with limited replicative capacity, or if CD8 is directly inhibiting proliferation in vivo. The negative association of CD8 expression with clinical outcomes following NK-cell immunotherapy identifies the importance of understanding its role on NK cells, as well as the potentially distinct biology of CD8α⁺ NK cells from CD8α⁻ NK cells.

Here is reported the first high-dimensional characterization of in vivo differentiated ML NK cells in the context of the final phase I clinical data, demonstrating the safety and efficacy of ML NK cells to treat patients with rel/ref AML. ML NK cells are well tolerated and did not cause GVHD, CRS, or (CANS, nor grade 3 adverse events related to ML NK-cell infusion. The observed CR/CRi rate of 47% is remarkable for a population of older adults with rel/ref AML, and is consistent with the lab's initial report. Although the duration of response was relatively short (2-6 months) for most patients, one patient, who became HCT-eligible, had a durable response that persisted after allogeneic HCT. This strategy as a “bridge to HCT” is being tested in the lab's phase II cohort for rel/ref AML (NCT01898793). Based upon their ability to ignore inhibitory KIR ligation, the effectiveness of ML NK cells against solid tumors is also being evaluated (36), and expanding their repertoire against NK-resistant tumors using bispecific triggering (37) as well as chimeric antigen receptor engineering (38). With these extensive immune correlative studies, here NKG2A has been identified as a targetable checkpoint that could be combined with ML NK-cell adoptive therapy in future trials. Moreover, a new strategy for donor NK-cell donor selection based on NKG2A+CD8⁺ NK cell frequency was discovered. Multiple clinical trials at Washington University utilizing ML NK-cell adoptive immunotherapy, including as a bridge to HCT (NCT01898793), as augmentation of MHC-haploidentical HCT with same-donor ML NK cells (NCT02782546), and as therapy for relapse after allogeneic HCT (NCT03068819, refs. 39, 40) have been reported. As evidenced by this study, multidimensional immune correlates will be performed to understand if NKG2A and CD8 can predict patient outcomes in other ML NK-cell clinical contexts. Thus, this study highlights the importance of multidimensional immune monitoring to identify mechanisms of response and resistance following NK-cell therapy.

Methods

Study Design

Patients treated on an open-label, nonrandomized, phase I dose-escalation trial (NCT01898793) are included in this study. Prior to any study-related testing or treatment, written informed consent was obtained from all patients under a Washington University School of Medicine Institutional Review Board (IRB)— approved clinical protocol, and all studies were conducted in accordance with the Declaration of Helsinki. The initial escalation was previously reported (5). Briefly, patients were treated with fludarabine/cyclophosphamide between days −7 and −2 for immunosuppression, followed on day 0 by allogeneic donor IL12, IL15, and IL18 activated NK cells. Patients in dose level 3 received the maximum NK cells that could be generated (capped at 1×10⁷ cells/kg). After donor NK-cell transfer, rhIL2 was administered subcutaneously every other day for a total of 6 doses. Donor NK cells were purified from a nonmobilized apheresis product using CD3 depletion followed by CD56⁺ selection (CliniMACS device). Purified NK cells were activated with IL12 (10 ng/mL), IL15 (50 ng/mL), and IL18 (50 ng/mL) for 12 hours under current GMP conditions.

Samples were obtained from the PB (day 7, 8, and 14 after infusion) and BM (screening, day 8 and 14 after infusion). Clinical responses were defined by the revised IWG criteria for AML (15). All patients provided written informed consent before participating and were treated on a Washington University IRB-approved clinical trial (Human Research Protection Office #201401085).

Reagents and Cell Lines

Anti-human mAbs were used for flow and mass cytometry (Supplementary Methods, TABLE 4). Endotoxin-free, recombinant human (rh) IL12 (BioLegend), IL15 (Miltenyi Biotec), and IL18 (InVivo Gen) were used in these studies. K562 cells (ATCC, CCL-243) were obtained in 2008, viably cryopreserved, and maintained for <2 months at a time in continuous culture according to ATCC specifications. K562 cells were authenticated in 2015 using single-nucleotide polymorphism analysis and were found to be exactly matched to the K562 cells from the Japanese Collection of Research Bioresources, German Collection of Microorganisms and Cell Cultures (DSMZ), and ATCC databases (Genetic Resources Core Facility at Johns Hopkins University). HLA-E⁺ K562 were a gift from Dr. Deepta Bhattacharya (Washington University School of Medicine). These cells were generated using the AAVS1-EF1a donor plasmid containing the coding sequence for human HLA-E. The K562 cells were electroporated using a Bio-Rad Gene Pulse electroporation system. HLA-E⁺ cells were sorted to >98% purity.

NK-Cell Purification and Cell Culture

Normal donor PBMCs were obtained from anonymous healthy platelet donors. NK cells were purified using RosetteSep (StemCell Technologies; routinely >95% CD56⁺ CD3⁻). ML and control NK cells were generated as described previously (5). Cells were maintained in 1 ng/mL IL15, with media changes every 2 to 3 days. For proliferation assays, cells were labeled with 2.5 μmol/L CTV (Life Technologies) for 15 minutes at 37° C.

Patient Samples

Patients with newly diagnosed AML provided written informed consent under the Washington University IRB-approved protocol (Human Research Protection Office #2010-11766) and were the source of primary AML blasts for in vitro stimulation experiments. Patient PBMCs and BMMCs were isolated by Ficoll-Paque PLUS (GE Health) centrifugation and immediately used in experiments. For assessing HLA-E expression in patient BM, viably frozen cells were thawed and stained immediately using mass cytometry.

Functional Assays to Assess Cytokine Production

For patient stimulation assays, PBMCs or BM cells were stimulated in a standard functional assay (5). Cells were stimulated with K562 leukemia targets (5:1 effector-to-target ratio). Functionality was measured using mass cytometry as described previously (5). For each patient sample, a normal donor sample was thawed and stimulated with K562 and used as a control for the functional assay. For in vitro differentiated NK-cell functional assays, control and ML NK cells were harvested after a rest period of 5 to 7 days to allow memory-like NK-cell differentiation to occur. Cells were incubated with K562±HLA-E or freshly thawed primary AML blasts. All cytokine secretion assays were performed for 6 hours in the presence of GolgiPlug/GolgiStop (BD Biosciences) for the final 5 hours. Anti-CD107a antibodies were included in the well at the beginning of the assay to measure degranulation. For antibody blocking experiments, 10 μg/mL anti-NKG2A (Z199), anti-NKG2C (134522), or isotype control (IgG) were added directly to the wells at the beginning of the assay.

Flow-Based Killing Assay

Flow-based killing assays were performed by coincubating ML or control NK cells with carboxyfluorescein succinimidyl ester (CFSE)-labeled K562±HLA-E for 4 hours and assaying 7-aminoactinomycin D (7AAD) uptake as described previously (13).

qRT-PCR

Cells were resuspended in TRIzol and RNA extracted using Zymo Directzol RNA microPrep according to the manufacturers directions. cDNA was generated using Life Technologies High Capacity cDNA Reverse Transcription Kit (4368814) according to the manufacturer's instructions. Real-time qPCR was performed using ABI Master Mix with TaqMan Gene Expression Assay, Hs00970273_g1 KLRC1, Hs00935338_m1 BACH2, and Hs01060665_g1 ACTB, according to the manufacturer's instructions. Samples were analyzed on StepOnePlus Real-Time PCR system (Applied Biosystems). Relative quantification was determined by ΔΔ threshold cycle method, by normalizing KLRC1 or BACH2 to ACTB (β-actin).

RNA Sequencing and GSEA

Cells were stored in TRIzol at −80° C. until RNA isolation using the Direct-zol RNA MicroPrep Kit (Zymo Research). NextGen RNA sequencing was performed using an Illumine HiSeq 2500 sequencer. RNA-sequencing reads were then aligned to the Ensembl release 76 primary assembly with STAR version 2.5.1a. Gene counts were derived from the number of uniquely aligned unambiguous reads by Subread:featureCount version 1.4.6-p5. Analysis of sequencing data was performed using Phantasus, a browser-based gene expression analysis software. Genes were log₂ normalized and filtered to remove duplicate reads and low-expressed genes. Differential expression analysis was performed on the top 12,000 expressed genes using the LIMMA package to analyze differences between conditions. GSEA was performed using the Harmonizome database of GATA3 target genes (41).

Flow Cytometric Analysis and Sorting

Cell staining was performed as described previously (5), and data were acquired on a Gallios flow cytometer (Beckman Coulter) and analyzed using FlowJo (Tree Star) software. Dead cells were stained using Zombie reagent (BioLegend) according to the manufacturer's instructions, except for phospho-flow. eBio Fix/Perm was used for all intracellular staining except phospho-flow. For phospho-flow, cells were stimulated with IL12, IL15, and IL18 for 0, 15, 60, and 120 minutes. Cells were fixed in 1% prewarmed formalin and permeabilized using 100% ice-cold methanol. Cells were washed three times and stained overnight, as described previously (42). CD8⁺ and CD8⁻ NK cells were sorted to >99% purity using FACSAria II Cell Sorter (BD Biosciences) or purified using Automacs column (Miltenyi Biotec). CD56^{dim} CD16⁺ NKG2A+ and NKG2A⁻ NK cells were sorted using FACSAria II cell sorter (BD Biosciences).

Mass Cytometry

Mass cytometry was performed on freshly isolated patient PB or BM cells as described previously (5, 43), or on thawed BM samples. Cells were stained for live/dead using cisplatin, and surface staining performed at 4° C. for 15 minutes. Cells were washed, and fixed with eBio Fix/Perm overnight at 4° C. Cells were stained using intracellular antibodies at 4° C. for 15 minutes. Cells were washed and resuspended in PBS containing 1% paraformaldehyde and stored until all samples were stained. Once all samples were stained, they were washed and barcoded according to the manufacturer's instructions. Data were collected on a Helios mass cytometer (Fluidigm) and analyzed using Cytobank (44). Data were analyzed using previously described methods (45). KIR diversity (KIR2DL1, KIR2DL2/2DL3, KIR3DL1, KIR2DS4, KIR2DL5) was assessed at baseline and after in vivo differentiation as described previously (5). For each patient sample, a normal donor PBMC sample was thawed and stained, providing a staining control at day 0, day 7, and day 8 of patient sample staining. These were used for quality control. Staining for each marker was confirmed to be consistent across the days on which the samples were stained using the same master mix. Comparisons were also made (Student t test or Mann-Whitney) between matched normal donors stained on DO and D7. For all markers, there were not significant differences in median expression between normal donors thawed and stained on DO or D7. These control samples served to increase the confidence that changes observed in the patient samples represented biologically relevant changes and did not reflect technical issues that could arise from these assays. Citrus was performed assessing median with default settings, using the same clustering channels as the viSNE (FIG. 1; TABLE 4), with the addition of CD8. To define the in vivo ML differentiated phenotype (FIG. 1) and the lymphocyte subsets all patients with mass cytometry data available were used, including CIML026 who expired prior to day 28, post-infusion, and was thus not evaluable for response (male, aged 77, diagnosed with AML, treated at dose level 3, 2 prior therapies, 7% blasts prior to treatment). CIML025 was evaluable but was treated at a dose level 2 and was excluded from the Citrus analyses (FIG. 5 and FIG. 7). Phenotypic intracellular markers for CI ML028 were not assessed. For CIML020, an inappropriate amount of Ki-67 antibody was used, making the median expression value an outlier [ROUT (Q=1%)]; however, percent positive could still be reliably assessed. For functional assays, CD107a was omitted from the functional assay for CIML026. For patient CIML027, due to limiting cells in the PB, BM donor NK cells were used to assess licensing. CIML028 had only had cells available for a functional assay at D14.

CRISPR/Cas9 Gene Editing

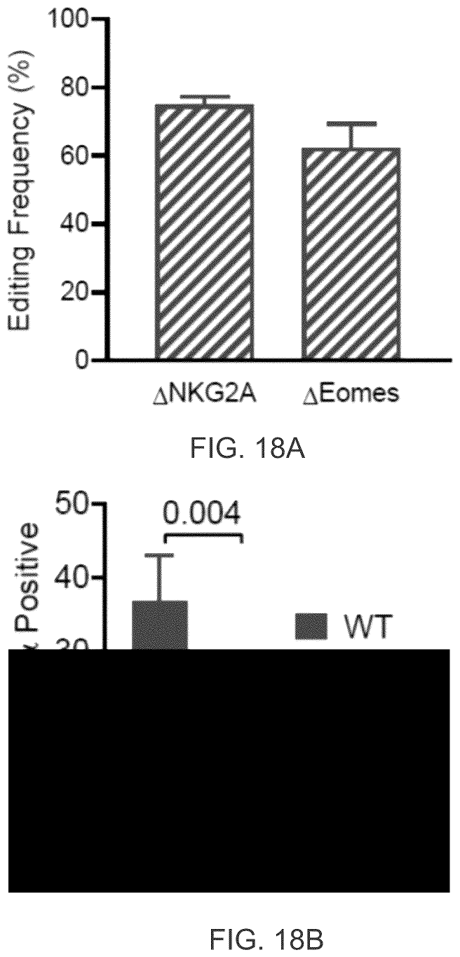

NK cells were purified from normal donors and rested overnight in 1 ng/mL IL15. Cells were washed with PBS, two times to remove serum, and resuspended in MaxCyte EP buffer plus CAS9 mRNA (Trilink; ref. 46). Next, gRNA [NKG2A: AACAACUAUCGUUAACCACAG (SEQ ID NO: 5) (Trilink, Synthego); EOMES: AACCAGUAUUAGGAGACUCU (SEQ ID NO: 6) (IDT); or CD8A: GACUUCCGCCGAGAGAACGA (SEQ ID NO: 7) (IDT); 2×10⁸ cells/mL] or no gRNA (control) was added to the cells, which were then electroporated in a Maxcyte GT using the WUSTL-2 setting in an OC-100 processing assembly. Cells were removed from the OC-100 and incubated for 10 minutes at 37° C. Prewarmed media containing 3 ng/mL IL15 was added and cells rested for 24 hours. Cells were then control treated (3 ng/mL IL15) or cytokine activated (IL12/15/18) for 16 to 18 hours, as described previously (10). Cells were washed three times and maintained in complete RPMI supplemented with 10% Human AB serum and 3 ng/mL IL15. Media changes were performed every 2 to 3 days. Gene-editing efficiency was determined as described previously (FIG. 18A and FIG. 1813, ref. 47).

Statistical Analysis

Before statistical analyses, all data were tested for normal distribution (Shapiro-Wilk). If data were not normally distributed, the appropriate nonparametric tests were used (GraphPad Prism v8), with all statistical comparisons indicated in the figure legends. Uncertainty is represented in figures as SEM, except where indicated. All comparisons used a two-sided a of 0.05 for significance testing.

Supplementary Materials

Study Results

Eighteen patients received ML NK infusion but 3 were unevaluable due to insufficient cell dose (n=1) or early death (n=2). Among the 15 evaluable patients, 14 had AML and 1 MDS (clinical summaries in TABLE 1). Median age was 72 years (range, 43-83), and median number of prior therapies was 2 (range, 1-7). Among the toxicities attributed to ML NK cells, none occurred in more than one patient, and all were grade 1-2 (TABLE 2 and TABLE 3). There were no dose limiting toxicities, and no deaths were attributed to ML NK cells. No GVHD or CRS was observed. Among the 15 evaluable patients, 7 achieved CR (n=3) or CRi (n=4), and 3 had a best response of morphologic leukemia free state at day 14 by the IWG response criteria (15), for an overall response rate of 67% and a CR/CRi rate of 47%. Median leukemia-free survival among responding patients was 84 days, with one patient in ongoing remission after allogeneic stem cell transplant.

Two AML patients were retreated with lymphodepleting chemotherapy and infusion of ML-NK cells prepared from their original donors. The first was a 77 year-old man who achieved a CR lasting 126 days after his first course of study treatment. He then received a second infusion of ML-NK cells 57 days after disease progression. He had no evidence of leukemia on day 14 but died of sepsis on day 19. The second was a 71 year-old female who achieved a CR lasting 73 days after the first infusion of ML-NK cells. She received a second infusion of ML-NK cells 84 days after disease progression and achieved a second CR lasting 121 days, until her death from pneumonia.

Materials and Methods

Flow Cytometry Antibodies

Cells were stained for viability using zombie NIR (Biolegend), according to manufacturer's instructions and then surface antibody staining was performed for 15 minutes at 4° C. in FACS buffer (PBS, 2% FBS, 1 mM EDTA). Cells were washed twice and fixed using eBiosciences Fix/Perm, according to manufacturer's instructions. Permeabilized cells were stained for intracellular markers for 30 minutes at 4° C. in 1× permeabilization buffer. Cells were washed and assessed. The following antibodies BD antibodies were used: CD8 (SKI), CD16 (3G8), Ki67 (B56), phospho (p)-STAT4 (38/p-Stat4), p-STAT5 (47/Stat5, pY694), p-ERK (pT202/pY204), p38 (pT290/pY182), p65 (pS529), and HLA-A2 (667.2), TCR-ab (WT131), Blimp-1 (6D3). The following Biolegend antibodies were used: T-bet (4610), CD107a (H4A3), IFN-γ (B27), GATA3 (16E10A23), HLA-A2 (BB7.2) and HLA-E (3D12), TCR Va24-Ja18 (6B11), TCR gd (B1), TCF7 (7F11A10). The following eBiosciences antibodies were used: E4BP4 (MABA223), EOMES (WD1928), and HLA-A3 (GAP.A3). The following Beckman Coulter antibodies were used: CD45 (A96416), CD3 (UCHT1), and NKG2A (Z199). Runx3 (CBFA3) was purchased from R&D. The following Miltenyi antibodies were used: HLA-Bw6 (REA143), HLA-A9 (REA127), HLA-A2 (REA517), HLA-A2/A28 (REA142), HLA-Bw4 (REA274). Runx2 (DIL7F) was purchased from Cell Signaling.

CRISPR/Cas9 Gene Editing Efficiency

DNA was isolated from NK cells electroporated with Cas9 mRNA (Trilink) and respective sgRNA (Trilink, IDT, and Synthego) from 5-6 donors using Qiagen gentra puregene kit, according to manufacturer's instructions. CRISPR editing efficiency was determined by Next Generation Sequencing of the region around the respective sgRNA targeting site. Amplicons were prepared using the primers F_5′AGAAGCTCATTGTTGGGATCCTG3′ (SEQ ID NO: 1) and R_5′ACAATGAGAACTCTATTCCCTGAAA3′ (SEQ ID NO: 2) for NKG2A (KLRC1); and F_5′AGCTAAGAGACATCCCTCCG3′ (SEQ ID NO: 3), R_5′ CTCTGTCACTCTACCTGGGTGR3′ (SEQ ID NO: 4) for Eomes. Sequencing data were analyzed with CRISPResso2 (48). CRISPR editing efficiency was calculated as 100-Percent of WT allele reads.

Data and Materials Availability

The RNA-sequencing data are accessible within Gene Expression Omnibus (GEO) under accession code GSE154694.

### REFERENCES

1. Vivier E, Tomasello E, Baratin M, Walzer T, Ugolini S. Functions of natural killer cells. Nat Immunol 2008; 9:503-10.
2. Lanier L L. NK cell recognition. Ann Rev Immunol 2005; 23:225-74.
3. Estey E, DOhner H. Acute myeloid leukaemia. Lancet 2006; 368:1894-907.
4. Miller J S, Soignier Y, Panoskaltsis-Mortari A, McNearney S A, Yun G H, Fautsch S K, et al. Successful adoptive transfer and in vivo expansion of human haploidentical NK cells in patients with cancer. Blood 2005; 105:3051-7.
5. Romee R, Rosario M, Berrien-Elliott M M, Wagner J A, Jewell B A, Schappe T, et al. Cytokine-induced memory-like natural killer cells exhibit enhanced responses against myeloid leukemia. Sci Transl Med 2016; 8:357ra123.
6. Shimasaki N, Jain A, Campana D. NK cells for cancer immunotherapy. Nat Rev Drug Discov 2020; 19:200-18.
7. Berrien-Elliott M M, Romee R, Fehniger T A. Improving natural killer cell cancer immunotherapy. Curr Opin Organ Transplant 2015; 20:671-80.
8. Hirayama A V, Turtle C J. Toxicities of CD19 CAR-T cell immunotherapy. Am J Hematol 2019; 94:S42-9.
9. Simonetta F, Pradier A, Bosshard C, Masouridi-Levrat S, Chalandon Y, Roosnek E. NK cell functional impairment after allogeneic hematopoietic stem cell transplantation is associated with reduced levels of T-bet and eomesodermin. J Immunol 2015; 195:4712-20.
10. Romee R, Schneider S E, Leong J W, Chase J M, Keppel C R, Sullivan R P, et al. Cytokine activation induces human memory-like NK cells. Blood 2012; 120:4751-60.
11. Ni J, Miller M, Stojanovic A, Garbi N, Cerwenka A. Sustained effector function of IL-12/15/18-preactivated NK cells against established tumors. J Exp Med 2012; 209:2351-65.
12. Cooper M A, Elliott J M, Keyel P A, Yang L, Carrero J A, Yokoyama W M. Cytokine-induced memory-like natural killer cells. Proc Natl Acad Sci USA 2009; 106:1915-9.
13. Leong J W, Chase J M, Romee R, Schneider S E, Sullivan R P, Cooper M A, et al. Preactivation with IL-12, IL-15, and IL-18 induces CD25 and a functional high-affinity IL-2 receptor on human cytokine-induced memory-like natural killer cells. Biol Blood Marrow Transplant 2014; 20:463-73.
14. Wagner J A, Berrien-Elliott M M, Rosario M, Leong J W, Jewell B A, Schappe T, et al. Cytokine-induced memory-like differentiation enhances unlicensed NK cell anti-leukemia and FcγRIIIa-triggered responses. Biol Blood Marrow Transplant 2016; 23:398-404.
15. Cheson B D, Bennett J M, Kopecky K J, Buchner T, Willman C L, Estey E H, et al. Revised recommendations of the international working group for diagnosis, standardization of response criteria, treatment outcomes, and reporting standards for therapeutic trials in acute myeloid leukemia. J Clin Oncol 2003; 21:4642-9.
16. Van Gassen S, Callebaut B, Van Heiden M J, Lambrecht B N, Demeester P, Dhaene T, et al. FIowSOM: Using self-organizing maps for visualization and interpretation of cytometry data. Cytom Part A 2015; 87:636-45.
17. Bachanova V, Burns L J, McKenna D H, Curtsinger J, Panoskaltsis-Mortari A, Lindgren B R, et al. Allogeneic natural killer cells for refractory lymphoma. Cancer Immunol Immunother 2010; 59:1739-44.
18. Gumá M, Angulo A, Vilches C, Gomez-Lozano N, Malats N, Lopez-Botet M, et al. Imprint of human cytomegalovirus infection on the NK cell receptor repertoire. Blood 2004; 104:3664-71.
19. Foley B, Cooley S, Verneris M R, Pitt M, Curtsinger J, Luo X, et al. Cytomegalovirus reactivation after allogeneic transplantation promotes a lasting increase in educated NKG2C+ natural killer cells with potent function. Blood 2012; 119:612-26.
20. Horowitz A, Strauss-Albee D M, Leipold M, Kubo J, Nemat-Gorgani N, Dogan O C, et al. Genetic and environmental determinants of human NK cell diversity revealed by mass cytometry. Sci Transl Med 2013; 5:208ra145.
21. Bruggner R V, Bodenmiller B, Dill D L, Tibshirani R J, Nolan G P. Automated identification of stratifying signatures in cellular subpopulations. Proc Natl Acad Sci USA 2014,111:E2770-7.
22. Lee N, Llano M, Carretero M, Ishitani A, Navarro F, Lopez-Botet M, et al. HLA-E is a major ligand for the natural killer inhibitory receptor CD94/NKG2A. Proc Natl Acad Sci USA 1998; 95:5199-204.
23. Björkström NK, Riese P, Heuts F, Andersson S, Fauriat C, Ivarsson M A, et al. Expression patterns of NKG2A, KIR, and CD57 define a process of CD56^{dim} NK-cell differentiation uncoupled from NK-cell education. Blood 2010; 116:3853-64.
24. Marusina Al, Kim D-K, Lieto L D, Borrego F, Coligan J E. GATA-3 is an important transcription factor for regulating human NKG2A gene expression. J Immunol 2005; 174:2152-9.
25. Banerjee A, Gordon S M, Intlekofer A M, Paley M A, Mooney E C, Lindsten T, et al. Cutting edge: The transcription factor eomesodermin enables CD8+ T cells to compete for the memory cell niche. J Immunol 2010; 185:4988-92.
26. Addison E G, North J, Bakhsh I, Marden C, Haq S, Al-Sarraj S, et al. Ligation of CD8alpha on human natural killer cells prevents activation-induced apoptosis and enhances cytolytic activity. Immunology 2005; 116:354-61.
27. Leonard W J, Lin J-X. Cytokine receptor signaling pathways. J Allergy Clin Immunol 2000; 105:877-88.
28. Li Q, Verma I M. NF-κB regulation in the immune system. Nat Rev Immunol 2002; 2:725-34.
29. André P, Denis C, Soulas C, Bourbon-Caillet C, Lopez J, Arnoux T, et al. Anti-NKG2A mAb is a checkpoint inhibitor that promotes anti-tumor immunity by unleashing both T and NK cells. Cell 2018; 175:1731-43.e13.
30. Srour E F, Leemhuis T, Jenski L, Redmond R, Jansen J. Cytolytic activity of human natural killer cell subpopulations isolated by four-color immunofluorescence flow cytometric cell sorting. Cytometry 1990; 11:442-6.
31. Ahmad F, Hong H S, Jackel M, Jablonka A, Lu I-N, Bhatnagar N, et al. High frequencies of polyfunctional CD8⁺ NK cells in chronic HIV-1 infection are associated with slower disease progression. J Virol 2014; 88:12397-408.
32. Geng J, Raghavan M. CD8αα homodimers function as a coreceptor for KIR3DL1. Proc Natl Acad Sci 2019; 116:17951-6.
33. Cheroutre H, Lambolez F. Doubting the TCR coreceptor function of CD8αα. Immunity 2008; 28:149-59.
34. Artyomov M N, Lis M, Devadas S, Davis M M, Chakraborty A K. CD4 and CD8 binding to MHC molecules primarily acts to enhance Lck delivery. Proc Natl Acad Sci 2010; 107:16916-21.
35. Cawthon A G, Lu H, Alexander-Miller M A. Peptide requirement for CTL activation reflects the sensitivity to CD3 engagement: correlation with CD8αβ versus CD8αα expression. J Immunol 2001; 167:2577-84.
36. Marin-Agudelo N M, Krasnick B, Becker-Hapak M, Berrien-Elliott M M, Foster M, Marsala L M, et al. Cytokine-induced memory-like NK cells exhibit enhanced autologous responses to metastatic melanoma. Soc Nat Immun 2019; abstract 69:101.
37. Marin N, Becker-Hapak M, Koch J, Berrien-Elliott M M, Foster M, Neal C, et al. Abstract 1546: The CD30/CD16A bispecific innate immune cell engager AFM13 elicits heterogeneous single-cell NK cell responses and effectively triggers memory-like (ML) NK cells. Immunology 2019; 1546.
38. Gang M, Mahn N D, Wong P, Neal C C, Marsala L, Foster M, et al. CAR-modified memory-like NK cells exhibit potent responses to NK-resistant lymphomas. Blood 2020 Jul. 2 [Epub ahead of print].
39. Foltz J A, Berrien-Elliott M M, Neal C, Foster M, McClain E, Schappe T, et al. Cytokine-induced memory-like (ML) NK cells persist for >2 months following adoptive transfer into leukemia patients with a MHC-compatible hematopoietic cell transplant (HCT). Blood 2019; 134:1954.
30. Bednarski J, Zimmerman C, Cashen A F, Desai S, Foster M, Schappe T, et al. Adoptively transferred donor-derived cytokine induced memory-like NK cells persist and induce remission in pediatric patient with relapsed acute myeloid leukemia after hematopoietic cell transplantation. Blood 2019; 134:A3307.
41. Rouillard A D, Gundersen G W, Fernandez N F, Wang Z, Monteiro C D, McDermott M G, et al. The harmonizome: a collection of processed datasets gathered to serve and mine knowledge about genes and proteins. Database 2016.
42. Wagner J A, Rosario M, Romee R, Berrien-Elliott M M, Schneider S E, Leong J W, et al. CD56bright NK cells exhibit potent antitumor responses following IL-15 priming. J Clin Invest 2017; 127:4042-58.
43. Romee R, Cooley S, Berrien-Elliott M M, Westervelt P, Verneris M R, Wagner J E, et al. First-in-human phase 1 clinical study of the IL-15 superagonist complex ALT-803 to treat relapse after transplantation. Blood 2018; 131:2515-27.
44. Kotecha N, Krutzik P O, Irish J M. Web-based analysis and publication of flow cytometry experiments. Curr Protoc Cytom 2010; Chapter 10:Unit10.17.
45. Diggins K E, Ferrell P B, Irish J M. Methods for discovery and characterization of cell subsets in high dimensional mass cytometry data. Methods 2015; 82:55-63.
46. Cooper M L, Choi J, Staser K, Ritchey J K, Devenport J M, Eckardt K, et al. An “off-the-shelf” fratricide-resistant CAR-T for the treatment of T cell hematologic malignancies. Leukemia 2018; 32:1970-83.
47. Clement K, Rees H, Canver M C, Gehrke J M, Farouni R, Hsu J Y, et al. CRISPResso2 provides accurate and rapid genome editing sequence analysis. Nat Biotechnol 2019; 37:224-6.

### Example 2: Properties of Donor NK Cells that Affect Clinical Outcomes and Anti-Tumor Responses

This example shows CD8 was expressed on memory-like NK cells following differentiation, the development of the assays to test for associations with NK cell responses in an early phase clinical trial, and experiments to demonstrate that CD8 loss-of-function resulted in enhanced anti-tumor response.

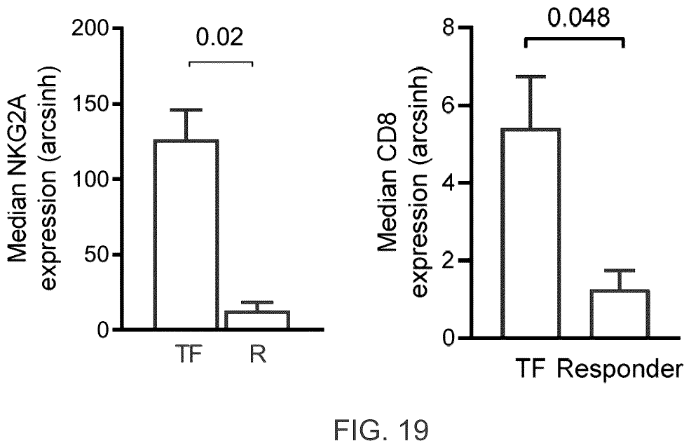

Median CD8 expression at D7 post transfer of donor NK cells into AML patients is associated with treatment failure in patients treated with ML NK cells (see e.g., FIG. 19). CD8 has not been previously identified as a negative predictor of response to NK cell therapy. CD8 has been associated with increased effector functions on conventional NK cells, making this observation nonobvious and unexpected. This observation will allow the prediction of response to NK cell therapy based on an interim short term assay. Here, mass cytometry assays were performed and data analyzed using CITRUS to test for associations with patient outcomes and NK cell phenotype.

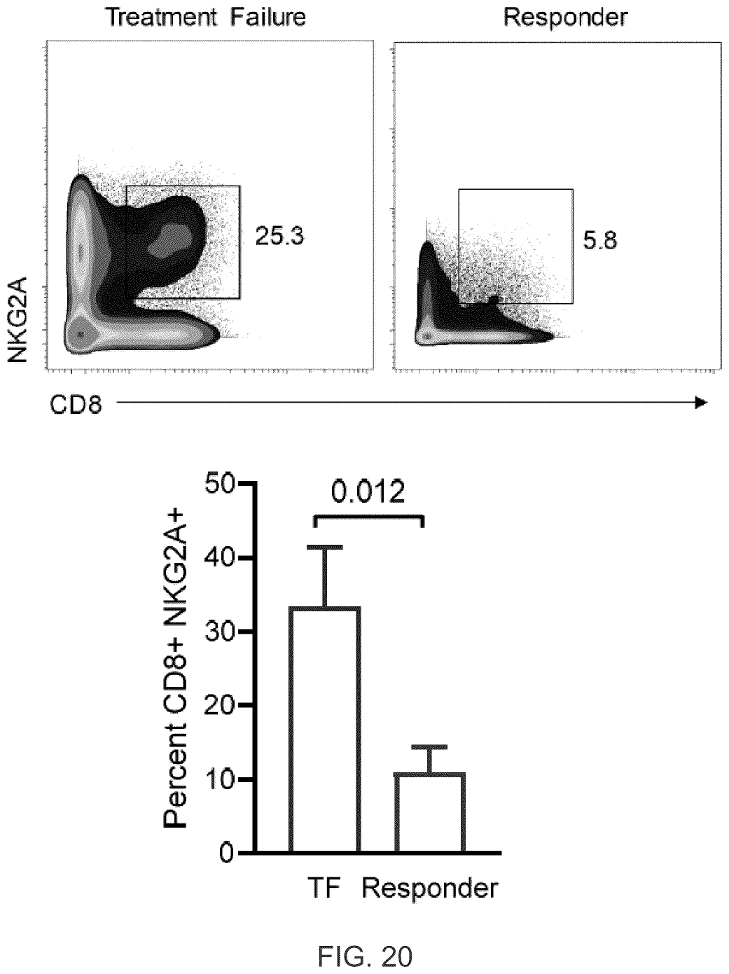

The frequency of double CD8⁺ NKG2A⁺ NK cells in the donor NK population at baseline (prior to manipulation or transfer) is associated with treatment failure (see e.g., FIG. 20). The combination of CD8 and NKG2A expression has never been reported as predicting response to NK cell therapy. Using this information, if multiple donors are available, a donor can be chosen with a favorable fraction of CD8⁺ NKG2A⁺ cells (e.g., a low or reduced fraction). This will also allow for the prediction of treatment response based on baseline NK cell attributes from a donor.

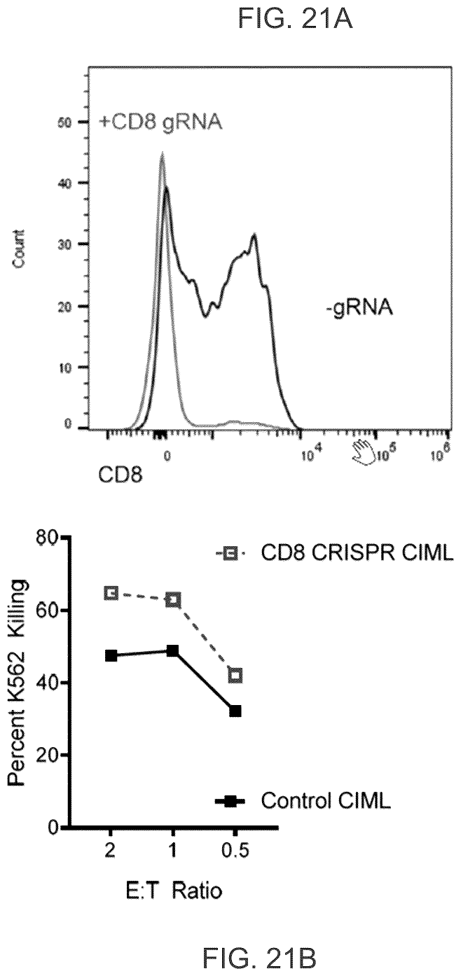

Removal or blockade of CD8 from NK cell populations enhances their anti-tumor effects (see e.g., FIG. 21A-FIG. 21B). CD8 has not previously been identified as a negative factor for (or inhibitor of) NK cell anti-tumor therapy response. CD8 has been associated with increased effector functions on conventional NK cells, making this observation nonobvious and unexpected. This discovery can be used in several ways to enhance NK cell anti-tumor responses. Examples include genetic modification of NK cells to remove (or reduce) CD8 expression, activity, or signaling, resulting in increased tumor cell killing, suggesting CD8 is inhibitory to NK cell anti-tumor response. Another example would be blockade of CD8 with monoclonal antibodies or other CD8 inhibiting agents.

CD8-negative cells expand more robustly to IL-12/15/18 than CD8⁺ NK cells. Results demonstrate that CD8⁺ NK cells have reduced proliferation in response to cytokine-induced memory-like differentiation than CD8-negative cells (see e.g., FIG. 22).

These discoveries help to solve the problem of weak NK cell anti-tumor responses to many tumor targets in serval ways. Using this information, it will also be possible to screen donors based on this criteria. Alternatively, strategies can be employed to inhibit CD8 (blockade/gene editing) on NK cells.

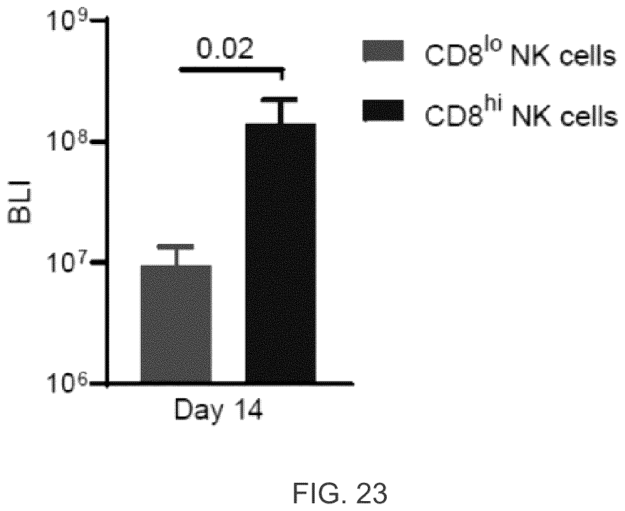

NSG mice were engrafted with 1e⁶ HLA-E⁺ K562-luciferase, then 5e⁶ IL-12/15/18 activated NK cells from healthy donors with hi (>50%) or lo (<10%) CD8⁺ NK cells were infused. NK cells were supported with 3 doses rhIL-15/week. Bioluminescent imaging was performed at D14 (FIG. 23). Data for days 4-29 post-infusion appears to show that CD8^{hi} control tumor early on and CD8^{lo} may perform better after proliferation initiates.

### Example 3: Generation of CIML NK Cells Clinical Trial Protocol

This example describes instructions and data capture for the production of Natural Killer Cells in overnight culture in cytokine containing medium. The NK cells are obtained by CD3 depletion and subsequent CD56 enrichment of an apheresis product.

TABLE 5
Reagents.

| Material Name | Manufacturer/Catalog # |
|---|---|
| CliniMACS PBS/EDTA Buffer | Miltenyi/700-25 |
| CliniMACS CD3 Reagent 7.5 mL | Miltenyi/273-01 |
| Human IVIG 1 gm/10 mL | Baxalta/00944-2700-02 |
| CliniMACS Depletion Tubing Set | Miltenyi/261-01 |
| CliniMACS CD56 Reagent 7.5 mL | Miltenyi/271-01 |
| CliniMACS Tubing Set | Miltenyi/161-01 |
| Human AB Sera | Sigma/H3667 |
| X-Vivo 15 | Lonza/04-744Q |
| IL-12 | Biolegend/573004 |
| IL-15 | Miltenyi/130-095-764 |
| IL-18 | InvivoGen/rhil-18 or |
| | Biovision/4179-1000 |
| 5% Buminate/HSA | Baxalta/0944-0495-05 |
| Hanks Balanced Salt Solution | Lonza/10-527F |
| HBSS) | |
| Human Serum Albumin (HSA) | Grifols/68516-5216-2 |

TABLE 6
Labware.

| | Manufacturer/ |
|---|---|
| Material Name | Catalog # |
| Blood transfusion filter | Haemonetics/SQ40 |
| 600 mL transfer pack | Fenwal/ |
| | 4R2023 |
| 300 mL transfer pack | Fenwal/ |
| | 4R2014 |
| 150 mL transfer pack | Fenwal/ |
| | 4R2001 |
| Blood administration set | Fenwal/4C2160 |
| Sterile 60 ml syringe | BD/309653 |
| Sterile 20 ml syringe | BD/302830 |
| Sterile 3 ml syringe | BD/309580 |
| Syringe needle | BD/305196 |
| Cryovials, 2.0 mL | Thermo Scientific/ |
| | 5000-0020 |
| 1 L filter unit | Corning/431098 or |
| | equivalent |
| 500 mL filter unit | VWR/97066-202 |
| Sterile 15 ml conical tube | Corning/430766 |
| Sterile 50 mL conical tube | Corning/430291 |
| Sterile 250 ml conical tube | Corning/430776 |
| Sterile 5 mL pipettes, | Greiner/606160 or |
| individually wrapped | equivalent |
| Sterile 10 mL pipettes, | Greiner/607160 or |
| individually wrapped | equivalent |
| Sterile 25 mL pipettes, | Greiner/760160 or |
| individually wrapped | equivalent |
| Sterile 50 mL pipettes, | Greiner/768160 or |
| individually wrapped | equivalent |
| Sterile alcohol pad | PDI/B60307 or |
| | equivalent |
| 20 ul pipette tips (barrier) | MidSci/AV20 or |
| | equivalent |
| 200 ul pipette tips (barrier) | MidSci/AV200 or |
| | equivalent |
| 1000 ul pipette tips (barrier) | MidSci/AV1250- |
| | H or equivalent |
| Cell culture bag | Saint Gobain/ |
| | VueLife |
| Sample site coupler | Fenwal/ |
| | 4C2405F |
| Transfer Set | Fenwal/4C2243 |
| Trypan Blue | Sigma/T8154 |
| Welding Wafers | Terumo/ |
| | SCW017 |
| Extension Sets | Baxter/2C6226 |
| 2 mL Aspirator Pipettes | Corning/357558 |

TABLE 7
Ancillary Equipment.

| | Manu- | Amount |
|---|---|---|
| Equipment | facturer | Required |
| Hemostats | Any | 3 |
| Tubing Roller | Any | 1 |
| Hemocytometer | Bright Line | 1 |
| Aspirator System | NA | 1 |
| Atmospheric Exposure Rack | NA | 1 |
| Crimper (if tube sealer unavailable) | Baxter | 1 |
| Crimps (if tube sealer unavailable) | Baxter | 1 box |

TABLE 8
Equipment.

| EQ # | Description | Serial Number |
|---|---|---|
| EQ279 | CliniMACS | 000621 |
| EQ280 | Sebra Tube Sealer | 2659 |
| EQ003 | Balance | 038QC6000 |
| EQ030 | Terumo Tubing Welder | 03100084 |
| EQ008 | BSC | 79192 |
| EQ132 | Centrifuge | ALA07D11 |
| EQ219 | 37° C. Incubator | 146798100711 |
| | Pipetaid | |
| | 20 μl pipetman | |
| | 200 μl pipetman | |
| | 1000 μl pipetman | |
| EQ282 | Rotator | NA |

### Clinical Manufacturing Process

Below are details regarding CD3 depletion, CD56 enrichment, activation, and formulation, and NKG2A and/or CD8 inhibition (e.g., deplete, reduce expression, knockout, large or small molecule inhibition or silencing, etc.).

1. (Optional): Select Donors for Low CD8, Low NKG2A, or Both.

Detect the amount of CD8+ and/or CD8−negative NK cells and/or detect the expression of NKG2A. If the CD8 expression and, optionally, NKG2A expression on the donor cells is a low fraction, the donor is considered a good candidate for donation.

2. Deplete Human Cells (Derived from Leukopheresis or Similar) of CD3+ Cells and Enrich CD56+NK Cells.

Prepare apheresis bag(s). Determine the volume of the Leukapheresis product. Mix the contents thoroughly by gently rotating the bag in hands. In the BSC, insert a sample site coupler into bag and withdraw 0.5 mL of Leukapheresis product using a 3 mL syringe. Transfer sample to a 2.0 mL Cryovial. Determine White Blood Cell count (WBC), CD3 percentage, and viability. After removing sample, insert a plasma transfer set into the Leukapheresis bag.

At any time, inject 20 mL of 25% Human Serum Albumin (HSA) into a 1 L CliniMACS PBS/EDTA Buffer bag (referred to as buffer throughout remaining BPR). Perform this step as needed throughout the procedure. Spike buffer bag through the grey septum with a plasma transfer set for steps involving buffer transfer. A buffer bag without a plasma transfer set is needed for CD3 depletion and CD56 selection on the CliniMACS.

Connect the Leukapheresis bag to a cell bag, split the starting product equally into cell bags. Bring each cell bag to approximately 600 mL of buffer. Weld a supernatant cell bag to the product cell bag.

Centrifuge the cells at: 200×g, 15 min, no brake, ambient temperature. Following centrifugation, break the weld seal and express supernatant from each bag. Calculate the total WBC count using the WBC described above and the Leukapheresis Product Volume. Calculate Total CD3 cells. Determine the number of CD3 Reagent vials needed when using CliniMACS Depletion 3.1.

All cells are to be in one cell bag for the addition of CD3 Reagent. Combine cells. Weigh cells after transferring all to one cell bag. Bring volume to 100 mL (±10%) with buffer if using one vial of CD3 Reagent, or to 200 mL (±10%) if using two vials. Heat seal off cell bag and ensure all cells are resuspended, leave enough tubing on cell bag to be able to weld to buffer bag after incubation with CD3 beads. Insert a sample site coupler into cell bag for addition of CD3 Reagent.

Reaction components to be injected in order stated. For 1 vial inject 1.5 mL IVIG Inject 3 mL IVIG. For 2 vials inject 1 vial CD3 Reagent Inject 2 vials CD3 Reagent. Disinfect the sample site coupler with an alcohol pad. Using a 3 mL syringe, inject IVIG into cell bag. Mix by rotating bag in hands, and allow at least a five minute incubation with IVIG before addition of CD3 Reagent. Using a 20 mL syringe, inject CD3 Reagent into cell bag. Start timer for 30 minutes when 1st vial of CD3 is injected. Note: Keep the IVIG in the BSC for use in the CD56 selection.

Inject air to the cell bag to allow for a convex shape. Mix the contents thoroughly by hand by using a gentle rotating motion. Place the pack flat on the rotator set to predetermined mark (approximately 25 rpm) for the remainder of the 30 minute incubation with the CD3 Reagent.

Calculate the total product WBC/mL based on resuspending the cells in 150 mL (1 vial scale) or 300 mL (2 vials scale) of buffer.

Turn on the CliniMACS at any time. The DEPLETION 3.1 program must only be used with CliniMACS® Depletion Tubing Set (Ref. 261-01).

The minimum and maximum WBC concentrations permissible are displayed in a box at the bottom left of the screen. If cell concentration is out of range, adjust the volume of buffer to obtain acceptable range. Enter WBC concentration and volume based on adjustment. The CliniMACS internal computer calculates the total number of labeled cells, the number of separation stages, the amount of buffer needed, and the liquid volumes to be collected in the Non-Target Cell bag, Buffer Waste Bag, and the Cell Collection Bag to verify that separation capacity is sufficient. After the first sample is complete, finish the selection using the remaining sample and a new tubing set. Combine cells after completing the second selection. Document new volumes and steps of second depletion.

When 30 minute product incubation is complete, bring volume to approximately 600 mL. Weld the cell bag to empty supernatant cell bag. Centrifuge the cells at: 300×g, 15 min, no brake, ambient temperature. Following centrifugation, express supernatant.

At any time, weld a 600 mL transfer bag to a standard blood administration set. Repeat with an additional 600 mL transfer bag and standard blood administration set.

Resuspend the cell pellet and bring volume to approximately 150 mL (or 300 mL if 2 vials of CD3 Reagent were used).

Spike cell bag with blood administration set and filter product. Repeat the filtration with 2^{nd} blood administration set so the product has been filtered twice to remove any possibility of micro clots. After 2^{nd} filtration, crimp seal or heat seal product bag.

Connect the Cell Preparation bag to the tubing set. Follow the prompts on the CliniMACS display. Note—Haemonetics filter is used upside down for this procedure. Verify that all bags of the tubing set are leveled correctly. Highest position is Buffer Bag. Middle Position is reapplication bag and non-target cell bag. Lowest position is cell Preparation bag.

At the beginning of the selection sequence, buffer is pumped upwards towards the Cell Preparation Bag to pre-fill the Haemonetics filter. Once the cells are loaded into the tubing set, the CliniMACS will display the remaining process time for the selection.

After selection is complete, record selection code.

Inside the BSC, insert a sample site coupler into Cell Collection Bag and withdraw 0.5 mL of product using a 3 mL syringe. Transfer sample to a 2.0 mL Cryovial. Determine WBC count and CD56 percentage. Estimate the number of cell bags (600 mL transfer packs) required. Note—Miltenyi recommends that the amount of buffer to product not exceed a 3:1 ratio.

Centrifuge the cells at: 300×g, 15 min, no brake, ambient temperature. Following centrifugation, break the weld seal and express off supernatant from each bag.

Calculate the total WBC count using the WBC results and the bag volume.

Calculate Total CD56 cells using results and the total WBC.

All cells are required to be in one cell bag for the addition of CD56 Reagent. Combine cells. Weigh cells after transferring to one cell bag (tare scale using empty 600 mL transfer pack). Crimp seal or heat seal cell bag and ensure all cells are resuspended. Leave enough tubing on cell bag to be able to weld to buffer bag after incubation with CD56 beads. Inside the BSC, insert sample site coupler into cell bag for addition of CD56 Reagent.

Reaction components are to be injected in order stated. Using a 3 mL syringe, inject 1.5 mL IVIG into cell bag. Using a 20 mL syringe, inject 1 vial of CD56 Reagent into cell bag. (Note: 5 minute pre-incubation with IVIG is not required before injecting CD56 Reagent for this step). After addition of CD56 Reagent, start timer for 30 minutes.

Inject air to the bag to allow for a convex shape. Mix the contents thoroughly by hand by using a gentle rotating motion. Place the pack flat on the rotator set to predetermined mark (approximately 25 rpm) for the remainder of the 30 minute incubation with CD56 Reagent.

Calculate the total WBC/mL of the product based on resuspending the cells.

At any time, weld a female Luer onto a transfer pack and OPEN THE SEAL. Attach the transfer bag to the CliniMACS tubing set.

CliniMACS procedure. Enter the WBC/mL in ×10⁶ format. Enter the CD56 percent. Enter volume. The minimum and maximum WBC concentrations permissible are displayed in a box at the bottom left of the screen. If cell concentration is out of range, adjust the volume of buffer to obtain acceptable range. Enter WBC concentration and volume based on adjustment.

The internal computer calculates the total number of labeled cells, the number of separation stages, and the amount of buffer needed.

When 30 minute product incubation with CD56 Reagent is complete, bring volume to approximately 600. Weld cell bag to empty supernatant cell bag. Centrifuge the cells at: 300×g, 15 min, no brake, ambient temperature. Following centrifugation, break the weld seal and express off supernatant from each bag.

At any time, weld a transfer bag to a standard blood administration set.

Resuspend the cell pellet and weld to buffer, bring volume to approximately 150 mL. Crimp seal or heat seal cell bag. Inside the BSC, spike cell bag with blood administration set and filter product. After filtration, crimp seal or heat seal cell bag. Connect the cell bag to the tubing set. Follow the prompts on the CliniMACS display. When the CliniMACS is ready to run the selection, the display will show “Selection/Ready for Enrichment 1.1/To Start Selection/Press RUN/Enrichment 1.1”. Once the cells are loaded into tubing set, the CliniMACS will display the remaining process time for the selection.

After selection is complete, record selection code. Transfer cells into conical tube. Centrifuge the tube containing the cells at: 660×g, Room Temperature, 10 min, high brake.

3. Activate and Culture NK Cells

At any time, the CIML NK culture media can be prepared. Filter an entire bottle of heat inactivated human AB serum (100 mL) thru a 0.22 um filter unit (PES500 mL). It is permissible to use multiple filters. Filtered serum may be stored between 2-8° C. for up to 30 days. Add the pre-filtered heat inactivated human AB serum and 1000 mL of X-Vivo 15 media to a 0.22 μm filter unit (PES 1000 mL). Filter the media. Cap the collection bottle with the lid provided and mix by inversion. Serum concentration in CIML NK culture media should be between 8-10%. CIML NK culture media can be stored at 4° C. for two weeks or at room temperature or at 37° C. for 24 hours.

Calculate the Patient CIML NK Dose and Goal Dose Activation Number. Patient CIML NK Target Dose: 0.5×10⁶/kg−1.0×10⁷/kg (max). Note—Use the maximum target dose of 1.0×10⁷/kg (max) for calculations.

Using cells set aside from above, perform a cell count.

Calculate average values by column.

When centrifugation of cells is complete, aspirate supernatant in the BSC and resuspend pellet at a final concentration of 2.0×10⁶ cells/mL in CIML NK Culture Media. Note: Final re-suspension concentration of 2.0×10⁶ cells/mL is ideal, but a concentration range of 1.0×10⁶ cells/mL-5.0×10⁶ cells/mL is acceptable. If a concentration other than 2.0×10⁶ cells/mL is used note it. Note: if re-suspension concentration is different than 2.0×10⁶ cells/mL use the true concentration for the above calculation: if re-suspension concentration is different than 2.0×10⁶ cells/mL use the true concentration for the above calculation.

Calculate number of cells to CIML activate. Note: if re-suspension concentration is different than 2.0×10⁶ cells/mL use the true concentration for the above calculation.

Add cytokines to the cells (each stock of cytokines is at a 1000× concentration, each vial of cytokines contains 100 ul). If more than one vial of cytokines is needed, pool cytokines into one vial before addition. After addition of cytokines, gently swirl contents. Final concentrations: between about 1 ng/mL and 100 ng/mL for each cytokine.

Activated cells are referred to as CIML NK cells throughout the remainder of BPR.

Transfer the CIML NK cells to a VueLife Cell Culture Bag. If needed, clamp the bag to maintain a volume height of approximately 1 cm. Use a 60 mL syringe attached to the Luer lock of the VueLife bag as a funnel. Pipette the cells from the tube into the syringe. (Note: transfer cells with care; do not generate aerosols.) Repeat until all cells are in the culture bag.

Incubate at 37° C., 5% CO₂ for 12 to 18 hours on the atmosphere exposure rack. (Note: 12 hours is preferred for cell viability).

At any time, inside the BSC prepare a 0.5% Buminate/HSA rinse solution. Pipette 56 mL of 5.0% Buminate/HSA solution into a 500 mL bottle of HBSS. Swirl to mix. Label the bottle “0.5% Buminate/HSA rinse solution”.

After 12-18 hour incubation, retrieve the CIML NK cells and CIML NK Culture media from the incubator. Examine the cells on the phase contrast microscope and then place them inside the BSC.

Decant CIML NK Cells into a centrifuge tube. Remove cells by gently pulling back the plunger. Transfer cells into a centrifuge tube.

Rinse the cell bag with CIML NK culture media. Gently massage the culture bag to remove any cells that may be stuck. Remove culture media. Transfer the rinse equally into tube(s) containing the cells.

Place the emptied & rinsed cell culture bag onto the phase contrast microscope and confirm that the majority of the cells have been recovered from the bag. If too many cells remain, perform an additional rinse of the bag. Record details.

Centrifuge the tube(s) containing the cells at: 660×g, Room Temperature, 10 min, high brake After centrifugation, move the tube(s) to the BSC.

Transfer 10 mL of supernatant to a 15 mL conical tube. Set aside for testing.

Resuspend pellet(s) with 200 mL of the 0.5% Buminate/HSA rinse solution. Pool the cells in one tube. Note: Initially resuspend pellet(s) in a small volume to break up the cells (recommended 10 mL or less). Centrifuge the cells at: 660×g, Room Temperature, 10 min, high brake Aspirate off remaining supernatant inside BSC. Resuspend pellet with 200 mL of the 0.5% Buminate/HSA rinse solution. Note: Initially resuspend pellet in a small volume to break up the cells (recommended 10 mL or less). Centrifuge the cells at: 660×g, Room Temperature, 10 min, high brake. Aspirate off remaining supernatant inside BSC. Resuspend pellet with 200 mL of the Buminate/HSA rinse solution previously prepared. Centrifuge the cells at: 660×g, Room Temperature, 10 min, high brake.

Transfer 10 mL of supernatant to a 15 mL conical tube for testing. Remove 1 mL of supernatant and place into a 2.0 mL Cryovial for endotoxin testing by BTCF staff. Remove 1 mL of supernatant and place into a 15 mL conical tube for Mycoplasma testing. Label tube with CIML UPN. Remove 1 mL of Rinse buffer and place into a separate 15 mL conical tube for control. Label tube with rinse. Aspirate off remaining supernatant inside BSC.

Assume a 50% loss of original activated cells (CIML NK cells). Calculate resuspension volume. Activated CIML NK cells×0.50=cells. Resuspend cells.

Transfer approximately 50 μl of cell suspension to a 2.0 mL Cryovial. Make a count dilution in another 2.0 mL Cryovial (recommended dilution range=20-40) and perform a cell count. Calculate average values by column. Adjust volume of CIML NK cells to a final cell concentration of 2.0×10⁶ cells/mL in 5% Buminate/HSA.

Calculate number of CIML NK Cells to provide to patient (dose 0.5×10⁶ cells/kg).

4. (Optional): Introduce Inhibitor of CD8 or NKG2A

At any step, the cells can be genetically modified or inhibited to reduce expression, activity, or signaling of CD8 or NKG2A as described herein.

1. A method of treatment of a cancer in a subject in need thereof, the method comprising;
a. administering to the subject an effective amount of a population of NK cells with reduced or no CD8 expression.

2. The method of claim 1, wherein the population of NK cells further exhibits reduced or no NKG2A expression.

3. The method of claim 2, wherein the population of NK cells comprises less that 20% of CD8+NKG2A+ cells.

4. The method of claim 1, wherein the NK cell population has a median NKG2A expression (measured in arcsinh) of less than 30 and a median CD8 expression (measured in arcsinh) of less than 2.5.

5. The method of claim 1, wherein the population of NK cells are engineered NK cells.

6. The method of claim 5, wherein the NK cells are produced by treating the NK cells with one or more of an anti-CD8 antibody or functional fragment or variant thereof, a short interfering RNA (siRNA) targeting CD8, an antisense oligonucleotide (ASO) targeting CD8; an inhibitory protein that antagonizes CD8; a protein expression blocker (PEBL) targeting CD8; or a fusion protein which is a decoy receptor for CD8, or a combination thereof.

7. The method of 5, wherein the engineered NK cells are obtained by a genetically modification process.

8. The method of claim 7, wherein the genetic modification process removes or reduces CD8 activity or expression by genome editing done using CRISPR-Cas nuclease system, TALENs, ZFNs, prime editors, or base editors.

9. The method of claim 6, wherein the inhibitory protein which antagonizes CD8 is selected from β-2 microglobulin and LPA5.

10. The method of claim 1, wherein the population of NK cells are isolated from a donor.

11. The method of claim 10, wherein the population of NK cells are treated with one or more cytokines, or one or more functional fragments or variants thereof, in an amount effective to expand NK cells into CD8-negative-enriched or CD8-depleted memory-like (ML) NK cells.

12. The method of claim 11, wherein the cytokines are selected from IL-12, IL-15, IL-18, or functional fragments or variants thereof, or any combination thereof.

13. The method of claim 1, wherein the cancer is AML, or a bone marrow tumor.

14. A method of treatment of a cancer in a subject in need thereof, the method comprising:
a. screening a donor for a population of NK cells for CD8 or NKG2A or a combination thereof;
b. isolating an effective population of NK cells comprising less than 20% of CD8⁺ NK cells or less than 20% NKG2A⁺ NK cells or a combination thereof; and
c. administering to the subject in need thereof the effective population of NK cells.

15. The method of claim 14, further comprising maintaining the isolated population of NK cells in the presence of cytokines, or one or more functional fragments or variants thereof prior to administration.

16. The method of claim 15, wherein the cytokines are selected from IL-12, IL-15, IL-18, or functional fragments or variants thereof, or any combination thereof.

17. The method of claim 15, wherein the NK cells are cytokine induced memory like (CIML) NK cells.

18. The method of claim 14, wherein the cancer is AML, or a bone marrow tumor.

19. A method of treatment of a cancer in a subject in need thereof, the method comprising; administering into the subject, an effective amount of an population of NK cells with reduced or no CD8 expression; anti-CD8 antibody or functional fragment or variant thereof, a short interfering RNA (siRNA) targeting CD8, an antisense oligonucleotide (ASO) targeting CD8; an inhibitory protein that antagonizes CD8; a protein expression blocker (PEBL) targeting CD8; or a fusion protein which is a decoy receptor for CD8, a CRISPR-Cas9 system targeting CD8, or a combination thereof.

20. The method of claim 19, wherein the cancer is AML, or a bone marrow tumor.
